(19) United States
(12) Patent Application Publication
(10) Pub. No.: US 2023/0105412 A1
(43) Pub. Date: Apr. 6, 2023

(54) 5-HT3 RECEPTOR MODULATOR, THE CRYSTALLINE FORM, METHODS OF MAKING, AND USE THEREOF

(71) Applicants: Jinkun HUANG, Thousand Oaks, CA (US); Dejian XIE, Glenmont, NY (US); CHENGDU SCIMOUNT PHARMATECH CO., LTD., Chengdu, Sichuan (CN)

(72) Inventors: David MANNING, Orchard Park, NY (US); Peter GUZZO, Niskayuna, NY (US)

(21) Appl. No.: 17/904,144

(22) PCT Filed: Mar. 16, 2021
(86) PCT No.: PCT/US2021/022595

§ 371 Date: Aug. 12, 2022

(51) Int. Cl. C07D 519/00; C07C 55/14; C07C 309/29; C07C 55/10; C07C 57/15; C07C 63/08; C07C 59/255; C07C 59/265; C07C 57/10; C07C 59/245; C07C 309/04; C07C 65/11; C07C 55/12; C07D 207/16

(52) U.S. Cl. CPC C07D 519/00; C07C 55/14; C07C 309/29; C07C 55/10; C07C 57/15; C07C 63/08; C07C 59/255; C07C 59/265; C07C 57/10; C07C 59/245; C07C 309/04; C07C 65/11; C07C 55/12; C07D 207/16; C07B 2200/13

(57) ABSTRACT

The present disclosure discloses free form or base and salts of compound of formula (I). Said salts include adipate, benzenesulphonate, hydrobromide, fumarate, benzoate, methanesulfonate, L-malate, d-glyconate, sorbate, phosphate, sulfate, L-tartrate, p-methylbenzenesulphonate, citrate, hydrochloride, ethanesulfonate, 1-hydroxy-2-naphthoate, succinate, acetate, glutarate or L-pyroglutamate. The present disclosure also discloses the crystals of free form and above salts.

### CROSS-REFERENCE TO RELATED APPLICATION

This application claims the priority of a U.S. Provisional application filed on Mar. 16, 2020 in the U.S. Patent and Trademark Office and assigned Ser. No. 62/990,228, which is hereby incorporated by reference in their entirety.

### TECHNICAL FIELD

The present disclosure relates to a serotonin type-3 (5-HT₃) receptor modulator, (S)-7-(quinuclidine-3-yl)-8,9-dihydro-2H-azepino[5,4,3-cd]indazol-6(7H)-one, its compositions, free form of, and the salts, and their use in the treatment of disease in which the 5-HT₃ receptor is implicated, for example, in the treatment of Irritable Bowel Syndrome (IBS), chemotherapy-induced nausea and vomiting (CINV), post-operative nausea and vomiting (PONV) and symptoms due to carcinoid syndrome. The present disclosure also relates to methods of preparation of the salts of 5-HT₃ receptor modulator.

### BACKGROUND

Irritable bowel syndrome (IBS) is a group of intestine functional disorders characterized by continual or interval attack and clinical situations of abdominal pain, abdominal distension, bowel evacuation habit and/or stool trait change, without gastrointestinal tract structure abnormality. IBS is classified as one of functional bowel disorders in Rome IV, and young and middle-aged people are the main patients. The age of onset usually is 20-50 years old, more women than men are diagnosed, and it has a tendency of familial aggregation. IBS is generally accompanied by other diseases of gastrointestinal tract functional disturbance such as functional dyspepsia.

Nausea and vomiting caused by chemotherapy remain among the most distressing side effects for patients undergoing treatment for cancer. Depending upon the chemotherapy agents or regimens given, up to 90% of patients may suffer from some form of chemotherapy-induced nausea and vomiting (CINV). Symptoms from CINV can be severely debilitating and often result in patients refusing further courses of chemotherapy, with obviously unfavorable consequences as regards to progression of the cancer. Furthermore, CINV is burdensome on the medical system, consuming time from the healthcare staff, who could otherwise attend to other patients or medical issues.

Postoperative nausea and vomiting (PONV) is the phenomenon of nausea, vomiting or retching experienced by a patient following a surgical procedure using anesthesia. It is an unpleasant complication that affects about 10% of the population undergoing general anesthesia each year.

Carcinoid syndrome is comprised of symptoms that occur secondary to carcinoid tumors. The syndrome includes diarrhea, flushing and vomiting and is associated with secretion of copious amounts of serotonin.

Studies indicate that 5-hydroxytryptamine (5-HT) is a key neurotransmitter in gastrointestinal tract, and the 5-hydroxytryptamine (5-HT₃) receptor is a key target point of developing drugs for treatment of irritable bowel syndrome, carcinoid syndrome, emesis, etc. The applicant's early studies show compounds with structure of formula (I) can effectively modulate the activity of the 5-HT₃ receptor, and have a very good application prospect in preparation of drugs for treatment of irritable bowel syndrome, carcinoid syndrome, emesis, etc.

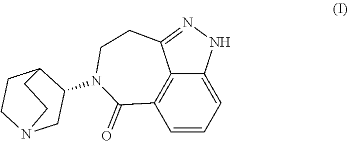 [SMILES: O=C1c2cccc3[nH]nc(c23)CCN1[C@@H]1CN2CCC1CC2]

The compound, (S)-7-(quinuclidin-3-yl)-8,9-dihydro-2H-azepino[5,4,3-cd]indazol-6(7H)-one or formula (I), may be useful in the treatment of inflammatory bowel disease (including but not limited to ulcerative colitis, pyoderma gangrenosum and Crohn's disease), irritable bowel syndrome, chemotherapy-induced nausea and vomiting, post-operative nausea and vomiting, carcinoid syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, drug/toxin-induced cognitive impairment (e.g., from alcohol, barbiturates, vitamin deficiencies, recreational drugs, lead, arsenic, mercury), disease-induced cognitive impairment (e.g., arising from Alzheimer's disease (senile dementia), vascular dementia, Parkinson's disease, multiple sclerosis, AIDS, encephalitis, trauma, renal and hepatic encephalopathy, hypothyroidism, Pick's disease, Korsakoff's syndrome and frontal and subcortical dementia), hypertension, bulimia, anorexia, obesity, cardiac arrhythmias, gastric acid hypersecretion, ulcers, pheochromocytoma, progressive supramuscular palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, attention deficit hyperactivity disorder (ADHD), Tourette's Syndrome, particularly, nicotine dependency, addiction and withdrawal; including use in smoking cessation therapy.

The compounds of this disclosure may also be used in a pharmaceutical composition in combination with an antidepressant such as, for example, a tricyclic antidepressant or a serotonin reuptake inhibiting antidepressant (SRI), in order to treat both the cognitive decline and depression associated with AD, PD, stroke, Huntington's chorea or traumatic brain injury (TBI); in combination with muscarinic agonists in order to stimulate both central muscarinic and nicotinic receptors for the treatment, for example, of ALS, cognitive dysfunction, age-related cognitive decline, AD, PD, stroke, Huntington's chorea and TBI; in combination with neurotrophic factors such as NGF in order to maximize cholinergic enhancement for the treatment, for example, of ALS, cognitive dysfunction, age-related cognitive decline, AD, PD stroke, Huntington's chorea and TBI; or in combination with agents that slow or arrest AD such as cognition enhancers, amyloid aggregation inhibitors, secretase inhibitors, tau kinase inhibitors, neuronal anti-inflammatory agents and estrogen-like therapy.

Compounds that relate to a serotonin type-3 (5-HT₃) receptor modulator, including (S)-7-(quinuclidin-3-yl)-8,9-dihydro-2H-azepino[5,4,3-cd]indazol-6(7H)-one or formula (I) are referred to in U.S. patent application Ser. No. 13/941,304, filed Jul. 12, 2013, now U.S. Pat. No. 8,710,047, which is a continuation of U.S. patent application Ser. No. 13/372,967, filed Feb. 14, 2012, now U.S. Pat. No. 8,501,729, which is a continuation of U.S. patent application Ser. No. 12/473,940, filed May 28, 2009, now U.S. Pat. No. 8,124,600, which claims benefit of U.S. Provisional Patent Application Ser. No. 61/057,014, filed May 29, 2008. The foregoing applications, owned in common with the present application and incorporated herein by reference in their entirety, generically recite pharmaceutically acceptable salts for the compounds referred to therein.

### SUMMARY OF THE EMBODIMENT

The present disclosure relates to the free form and salts of (S)-7-(quinuclidin-3-yl)-8,9-dihydro-2H-azepino[5,4,3-cd]indazol-6(7H)-one. The salts of the present disclosure comprise one or more of adipate, benzenesulphonate, hydrobromide, fumarate, benzoate, methanesulfonate, L-malate, d-glyconate, sorbate, phosphate, sulfate, L-tartrate, p-methylbenzenesulphonate, citrate, hydrochloride, ethanesulfonate, 1-hydroxy-2-naphthoate, succinate, acetate, glutarate or L-pyroglutamate salt.

In one embodiment, the present disclosure relates to the free form of (S)-7-(quinuclidin-3-yl)-8,9-dihydro-2H-azepino[5,4,3-cd]indazol-6(7H)-one. As set forth FIG. 7, in powder X-ray diffractometry, the free base has peak with no less than 10% relative intensity at diffraction angles (28) shown in the table A-1 below.

TABLE A-1
Diffraction angle 2θ (°)

11.3 ± 0.2
14.6 ± 0.2
17.2 ± 0.2
18.6 ± 0.2
21.8 ± 0.2
23.6 ± 0.2

In diffraction angle (28) values, errors sometimes occur, for example, attributable to the purity of crystal in powder applied to the powder X-ray diffraction analysis, particle size of the powder, an error derived from measure limit of the powder X-ray diffraction apparatus and the like. In this specification, when the crystal is specified by using diffraction angles (28), the diffraction angles are not limited to the measured values per se indicated as having peaks in the column of the examples. The diffraction angles are generally understood to have a variability of ±0.2° based upon recommendations outlined in the discussion of variability in the general chapter on X-ray powder diffraction in the United States Pharmacopeia, USP <941>. These are true to crystals, which will be described later, other than crystals of the free form, for example, methanesulfonate, phosphate, hydrochloride, succinate, 1-hydroxy-2-naphthoate and L-pyroglutamate salts.

As set forth FIG. 10, the free form of formula (I) is characterized in that it showed an endotherm at 242° C. by DSC indicative of melting.

In one embodiment, the present disclosure relates to methanesulfonate salt. As set forth FIG. 35 in powder X-ray diffractometry, the methanesulfonate salt had peaks with no less than 10% relative intensity at diffraction angles 26 shown in the table A-2 below.

| Diffraction angle | Relative intensity |
|---|---|
| 2θ (°) | (%) |
| 10.2 ± 0.2 | 57.7 |
| 11.3 ± 0.2 | 13.5 |
| 12.9 ± 0.2 | 19.5 |
| 17.0 ± 0.2 | 19.8 |
| 18.7 ± 0.2 | 14.5 |
| 19.6 ± 0.2 | 100.0 |
| 23.9 ± 0.2 | 25.3 |

As set forth FIG. 38, the methanesulfonate salt is characterized in that it showed a single endothermic event at 302° C. by DSC which was attributed to melting of the crystalline salt. The methanesulfonate salt is anhydrous in one embodiment.

In one embodiment, the present disclosure relates to phosphate salt. As set forth FIG. 48, in powder X-ray diffractometry, the phosphate salt has peaks with no less than 10% relative intensity at diffraction angles (28) shown in the table A-3 below.

TABLE A-3

| Diffraction angle | Relative intensity |
|---|---|
| 2θ (°) | (%) |
| 9.7 ± 0.2 | 15.8 |
| 12.3 ± 0.2 | 40.8 |
| 14.5 ± 0.2 | 16.3 |
| 15.1 ± 0.2 | 12.0 |
| 16.8 ± 0.2 | 33.6 |
| 19.5 ± 0.2 | 51.4 |
| 20.1 ± 0.2 | 100.0 |
| 21.3 ± 0.2 | 26.4 |
| 22.2 ± 0.2 | 23.5 |
| 25.2 ± 0.2 | 12.7 |
| 29.1 ± 0.2 | 12.9 |

As set forth FIG. 51, the phosphate salt is characterized in that it showed a single endothermic event at 276° C. by DSC which was attributed to melting of the crystalline salt. The phosphate salt is anhydrous in one embodiment.

In another embodiment, the present disclosure relates to hydrochloride salt. As set forth FIG. 76, in powder X-ray diffractometry, the hydrochloride salt has peaks with no less than 10% relative intensity at diffraction angles 26 shown in the table A-4 below.

TABLE A-4

| Diffraction angle | Relative intensity |
|---|---|
| 2θ (°) | (%) |
| 17.7 ± 0.2 | 100.0 |
| 21.5 ± 0.2 | 17.8 |
| 22.3 ± 0.2 | 43.9 |

As set forth FIG. 80, the hydrochloride salt is characterized in that it showed two endothermic events at 349° C. and 367° C. by DSC attributed to melting of the crystalline salt and decomposition. The hydrochloride salt is anhydrous in one embodiment.

In another embodiment, the present disclosure relates to the succinate salt. As set forth FIG. 91, in powder X-ray diffractometry, the succinate salt has peaks with no less than 10% relative intensity at diffraction angles (28) shown in the table A-5 below.

| Diffraction angle | Relative intensity |
|---|---|
| 2θ (°) | (%) |
| 6.5 ± 0.2 | 100.0 |
| 10.7 ± 0.2 | 25.7 |
| 14.2 ± 0.2 | 12.7 |
| 14.6 ± 0.2 | 23.6 |
| 17.4 ± 0.2 | 39.6 |
| 19.2 ± 0.2 | 64.2 |
| 20.2 ± 0.2 | 23.0 |
| 20.5 ± 0.2 | 40.8 |
| 21.4 ± 0.2 | 66.7 |
| 23.2 ± 0.2 | 28.9 |

As set forth FIG. 94, the succinate salt is characterized in that it showed a minor exotherm at the same temperature (273° C.) by DSC as the TGA step transition followed by melting at 220° C.

In another embodiment, the present disclosure relates to 1-hydroxy-2-naphthoate salt. As set forth FIG. 86, in powder X-ray diffractometry, the 1-hydroxy-2-naphthoate salt has peaks with no less than 10% relative intensity at diffraction angles (28) shown in the table A-6 below.

TABLE A-6

| Diffraction angle | Relative intensity |
|---|---|
| 2θ (°) | (%) |
| 6.5 ± 0.2 | 100 |
| 10.7 ± 0.2 | 26.5 |
| 14.2 ± 0.2 | 14.9 |
| 14.6 ± 0.2 | 24.4 |
| 17.4 ± 0.2 | 36.2 |
| 19.2 ± 0.2 | 55 |
| 20.2 ± 0.2 | 13.3 |
| 20.5 ± 0.2 | 39.8 |
| 21.4 ± 0.2 | 49 |
| 23.2 ± 0.2 | 20.4 |

As set forth FIGS. 89(a) and 89(b), the 1-hydroxy-2-naphthoate salt is characterized in that it showed a minor exotherm at the same temperature (200° C.) by DSC as the TGA step transition followed by melting at 220° C. The 1-hydroxy-2-naphthoate salt is anhydrous in one embodiment. In another embodiment, the 1-hydroxy-2-naphthoate salt is hydrate.

In another embodiment, the present disclosure relates to L-pyroglutamate salt. As set forth FIG. 100, in powder X-ray diffractometry, the L-pyroglutamate salt has peaks with no less than 10% relative intensity at diffraction angles (28) shown in the table A-7 below.

TABLE A-7

| Diffraction angle | Relative intensity |
|---|---|
| 2θ (°) | (%) |
| 5.5 ± 0.2 | 100.0 |
| 16.8 ± 0.2 | 17.9 |
| 17.5 ± 0.2 | 10.6 |
| 21.8 ± 0.2 | 13.2 |
| 22.4 ± 0.2 | 12.1 |

As set forth FIG. 103, the L-pyroglutamate salt is characterized in that it showed a single endothermic event at 241° C. which was attributed to melting of the crystalline salt. The L-pyroglutamate salt is anhydrous in one embodiment. In another embodiment, the L-pyroglutamate salt is a hydrate.

Another embodiment of the invention relates to a pharmaceutical composition comprising at least one of polymorphic Forms A, B, C, or other forms of certain salts of (S)-7-(quinuclidin-3-yl)-8,9-dihydro-2H-azepino[5,4,3-cd]indazol-6(7H)-one and a pharmaceutically acceptable carrier or excipient, for use in the treatment of inflammatory bowel disease (including but not limited to ulcerative colitis, pyoderma gangrenosum and Crohn's disease), irritable bowel syndrome, chemotherapy-induced nausea and vomiting, post-operative nausea and vomiting, carcinoid syndrome, spastic dystonia, chronic pain, acute pain, celiac sprue, pouchits, vasoconstriction, anxiety, panic disorder, depression, bipolar disorder, autism, sleep disorders, jet lag, amyotrophic lateral sclerosis (ALS), cognitive dysfunction, drug/toxin-induced cognitive impairment (e.g., from alcohol, barbiturates, vitamin deficiencies, recreational drugs, lead, arsenic, mercury), disease-induced cognitive impairment (e.g., arising from Alzheimer's disease (senile dementia), vascular dementia, Parkinson's disease, multiple sclerosis, AIDS, encephalitis, trauma, renal and hepatic encephalopathy, hypothyroidism, Pick's disease, Korsakoff's syndrome and frontal and subcortical dementia), hypertension, bulimia, anorexia, obesity, cardiac arrhythmias, gastric acid hypersecretion, ulcers, pheochromocytoma, progressive supramuscular palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, attention deficit hyperactivity disorder (ADHD), and Tourette's Syndrome. Another more preferred embodiment of the invention is wherein the pharmaceutical composition is useful in the treatment of nicotine dependency, addiction and withdrawal; most preferably, for use in smoking cessation therapy.

The present invention further relates to pharmaceutical compositions for the uses described in the foregoing paragraph comprising any one of the salts of (S)-7-(quinuclidin-3-yl)-8,9-dihydro-2H-azepino[5,4,3-cd]indazol-6(7H)-one.

The present invention further relates to a method of treating inflammatory bowel disease (including but not limited to ulcerative colitis, pyoderma gangrenosum and Crohn's disease), irritable bowel syndrome, chemotherapy-induced nausea and vomiting, post-operative nausea and vomiting, carcinoid syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, drug/toxin-induced cognitive impairment (e.g., from alcohol, barbiturates, vitamin deficiencies, recreational drugs, lead, arsenic, mercury), disease-induced cognitive impairment (e.g., arising from Alzheimer's disease (senile dementia), vascular dementia, Parkinson's disease, multiple sclerosis, AIDS, encephalitis, trauma, renal and hepatic encephalopathy, hypothyroidism, Pick's disease, Korsakoff's syndrome and frontal and subcortical dementia), hypertension, bulimia, anorexia, obesity, cardiac arrhythmias, gastric acid hypersecretion, ulcers, pheochromocytoma, progressive supramuscular palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, attention deficit hyperactivity disorder (ADHD), and Tourefte's Syndrome comprises administering to a subject in need of treatment a therapeutically effective amount of any of Forms A, B, C or other forms of the salt of formula (I) or (S)-7-(quinuclidin-3-yl)-8,9-dihydro-2H-azepino[5,4,3-cd]indazol-6(7H)-one.

The invention also relates to a process for the preparation of the Forms A, B or C of salts of (S)-7-(quinuclidin-3-yl)-8,9-dihydro-2H-azepino[5,4,3-cd]indazol-6(7H)-one comprising the steps of (i) contacting (S)-7-(quinuclidin-3-yl)-8,9-dihydro-2H-azepino[5,4,3-cd]indazol-6(7H)-one with between 1 and 2 equivalents of acid in a suitable solvent; and (ii) collecting the crystals formed.

A preferred embodiment of this disclosure relates to the above process wherein about 1.1 equivalents of acid is employed and the acid is added to a solution containing the free base. A preferred mode of practicing this process is wherein the contact step is allowed to proceed for less than 2 hours. A more preferred embodiment of this invention relates to the above process wherein the contact step (i.e., step “(i)” above) is allowed to proceed between 30-120° C.

Another preferred embodiment of this invention relates to the above process wherein the suitable solvent is selected from the group consisting of an (C₁-C₆)alkyl alcohol, an (C₁-C₆)alkyl ketone, an (C₁-C₆)alkyl ether, acetonitrile and an (C₁-C₆)alkyl ester (e.g., ethyl acetate, isopropyl acetate, etc.). More preferably, the suitable solvent is ethanol.

Further another embodiment relates to a polymorph of a compound having a structure represented by formula (I). Optionally, the polymorph is characterized substantially by at least one of the following powder x-ray diffraction pattern peak angles expressed in terms of 2θ (°) (±0.20°) as measured with copper Kα radiation chosen from 11.3, 14.6, 17.2, 18.57, 21.8 and 23.6.

Still another embodiment relates to a method for preparing a polymorph of a compound having a structure represented by formula (I). The method may comprise steps of subjecting to such compound to a temperature, such as room temperature, for example, in the presence of a medium selected from one or more of diisopropyl ether, ethanol, or isopropyl alcohol.

Optionally in any embodiment, the medium may comprise isopropyl alcohol.

Yet another embodiment relates to a pharmaceutical formulation. The pharmaceutical composition may comprise a polymorph of a compound having a structure represented by formula (I), and a pharmaceutically acceptable excipient.

Another embodiment relates to a method of treating a disease. The method may comprise steps of administering to a subject in need of such treatment a therapeutically effective amount of a polymorph of a compound having a structure represented by formula (I).

Still another embodiment relates to a polymorph of a compound having a structure represented by formula (I) for use as a medicament.

### BRIEF DESCRIPTION OF THE DRAWINGS

FIG. 1 is a DSC thermogram of Form A mono-glutamate salt from THF—lot HALG-26(40);

FIG. 2 is a moisture sorption curve of Form A mono-glutamate salt from THF—lot HAL-G-29(2);

FIG. 3 are Raman spectra: Form A glutamate salt—lot HAL-G-26(40), Form B glutamate salt—lot HAL-G-32(11), Form A, formula (I) free form—lot LMA-U-133(2). Unique Raman bands compared to free form I and within each respective salt form ⬇, red trace=free form;

FIG. 4 is a TGA thermogram of Form B mono-glutamate salt—lot HAL-G-32(11);

FIG. 5 is a DSC thermogram of Form B mono-glutamate salt—lot HAL-G-32(11);

FIG. 6 is an HPLC calibration curve of formula (I) for determination of water solubility;

FIG. 7 is a stack plot of XRPD patterns: (a) Form A free base-lot LMA-U-133(2); (b) Form A, following moisture sorption analysis-lot LMA-U-133(2) AD; (c) Form A, following overnight room temperature water slurry-lot HAL-G-31(9); (d) Form B, IPA solvate of formula (I) —lot HAL-G-32(7);

FIG. 8 is a Raman spectra of ALB1137391 free form: Form A—lot LMA-U-133(2), Form B, IPA solvate of formula (I)—lot HAL-G-32(7). I indicate unique Raman bands, red trace=free form;

FIG. 9 is a stack plot of ¹H-NMR spectra: (a) Form A free base—lot LMA-U-133(2), unknown impurity resonances observed*; (b) Form B, IPA solvate of formula (I)—lot HAL-G-32(7);

FIG. 10 is a DSC thermogram of Form A free base—lot LMA-U-133(2);

FIG. 11 is a moisture sorption curve of Form A free base—lot LMA-U-133(2);

FIG. 12 is an optical micrograph of Form A free base—lot LMA-U-133(2);

FIG. 13 is a TGA thermogram of Form B, mono-IPA solvate of formula (I)—lot HAL-G-32(7);

FIG. 14 is a DSC thermogram of Form B, mono-IPA solvate of formula (I)—lot HAL-G-32(7);

FIG. 15 is a stack plot of XRPD patterns: (a) Form A adipate salt from EtOH—lot HAL-G-26(1); (b) Form A, adipate salt from IPA—lot HAL-G-26(2); (c) formula (I) free form—lot LMA-U-133(2);

FIG. 16 is an optical micrograph of Form A adipate salt from IPA—lot HAL-G-26(2);

FIG. 17 is ¹H-NMR spectrum of Form A adipate salt from IPA—lot HAL-G-26(2);

FIG. 18 is a DSC thermogram of Form A adipate salt from IPA—lot HAL-G-26(2);

FIG. 19 is a stack plot of XRPD patterns: (a) Form A besylate salt from THF—lot HAL-G-26(3); (b) Form B is besylate salt from IPA—lot HAL-G-26(4); (c) formula (I) free form—lot LMA-U-133(2);

FIG. 20 is a stacked ¹H-NMR spectra: (a) Form A besylate salt from THF—lot HAL-G-26(3); (b) Form B besylate salt from IPA—lot HAL-G-26(4);

FIG. 21 is a DSC thermogram of Form A besylate salt from THF—lot HAL-G-26(3);

FIG. 22 is an optical micrograph of Form B besylate salt from IPA—lot HAL-G-26(4);

FIG. 23 is a DSC thermogram of Form B besylate salt from IPA—lot HAL-G-26(4);

FIG. 24 is a stack plot of XRPD patterns: (a) Form A hydrobromide salt from THF—lot HAL-G-26(5); (b) Form A, hydrobromide salt from IPA—lot HAL-G-26(6); (c) formula (I) free form—lot LMA-U-133(2);

FIG. 25 is an optical micrograph of Form A hydrobromide salt from IPA—lot HAL-G-26(6);

FIG. 26 is a DSC thermogram of Form A hydrobromide salt from IPA—lot HAL-G-26(6);

FIG. 27 is a stack plot of XRPD patterns: (a) Form A fumarate salt from THF—lot HAL-G-26(9); (b) Form A, fumarate salt from IPA—lot HAL-G-26(10); (c) formula (I) free form—lot LMA-U-133(2);

FIG. 28 is an optical micrograph of Form A fumarate salt from THF—lot HAL-G-26(9);

FIG. 29 is ¹H-NMR spectrum of Form A fumarate salt from THF—lot HAL-G-26(9);

FIG. 30 is a DSC thermogram of Form A fumarate salt from THF—lot HAL-G-26(9);

FIG. 31 is a stack plot of XRPD patterns: (a) Form A benzoate salt from THF—lot HAL-G-26(11); (b) Form A, benzoate salt from IPA—lot HAL-G-26(12); (c) formula (I) free form—lot LMA-U-133(2);

FIG. 32 is an optical micrograph of Form A benzoate salt from IPA—lot HAL-G-26(12);

FIG. 33 is an ¹H-NMR spectrum of Form A benzoate salt from IPA—lot HAL-G-26(12);

FIG. 34(a) is a TGA thermogram of Form A benzoate salt from IPA—lot HAL-G-26(12);

FIG. 34(b) is a DSC thermogram of Form A benzoate salt from IPA—lot HAL-G-26(12);

FIG. 35 is a stack plot of XRPD patterns: (a) Form A mesylate salt from THF—lot HAL-G-26(13); (b) Form A, mesylate salt from IPA—lot HAL-G-26(14); (c) Form A mesylate salt following gravimetric moisture sorption—lot HAL-G-26(13) AD; (d) Form A mesylate salt following 7 days of equilibration in THF—Lot HAL-G-32(1); (e) Form A mesylate salt following 7 days of exposure to 60° C.—lot HAL-G-30(2); (f) formula (I) free form—lot LMA-U-133(2);

FIG. 36 is an optical micrograph of Form A mesylate salt from THF—lot HAL-G-26(13);

FIG. 37 is an ¹H-NMR spectrum of Form A mesylate salt from THF—lot HAL-G-26(13);

FIG. 38 is a DSC thermogram of Form A mesylate salt from THF—lot HAL-G-26(13);

FIG. 39 is a moisture sorption curve of Form A mesylate salt from THF—lot HAL-G-26(13);

FIG. 40 is a stack plot of XRPD patterns: (a) Form A malate salt from THF—lot HAL-G-26(15); (b) Form A, malate salt from IPA—lot HAL-G-26(16); (c) formula (I) free form—lot LMA-U-133(2);

FIG. 41 is an optical micrograph of Form A malate salt from IPA—lot HAL-G-26(16);

FIG. 42 is an ¹H-NMR spectrum of Form A malate salt from IPA—lot HAL-G-26(16);

FIG. 43 is a TGA thermogram of Form A malate salt from IPA—lot HAL-G-26(16);

FIG. 44 is a DSC thermogram of Form A malate salt from IPA—lot HAL-G-26(16);

FIG. 45 is a stack plot of XRPD patterns: (a) Form A sorbate salt from IPA—lot HAL-G-26(20); (b) LB137391 free form—lot LMA-U-133(2);

FIG. 46 is an ¹H-NMR spectrum of Form A sorbate salt from IPA—lot HAL-G-26(20);

FIG. 47 is a DSC thermogram of Form A sorbate salt from IPA—lot HAL-G-26(20);

FIG. 48 is a stack plot of XRPD patterns: (a) Form A phosphate salt from THF—lot HAL-G-26(21); (b) Form A, phosphate salt from IPA—lot HAL-G-26(22); (c) Form A phosphate salt following gravimetric moisture sorption—lot HAL-G-26(22) AD; (d) Form A phosphate salt following 7 days of equilibration in IPA—Lot HAL-G-32(6); (e) Form A phosphate salt following 7 days of exposure to 60° C.—lot HAL-G-30(3); (f) formula (I) free form—lot LMA-U-133(2);

FIG. 49 is an optical micrograph of Form A phosphate salt from THF—lot HAL-G-26(21);

FIG. 50 is an ¹H-NMR spectrum of Form A phosphate salt from THF—lot HAL-G-26(21);

FIG. 51 is a DSC thermogram of Form A phosphate salt from IPA—lot HAL-G-26(22);

FIG. 52 is a moisture sorption curve of Form A phosphate salt from IPA—lot HAL-G-26(22);

FIG. 53 is a stack plot of XRPD patterns: (a) Form A sulfate salt from THF—lot HAL-G-26(23); (b) Form B sulfate salt from IPA—lot HAL-G-26(24); (c) formula (I) free form—lot LMA-U-133(2);

FIG. 54 is an optical micrograph of Form A sulfate salt from THF—lot HAL-G-26(23);

FIG. 55 is a stacked ¹H-NMR spectra: (a) Form A sulfate salt from THF—lot HAL-G-26(23); (b) Form B sulfate salt from IPA—lot HAL-G-26(24);

FIG. 56 is a TGA thermogram of Form A sulfate salt from THF—lot HAL-G-26(23);

FIG. 57 is a DSC thermogram of Form A sulfate salt from THF—lot HAL-G-26(23);

FIG. 58 is an optical micrograph of Form B sulfate salt from IPA—lot HAL-G-26(24);

FIG. 59 is a TGA thermogram of Form B sulfate salt from IPA—lot HAL-G-26(24);

FIG. 60 is a DSC thermogram of Form B sulfate salt from IPA—lot HAL-G-26(24);

FIG. 61 is a stack plot of XRPD patterns: (a) Form A tartrate salt from THF—lot HAL-G-26(25); (b) Form B tartrate salt from IPA—lot HAL-G-26(26); (c) formula (I) free form—lot LMA-U-133(2);

FIG. 62 are stacked ¹H-NMR spectra: (a) Form A tartrate salt from THF—lot HAL-G-26(25); (b) Form B tartrate salt from IPA—lot HAL-G-26(26), asterisks indicate unknown impurities;

FIG. 63 is a DSC thermogram of Form A tartrate salt from THF—lot HAL-G-26(25);

FIG. 64 is a TGA thermogram of Form A tartrate salt from THF—lot HAL-G-26(25);

FIG. 65 is a TGA thermogram of Form B tartrate salt from IPA—lot HAL-G-26(26);

FIG. 66 is a DSC thermogram of Form B tartrate salt from IPA—lot HAL-G-26(26);

FIG. 67 is a stack plot of XRPD patterns: (a) Form A tosylate salt from THF—lot HAL-G-26(27), additional reflection (marked by 1) at 6.5° 28; (b) Form A tosylate salt from IPA—lot HAL-G-26(28); (c) formula (I) free form —lot LMA-U-133(2);

FIG. 68 is an optical micrograph of Form A tosylate salt from THF—lot HAL-G-26(27);

FIG. 69 is an ¹H-NMR spectrum of Form A tosylate salt from THF—lot HAL-G-26(27);

FIG. 70 is a DSC thermogram of Form A tosylate salt from THF—lot HAL-G-26(27);

FIG. 71 is a stack plot of XRPD patterns: (a) Form A citrate salt from MeCOH—lot HAL-G-26(29); (b) Form A citrate salt from IPA—lot HAL-G-26(31); (c) Form B citrate salt from THF—lot HAL-G-26(30); (d) formula (I) free form—lot LMA-U-133(2);

FIG. 72 is a stacked ¹H-NMR spectrum: (a) Form A citrate salt from MeCOH—lot HAL-G-26(29); (b) Form B citrate salt from THF—lot HAL-G-26(30);

FIG. 73 is a TGA thermogram of Form A citrate salt from MeCOH—lot HAL-G-26(29);

FIG. 74 is a DSC thermogram of Form A citrate salt from MeCOH—lot HAL-G-26(29);

FIG. 75(a) is a TGA thermogram of Form B citrate salt from THF—lot HAL-G-26(30);

FIG. 75 is a DSC thermogram of Form B citrate salt from THF—lot HAL-G-26(30);

FIG. 76 is a stack plot of XRPD patterns: (a) Form A hydrochloride salt from EtOH—lot HAL-G-26(32); (b) Form A, hydrochloride salt from IPA—lot HAL-G-26(33); (c) Form A hydrochloride salt from THF—lot HAL-G-26(34); (d) Form A hydrochloride salt from EtOH at a 200 mg scale—lot HAL-G-29(3); (e) Form A hydrochloride salt following gravimetric moisture sorption analysis—lot HAL-G-29(3) AD; (f) formula (I) free form—lot LMA-U-133(2);

FIG. 77 are Raman spectra: Form A HCl salt—lot HAL-G-29(3), Form A, formula (I) free form—lot LMA-U-133(2). Peak positions marked “I” indicate unique Raman bands, red trace=free form;

FIG. 78 is an optical micrograph of Form A hydrochloride salt from EtOH—lot HAL-G-26(32);

FIG. 79 is a liquid ¹H-NMR spectrum of hydrochloride salt from EtOH—lot HAL-G-26(32);

FIG. 80 is a DSC thermogram of Form A hydrochloride salt from IPA—lot HAL-G-26(33);

FIG. 81 is a moisture sorption curve of Form A hydrochloride salt from IPA—lot HAL-G-29(3);

FIG. 82 is a stack plot of XRPD patterns: (a) Form A hydrochloride salt from EtOH at a 200 mg scale—lot HAL-G-29(3); (b) Form A hydrochloride salt following 7 days of equilibration in IPA—Lot HAL-G-32(2); (c) Form A hydrochloride salt following 7 days of exposure to 60° C.—lot HAL-G-30(1);

FIG. 83 is a stack plot of XRPD patterns: (a) Form A esylate salt from THF—lot HAL-G-26(35); (b) formula (I) free form—lot LMA-U-133(2);

FIG. 84 is an ¹H-NMR spectrum of Form A esylate salt from THF—lot HAL-G-26(35);

FIG. 85 is a DSC thermogram of Form A esylate salt from THF—lot HAL-G-26(35);

FIG. 86 is a stack plot of XRPD patterns: (a) Form A naphthoate salt from THF—lot HAL-G-26(36); (b) Form B naphthoate salt following gravimetric moisture sorption analysis—lot HAL-G-26(36) AD; (c) Form B following 7 days of room temperature equilibration in IPA—lot HAL-G-32(5); (d) Form B following 7 days of storage at 60° C. —lot HAL-G-30(5); (e) Form C following overnight room temperature equilibration in water—lot HAL-G-31(6); (f) formula (I) free form—lot LMA-U-133(2);

FIG. 87 displays stacked ¹H-NMR spectra: (a) Form A naphthoate salt from THF—lot HAL-G-26(36); (b) Form B naphthoate salt from 7 day IPA slurry—lot HAL-G-32(5); (c) Form C naphthoate salt from overnight water slurry—lot HAL-G-31(6);

FIG. 88 is stacked TGA thermograms: (a) Form A naphthoate salt from THF—lot HAL-G-26(36); (b) Form B naphthoate salt from 7 day IPA slurry—lot HAL-G-32(5); (c) Form C naphthoate salt from overnight water slurry—lot HAL-G-31(6);

FIG. 89 (a) is a DSC thermograms: (a) Form A naphthoate salt from THF—lot HAL-G-26(36);

FIG. 89(b) is a DSC thermogram: Form B naphthoate salt from 7 day IPA slurry—lot HAL-G-32(5); Form C naphthoate salt from overnight water slurry—lot HAL-G-31(6);

FIG. 90 is a moisture sorption curve of Form A naphthoate salt from THF—lot HAL-G-26(36);

FIG. 91 is a stack plot of XRPD patterns: (a) Form A succinate salt from THF—lot HAL-G-26(37); (b) Form A succinate salt from THF at 200 mg scale—lot HAL-G-29(1); (c) Form A following moisture sorption analysis—lot HAL-G-29(1) AD; (d) Form A after 7 days of equilibration in IPA—lot HAL-G-32(4); (e) Form A after 7 days of storage at 60° C. —lot HAL-G-30(6); (f) formula (I) free form—lot LMA-U-133(2);

FIG. 92 is an optical micrograph of Form A succinate salt from THF—lot HAL-G-26(37);

FIG. 93 is ¹H-NMR spectrum of Form A succinate salt from THF—lot HAL-G-26(37);

FIG. 94 is a DSC thermogram of Form A succinate salt from THF—lot HAL-G-26(37);

FIG. 95 is a moisture sorption curve of Form A succinate salt from THF—lot HAL-G-29(1);

FIG. 96 is a stack plot of XRPD patterns: (a) Form A glutarate salt from THF—lot HAL-G-26(39); (b) formula (I) free form—lot LMA-U-133(2);

FIG. 97 is an optical micrograph of Form A glutarate salt from THF—lot HAL-G-26(39);

FIG. 98 is ¹H-NMR spectrum of Form A glutarate salt from THF—lot HAL-G-26(39);

FIG. 99 is a DSC thermogram of Form A glutarate salt from THF—lot HAL-G-26(39);

FIG. 100 is a stack plot of XRPD patterns: (a) Form A glutamate salt from THF—lot HAL-G-26(40); (b) Form A glutamate salt from THF at 200 mg scale—lot HAL-G-29(2); (c) Form A after 7 days of equilibration in IPA—lot HAL-G-32(3); (d) Form A after 7 days of storage at 60° C.—lot HAL-G-30(4); (e) Form A following moisture sorption analysis—lot HAL-G-29(2) AD; (f) Form A after 5 hours of storage at 95% RH—lot HAL-G-32(11); (g) formula (I) free form—lot LMA-U-133(2);

FIG. 101 is an optical micrograph of Form A mono-glutamate salt from THF—lot HAL-G-26(40);

FIG. 102 shows stacked ¹H-NMR spectra: (a) Form A glutamate salt from THF—lot HAL-G-26(40); (b) Form B glutamate salt after 5 hours of storage of Form A at 95% RH—lot HAL-G-32(11).

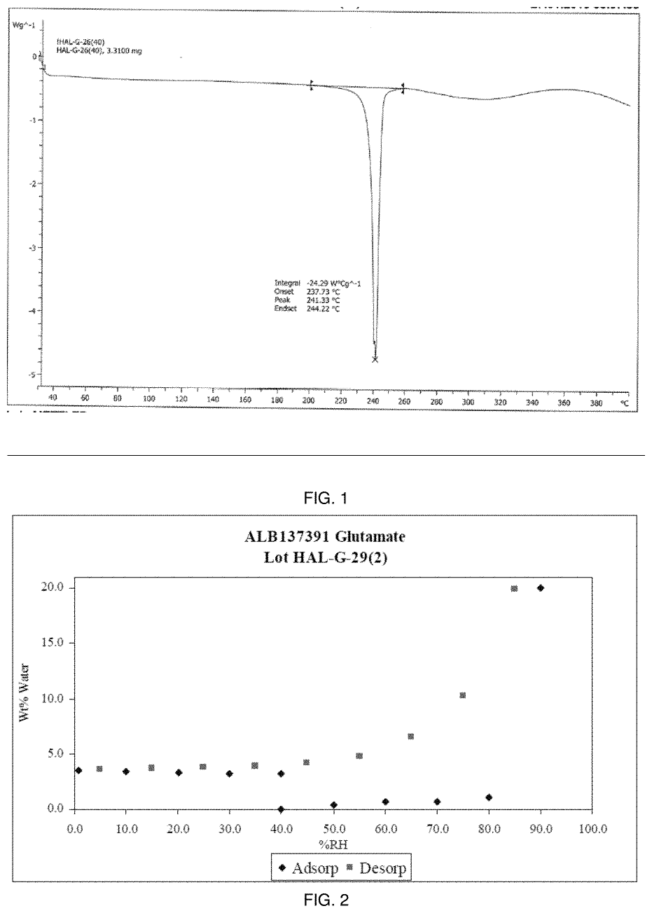

### DETAILED DESCRIPTION OF THE DISCLOSURE

The free form and salts of (S)-7-(quinuclidin-3-yl)-8,9-dihydro-2H-azepino[5,4,3-cd]indazol-6(7H)-one, such as adipate, benzenesulphonate, hydrobromide, fumarate, benzoate, methanesulfonate, L-malate, d-glyconate, sorbate, phosphate, sulfate, L-tartrate, p-methylbenzenesulphonate, citrate, hydrochloride, ethanesulfonate, 1-hydroxy-2-naphthoate, succinate, acetate, glutarate or L-pyroglutamate are relatively inert towards common excipients, making them highly suitable for pharmaceutical formulation use.

The object of the present disclosure is to provide free form and salts of relatively stable and soluble 5-HT₃ receptor modulator (formula (I)), as well as its crystal form, and the object of the present disclosure is also to provide the use of above salts or crystals in the preparation of drugs for prevention and/or treatment of 5-HT₃ receptor related diseases such as irritable bowel syndrome, carcinoid syndrome, emesis, etc.

The present disclosure provides the free form and salts of formula (I):

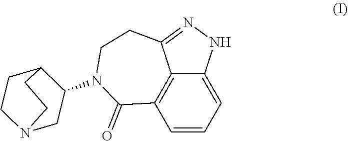 [SMILES: O=C1c2cccc3[nH]nc(c23)CCN1[C@@H]1CN2CCC1CC2]

As used above, and throughout the description of the disclosure, the following terms, unless otherwise indicated, shall be understood to have the following meanings.

The term “alkyl” means an aliphatic or cyclic hydrocarbon group which may be straight or branched having about 1 to about 6 carbon atoms in the chain. Branched means that one or more lower alkyl groups such as methyl, ethyl or propyl are attached to a linear alkyl chain. Example alkyl groups include methyl, ethyl, n-propyl, i-propyl, n-butyl, t-butyl, n-pentyl, and 3-pentyl.

The term “compound”, and equivalent expressions, are meant to embrace compounds of general formula (I) as hereinbefore described, which expression includes the prodrugs, the pharmaceutically acceptable salts, the oxides, and the solvates, e.g. hydrates, where the context so permits. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

The term “method of treating” means amelioration or relief from the symptoms and/or effects associated with the disorders described herein. As used herein, reference to “treatment” of a patient is intended to include prophylaxis.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. The present disclosure is meant to include all such possible isomers, as well as mixtures thereof, including racemic and optically pure forms. Optically active (R)- and (S)-, (−)- and (+)-, or (0)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

As used herein, and as would be understood by a person of skill in the art, the recitation of “a compound” is intended to include salts, solvates, oxides, and inclusion complexes of that compound as well as any stereoisomeric form, or a mixture of any such forms of that compound in any ratio. Thus, in accordance with some embodiments of the disclosure, a compound as described herein, including in the contexts of pharmaceutical compositions, methods of treatment, and compounds per se, is provided as the salt form.

The term “solvate” refers to a compound of formula (I) in the solid state, wherein molecules of a suitable solvent are incorporated in the crystal lattice. A suitable solvent for therapeutic administration is physiologically tolerable at the dosage administered.

Examples of suitable solvents for therapeutic administration are ethanol and water.

When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

Inclusion complexes are described in Remington, The Science and Practice of Pharmacy, 19th Ed. 1:176-177 (1995), which is hereby incorporated by reference in its entirety. The most commonly employed inclusion complexes are those with cyclodextrins, and all cyclodextrin complexes, natural and synthetic, are specifically encompassed within the claims.

The term “salt” refers to salts prepared from pharmaceutically acceptable non-toxic acids or bases including inorganic acids and bases and organic acids and bases. Since the compounds of formula (I) contain a basic nitrogen, salts may be prepared from pharmaceutically acceptable non-toxic acids including inorganic and organic acids.

Suitable pharmaceutically acceptable acid addition salts for the compounds of the present disclosure include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. When the compounds contain an acidic side chain, suitable pharmaceutically acceptable base addition salts for the compounds of the present disclosure include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), and procaine.

The configuration of any carbon-carbon double bond appearing herein is selected for convenience only and is not intended to designate a particular configuration; thus a carbon-carbon double bond depicted arbitrarily herein as E may be Z, E, or a mixture of the two in any proportion.

The term “therapeutically effective amount” is meant to describe an amount of compound of the present disclosure effective in modulating 5-HT₃ activity and thus producing the desired therapeutic effect. Such amounts generally vary according to a number of factors well within the purview of ordinarily skilled artisans given the description provided herein to determine and account for. These include, without limitation: the particular subject, as well as its age, weight, height, general physical condition, and medical history, the particular compound used, as well as the carrier in which it is formulated and the route of administration selected for it; and, the nature and severity of the condition being treated.

The term “pharmaceutical composition” means a composition comprising a compound of formula (I) and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms.

As used herein, the term “pharmaceutically acceptable carrier” is used to mean any carrier, diluent, adjuvant, excipient, or vehicle, as described herein. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances, Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates, Examples of lubricants include magnesium stearate, sodium lauryl sulphate, talc, as well as high molecular weight polyethylene glycols.

The term “pharmaceutically acceptable” means it is, within the scope of sound medical judgment, suitable for use in contact with the cells of humans and lower animals without undue toxicity, irritation, allergic response and the like, and are commensurate with a reasonable benefit/risk ratio.

One embodiment of the present disclosure relates to pharmaceutically acceptable salts, or non-salt forms, of any of the compounds of formula (d) described herein.

Single enantiomers, any mixture of enantiomers, including racemic mixtures, or diastereomers (both separated and as any mixtures) of the compounds of the present disclosure are also included within the scope of the disclosure.

The scope of the present disclosure also encompasses active metabolites of the present compounds.

Compounds of the present disclosure as described herein are useful as 5-HT₃ receptor modulators. It may be found upon examination that compounds that are not presently excluded from the claims are not patentable to the inventors in this application. In that case, the exclusion of species and genera in applicants' claims are to be considered artifacts of patent prosecution and not reflective of the inventors' concept or description of their disclosure. The disclosure, in a compound aspect, is all compounds of formula (I), except those that are in the public's possession.

While it may be possible for compounds of formula (I) to be administered as the raw chemical, it will often be preferable to present them as part of a pharmaceutical composition. Accordingly, another aspect of the present disclosure is a pharmaceutical composition containing a therapeutically effective amount of a compound of formula (I), or a pharmaceutically acceptable salt or solvate thereof, and a pharmaceutically acceptable carrier. The carrier must be “acceptable” in the sense of being compatible with the other ingredients of the formulation and not deleterious to the recipient thereof.

Furthermore, when reference is made in an independent claim to a compound or a pharmaceutically acceptable salt thereof, it will be understood that claims which depend from that independent claim which refer to such a compound also include pharmaceutically acceptable salts of the compound, even if explicit reference is not made to the salts.

In one embodiment of the present disclosure, the pharmaceutical composition further comprises one or more other therapeutic ingredients, e.g., other compounds effective in the treatment of IBS, CINV or PONV, that are known to persons of skill in the art. Such other therapeutic agents are described below.

Another aspect of the present disclosure relates to a method of treating a disease or condition which is susceptible to treatment with a 5-HT₃ receptor modulator. This method involves selecting a patient with a disease or condition which is susceptible to treatment with a 5-HT, receptor modulator and administering to the patient a therapeutically effective amount of a compound of formula (I) or a pharmaceutically acceptable salt thereof.

Diseases or conditions which are susceptible to treatment with a 5-HT, receptor modulator in accordance with the present disclosure include, but are not limited to, general anxiety disorders, social phobias, vertigo, obsessive-compulsive disorders, panic disorders, post-traumatic stress disorders, bulimia nervosa, drug withdrawal effects, alcohol dependency, pain (including visceral pain), sleep related central apneas, chronic fatigue syndrome, Parkinson's Disease Psychosis, schizophrenia, cognitive decline and defects in schizophrenia, Parkinson's Disease, Huntington's Chorea, presenile dementias, Alzheimer's Disease, psychological disorders, obesity, substance abuse disorders, dementia associated with neurodegenerative disease, cognition deficits, fibromyalgia syndrome (see US. Patent Application Publication No 2004/0204467, which is hereby incorporated by reference in its entirety), rosacea (see PCT Publication No. WO 2007/138233, which is hereby incorporated by reference in its entirety), cardiovascular disorders mediated by serotonin, chemotherapy induced nausea and vomiting (CINV), post-operative induced nausea and vomiting (PONV), radiation induced nausea and vomiting (RINV), gastrointestinal disorders (e.g. of the esophagus, stomach and both large and small intestines), including irritable bowel syndrome (IBS) and gastroesophageal reflux disease (GERD) (see European Patent No. EP0430190, U.S. Pat. Nos. 6,967,207, and 5,352,685, which are hereby incorporated by reference in their entirety), bronchial asthma, pruritus, migraine (see Costall et al., Current Drug Targets—CNS & Neurological Disorders, 3:27-37 (2004) and Israili, Current Med. Chem.—CNS Agents, 1:171-199 (2001), which are hereby incorporated by reference in their entirety), and epilepsy (see PCT Publication No. WO 2007/010275, which is hereby incorporated by reference in its entirety).

In another embodiment of the present disclosure, the above method further involves administering a therapeutically effective amount of one or more schizophrenia or Parkinson's Disease adjuncts. Suitable schizophrenia adjuncts include, but are not limited to, vaiproate and levomepromazine. Suitable Parkinson's Disease adjuncts include, but are not limited to, transdermal rotigatine, rotigatine and/or rasagiline as a levodopa adjunct, levodopa, carbidopa, doparnine agonists (brornocriptine, prarnipexole, or ropinirole), COMT inhibitors (entacapone or tolcapone), MAO-B inhibitors (rasagiline or selegiline), amantadine, anticholinergic agents (benztropine or trihexyphenidyl), and salfinamide. The compositions may additionally comprise aiprazolam, haloperidol, chlorpromazine, risperidone, paliperidone, olanzapine, ziprasidone, quetiapine, clozapine, lithium carbonate, diazepam, carbamazepine, selective serotonin re-uptake inhibitors (SSRI's) (ZOLOFT® or CELEXA®) or tricyclic antidepressants, such as PAMELOR®.

A further aspect of the present disclosure relates to a method of treating irritable bowel syndrome (IBS). This method involves selecting a patient with IBS and administering to the patient a therapeutically effective amount of a compound of formula (I) or a pharmaceutically acceptable salt thereof.

In another embodiment of the present disclosure, the above method further involves administering a therapeutically effective amount of other serotonin 5-HT₃ receptor modulators and/or serotonin 5-HT₄ receptor modulators, some of which are indicated below. Suitable other serotonin 5-HT; receptor modulators and/or serotonin 5-HT₄ receptor modulators include, but are not limited to, Alosetron (LOTRONEX®), renzapride, cilansetron, Tegaserod (ZELNORM®), Prucalopride, ondansetron; somatostatin analogs such as Octreotide; muscarinic receptor antagonists such as Darifenacin, and Zamifenacin; laxatives such as methylcellulose (CITRUCEL®), Psyllium (METAMUCIL®, FIBERALL®, REGULOID®, KONSYL®), malt soup extract, polyacrylic resins (e.g., hydrophilic forms such as polycarbophil and calcium polycarbophil), plantago seeds, dioctyl calcium sulfosuccinate, dioctyl potassium sulfosuccinate, dioctyl sodium sulfosuccinate, mineral oil, magnesium citrate, magnesium hydroxide, magnesium sulfate, dibasic sodium phosphate, monobasic sodium phosphate, sodium biphosphate, glycerin, anthraquinones or anthracene laxatives (such as aloe, cascara sagrada, danthron, senna, aloin, casanthranol, frangula, and rhubarb), diphenylmethanes (such as bisacodyl and phenolphthalein), and castor oil and the like; antispasmodics, such as the anticholinergic agents dicyclomine HCl (BENTYL®), hyoscyamine sulfate (LEVSIN®), and the like; antidepressants such as imipramine (TOFRANIL®), amitriptylin (ELAVIL®); antidiarrheal agents such as diphenoxylate HCl+atropine sulfate (LOMOTILD), loperamide (IMODILMO), natural or synthetic opiates (such as difenoxin, diphenoxylate, pargoric, opium tincture, and loperamide), anticholinergics (such as belladonna aikoloids-atropine hyoscyamine, and hyosine), acetyltannic acid, albumin tannate, alkofanone, aluminum salicylates, catechin, lidamidine, mebiquine, trillium, and uzarin, and the like; prokinetic agents, peripheral opiate narcotic antagonists such as fedotozine, trimebutine, and the like. Suitable prokinetic agents include, but are not limited to, cisapride monohydrate (PROPULSID®), metoclopromide, domperidone, and the like.

Another aspect of the present disclosure relates to a method of treating emesis. This method involves selecting a patient with emesis and administering to the patient a therapeutically effective amount of a compound of formula (I) or a pharmaceutically acceptable salt thereof.

In another embodiment of the present disclosure, the above method further involves administering a therapeutically effective amount of one or more other anti-emetic compounds. Suitable anti-emetic compounds include, but are not limited to, alosetron, aiprazolam, aprepitant, dexamethasone, dimenhydrinate, diphenhydramine, dolasetron, tetrahydrocannabinol, nabilone, dronabinol, droperidol, granisetron, haloperidol, lorazepam, metoclopramide, midazolam, olanzapine, ondansetron, palonosetron, proclorperazine, promethazine, and tropisetron.

Yet another aspect of the present disclosure relates to a method of treating CNS diseases or conditions. This method involves selecting a patient with a CNS disease or condition and administering to the patient an effective amount of a compound of formula (I) or a pharmaceutically acceptable salt thereof. Suitable CNS diseases or conditions include, but are not limited to, schizophrenia and Parkinson's disease. Beneficial effects of 5-HT, modulators have been reported in clinical studies of Parkinson's disease (Zoidan J et al., Advances in Neurology, 69:541-544 (1996), which is hereby incorporated by reference in its entirety) and schizophrenia (Zhang-Jin et al., Schizophrenia Research, 83: 102-110 (2006): Alder et al., Am, J, Psychiatry, 162:386-388 (2005), which are hereby incorporated by reference in their entirety). Brain responses in humans have been altered upon treatment with alosetron in IBS patients (Mayer et al., Aliment Pharmacol. Ther, 16:1357-1366 (2002), which is hereby incorporated by reference in its entirety). A 5-HT₃ modulator may be used as an adjunct or in combination with another medication.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

Another aspect of the present invention relates to a process of preparing a salt of formula (i). This process involves (i) contacting formula (i) or (S)-7-(quinuclidin-3-yl)-8,9˜dihydro-2H-azepino[5,4,3-cd]indazol-6(7H)-one in a suitable solvent with between about 1 and about 2 equivalents of acid; and (ii) collecting the crystals formed. Further another aspect of the present invention relates various salt polymorphs. The salt of formula (I) may exist as multiple polymorphs: anhydrous form, solvate form, or some other unsolvated form.

In some embodiments, one polymorph may convert to another polymorph under appropriate conditions.

Salts may be further purified by crystallization or recrystallization. The crystallization or recrystallization process may be standing or stirring. More specifically, the crystallization process may be preferably stirring. The temperature of the crystallization process may be −10˜60° C., preferably room temperature.

### Differential Scanning Calorimetry

The solid state thermal behavior of Forms A, B, C or other forms of the salt of (S)-7-(quinuclidin-3-yl)-8,9-dihydro-2H-azepino[5,4,3-cd]indazol-6(7H)-one were investigated by differential scanning calorimetry (DSC). The DSC thermograms were obtained on a Mettler Toledo DSC 822^{e}. Generally, samples between 1 and 10 mg were prepared in crimped aluminum pans with a small pinhole. The measurements were run at a heating rate of 10° C. per minute in the range of 30 to 400° C.

One of skill in the art will however note that in DSC measurements there is a certain degree of variability in actual measured onset and peak temperatures which is dependent on rate of heating, crystal shape and purity, and a number of measurement parameters.

### Powder X-Ray Diffraction Patterns

The powder x-ray diffraction patterns for Forms A, B, C or other forms of the salts were collected using a PANalytical CubiX-Pro XRD equipped with copper Kα radiation (CuKV, 45 kV), divergent slit (automatic 1.0 mm), and a Kevex solid state detector. Data was collected from 3.0 to 40.0 degrees in two theta (28) using a step size of 0.03 degrees and a step time of 10 seconds.

The x-ray powder diffraction pattern of the salt was conducted with a copper anode. The range for 26 was between 3.0 to 45.0 degrees with a step size of 0.03 degrees, a step time of 1.00 s, a smoothing width of 0.300° and a threshold of 1.0.

The diffraction peaks at diffraction angles (28) in a measured powder X-ray diffraction analysis for the Form A are shown in Table I. The relative intensities, however, may change depending on the crystal size and morphology.

The salts of the disclosure (hereafter “the active salts”) can be administered via either the oral, transdermal (e.g., through the use of a patch), intranasal, sublingual, rectal, parenteral or topical routes. Transdermal and oral administration are preferred. These salts are, most desirably, administered in dosages ranging from about 0.01 mg up to about 1500 mg per day, preferably from about 0.1 to about 300 mg per day in single or divided doses, although variations will necessarily occur depending upon the weight and condition of the subject being treated and the particular route of administration chosen. However, a dosage level that is in the range of about 0.001 mg to about 10 mg per kg of body weight per day is most desirably employed. Variations may nevertheless occur depending upon the weight and condition of the persons being treated and their individual responses to said medicament, as well as on the type of pharmaceutical formulation chosen and the time period and interval during which such administration is carried out. In some instances, dosage levels below the lower limit of the aforesaid range may be more than adequate, while in other cases still larger doses may be employed without causing any harmful side effects, provided that such larger doses are first divided into several small doses for administration throughout the day.

The active salts can be administered alone or in combination with pharmaceutically acceptable carriers or diluents by any of the several routes previously indicated. More particularly, the active salts can be administered in a wide variety of different dosage forms, e.g., they may be combined with various pharmaceutically acceptable inert carriers in the form of tablets, capsules, transdermal patches, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents. In addition, oral pharmaceutical compositions can be suitably sweetened and/or flavored. In general, the active compound is present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc can be used for tabletting purposes. Solid compositions of a similar type may also be employed as fillers in gelatin capsules; preferred materials in this connection also include lactose or milk sugar, as well as high molecular weight polyethylene glycols. When aqueous suspensions and/or elixirs are desired for oral administration the active ingredient may be combined with various sweetening or flavoring agents, coloring matter and, if so desired, emulsifying and/or suspending agents, together with such diluents as water, ethanol, propylene glycol, glycerin and various combinations thereof.

For parenteral administration, a solution of an active salt in either sesame or peanut oil or in aqueous propylene glycol can be employed. The aqueous solutions should be suitably buffered (preferably pH greater than 8), if necessary, and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

It is also possible to administer the active salts topically and this can be done by way of creams, a patch, jellies, gels, pastes, ointments and the like, in accordance with standard pharmaceutical practice.

The invention disclosure discloses that experiments were conducted through a series of salt formation investigation using multiple solvent and counterion combinations. The project proceeded in a multi-tiered approach to probe for crystalline salts of formula (I) including an initial screen at a 4 mg scale utilizing a diverse array of conditions and subsequent scale-up experiments. Formula (I) was confirmed to form crystalline and/or semi-crystalline salts with the following counterions: adipic acid, benzenesulfonic acid, hydrobromic acid, fumaric acid, benzoic acid, methanesulfonic acid, L-malic acid, sorbic acid, phosphoric acid, sulfuric acid, L-tartaric acid, p-toluenesulfonic acid, citric acid, hydrochloric acid, ethanesulfonic acid, 1-hydroxy-2-napthoic acid, succinic acid, glutaric acid and 2-pyrrolidine-5-carboxylic acid. Following the initial characterization, six salt candidates were identified for further assessment of physical properties including: methanesulfonic acid, phosphoric acid, hydrochloric acid, succinic acid, 1-hydroxy-2-napthoic acid and 2-pyrrolidine-5-carboxylic acid. The selections were made based on the following considerations: improved solubility over the free form, crystallinity, thermal stability (Mp), and safety. Each of the six salts was further characterized including an assessment of stability under slurry conditions and at elevated temperature/humidity. Based on the results from the analysis, a mono-HCl salt of formula (I) was recommended as the final salt form of formula (I). The hydrochloride exhibited the most desirable physical properties of the salts investigated including high solubility in water as well as form stability at elevated temperature, humidity and under slurry conditions. The phosphate and glutamate were also observed to be viable salts.

The phosphate was found to be slightly hygroscopic and showed improved aqueous solubility in comparison to the free form. The glutamate also showed good solubility, however, deliquesced at relative humidity (RH) conditions above 80% which potentially resulted in the formation of a stable monohydrate. The mesylate salt was not recommended due additional testing requirements that would be required for genotoxic impurities if isolated from an alcoholic solvent. The succinate and naphthoate were not recommended as these salts showed conversion to hydrate forms with limited RH stability. A summary of the experimental conditions used to generate all materials, evaluation at each stage of the salt screen and characterization of the salt species are provided herein.

Forty-one conditions that afforded solids which either showed birefringence, a unique Raman spectrum in comparison to the free form, or improved aqueous solubility were scaled up to 70 mg to provide additional material for confirmation of salt formation and further characterization. Crystalline or semi-crystalline solids recovered from these experiments were analyzed by XRPD, Raman, Optical Microscopy, DSC, TGA, ICP/OES, ¹H-NMR, and gravimetric solubility in water. Of the counterions investigated, crystalline and/or semi-crystalline salts of formula (I) were observed with adipic acid, benzenesulfonic acid, hydrobromic acid, fumaric acid, benzoic acid, methanesulfonic acid, L-malic acid, sorbic acid, phosphoric acid, sulfuric acid, L-tartaric acid, p-toluenesulfonic acid, citric acid, hydrochloric acid, ethanesulfonic acid, 1-hydroxy-2-napthoic acid, succinic acid, glutaric acid and L-pyroglutamic acid (2-pyrrolidine-5-carboxylic acid). Six salt forms were identified for further assessment of physical properties including: methanesulfonic acid, phosphoric acid, hydrochloric acid, succinic acid, 1-hydroxy-2-napthoic acid and 2-pyrrolidine-5-carboxylic acid (Table 7).

These salts were selected based on the following characteristics: improved water solubility compared to the free form, crystallinity, thermal stability as predicted by melting point(MP), and safety. Of these counterions, succinic acid, L-pyroglutamic acid and hydrochloric acid were scaled up to 200 mg to produce additional material for further characterization. The stability of each salt was assessed via room temperature slurries in organic solvent and water, storage at elevated temperature (60° C.), and exposure to elevated relative humidity by gravimetric moisture sorption. From the water slurries, the solubility of each salt was determined at ambient conditions. With the exception of the naphthoate, each of the six salts was observed to be stable following the slurry experiments and exposure to elevated temperature. The naphthoate was confirmed to be the least soluble salt exhibiting comparable water solubility to the free form. From the gravimetric moisture sorption experiments, the succinate, glutamate and naphthoate showed a propensity to form hydrates. Of these salts, only the glutamate exhibited a potential hydrate that was stable across a wide humidity range. The mesylate was observed to be moderately hygroscopic while the phosphate and hydrochloride showed the least affinity for water sorption. Overall there were many viable crystalline salt candidates identified; however, it was concluded that the hydrochloride possessed the most desirable physical properties including: stability, low hygroscopicity and substantially improved solubility over the formula (I) free form (Table 7).

Obviously, based on above content of the present disclosure, according to the common technical knowledge and the conventional means in the field, without department from above basic technical spirits, other various modifications, alternations or changes can further be made.

By following specific examples of said embodiments, above content of the present disclosure is further illustrated. But it should not be construed that the scope of above subject of the present disclosure is limited to following examples. The techniques realized based on above content of the present disclosure are all within the scope of the present disclosure.

### EXAMPLES

The following examples illustrate the methods and compounds of the present disclosure. It will be understood, however, that the disclosure is not limited to the specific Examples.

Unless otherwise noted, reagents and solvents were used as received from commercial suppliers. Formula (I) was synthesized according to US2009298809.

### Example 1 70 mg Scale Salt Formations

Albany Molecular Research, Inc. (AMRI) performed a salt screen of formula (I). The investigation was conducted through a series of salt-formation experiments using multiple solvent and counter ion combinations. The selected crystalline salt forms generated during the screen were analyzed by XRPD, the crystal form of each salt was classified into A, B, or C according to the XRPD results.

Approximately 70 mg of formula (I), lot LMA-U-133(2) was weighed to an 8-mL or 20-mL vial containing a magnetic stir bar. To the vial, primary solvent (MeOH, EtOH, IPA or THF) was added to ensure dissolution at elevated temperature. Following dissolution, 1.05 equivalent of acid was added drop-wise as a 0.25, or 0.50 M solution (Table 1). All mixtures were allowed to stir at elevated temperature for ˜10-15 min, followed by cooling to room temperature at a rate of 20° C./h and stirring at room temperature overnight. Solids observed following cooling were isolated by filtration and dried overnight under vacuum at room temperature. Samples without solids following cooling were evaporated under nitrogen and dried overnight under vacuum at room temperature. An analysis summary of the resulting solids is provided in Table 1.

TABLE 1
Results from 70 mg Scale Salt Formations

| | | | | ¹H-NMR | | | |
|---|---|---|---|---|---|---|---|
| | | | | (with) | DSC | | |
| | | XRPD | Acid:API | Residual | Peaks | TGA | |
| Counterion | Solvents | (Form) | Ratio | Solvent | ° C. | with loss | Comments |
| Adipic acid | IPA, | A | 0.5:1 | 0.4, IPA | 350 | No weight | — |
| | [EtOH] | | | | | loss | |
| Benzenesulfonic | THF | A | 1.0:1 | ND | 286, | No weight | — |
| acid | | | | | 181( [symbol] ) | loss | |
| Benzenesulfonic | IPA | B | 1.0:1 | 0.1, IPA | 188, | No weight | — |
| acid | | | | | 227( [symbol] ), [symbol] | loss | |
| Hydrobromic acid | IPA, [THF] | A | 1.0:1 | 0.5, IPA | 333 | No weight | — |
| | | | | | | loss | |
| L- [symbol] acid | IPA, [THF] | A | 0.5:1 | 0.4, IPA | — | — | — |
| | | | | (multiple | | | |
| | | | | impurities) | | | |
| Fumaric acid | THF, [IPA] | A | 1.0:1 | 1.6 (THF), | [symbol] | No weight | — |
| | | | | 0.4 (EtOH) | | loss | |
| Benzoic acid | IPA, [THF] | A | 1.0:1 | [symbol] , IPA | 351, 320 | No weight | — |
| | | | | | | loss | |
| Methanesulfonic | THF, [IPA] | A | 1.0:1 | 1.7, THF | 302 | No weight | — |
| acid | | | | | | loss | |
| L-Malic acid | IPA, [THF] | A | 0.4:1 | 0.4 (IPA), | 218, 278 | 2.0 | — |
| | | | | 1.1 (EtOH) | | | |
| D-glucoronic | THF, [IPA] | Salt not | — | Multiple | — | — | — |
| acid | | observed | | impurities | | | |
| Sorbic acid | IPA | A | [symbol] | 2.3, IPA | 135, 173, | [symbol] | Several |
| | | | | (multiple | 354 | | DSC [symbol] |
| | | | | impurities) | | | |
| Phosphoric acid | THF, [IPA] | A | [symbol] | ND | 276 | No | — |
| | | | | | | weight | |
| | | | | | | loss | |
| Sulfuric acid | THF | A | [symbol] | 5.4, EtOH | 174, 116 | 1.4 | Potential |
| | | | | | | | Hydrose |
| | | | | | | | or solvate |
| Sulfuric acid | IPA | B | [symbol] | 1.4 (IPA), | 315, 103 | 2.8 | Potential |
| | | | | 6.3 (EtOH) | | | Hydrose |
| | | | | | | | or solvate |
| L-tartaric acid | THF | A | [symbol] | [symbol] , EtOH | 72, 283 | [symbol] | — |
| L-tartaric acid | IPA | B | [symbol] | 2.3 (IPA), | 108, 284 | [symbol] | Dihydrose |
| | | | | 0.5 (EtOH) | | | |
| p-toluenesulfonic | THF | A | 1.0:1 | ND | 271 | No weight | — |
| acid | | | | | | loss | |
| p-toluenesulfonic | IPA | A | 1.0:1 | 0.2, IPA | — | No weight | — |
| acid | | | | | | loss | |
| Citric acid | MeOH, | A | 0.5:1 | 2.1, EtOH | [symbol] | 11.1 | Dihydrose |
| | [IPA] | | | | | | |
| Citric acid | THF | B | 0.5:1 | 1.1, EtOH | 237 | No weight | — |
| | | | | | | loss | |
| Hydrochloric | EtOH | A | 1.0:1 | 0.2, EtOH | 345, 176 | No weight | — |
| acid | [THF] | | | | | loss | |
| Hydrochloric | IPA | A | 1.0:1 | 0.2, IPA | 349, [symbol] | No weight | — |
| acid | | | | | | loss | |
| Ethanesulfonic | THF | A | 1.0:1 | 0.9, THF | [symbol] | No weight | — |
| acid | | | | | | loss | |
| 1-hydroxy-2- | THF | A | 1.1:1 | [symbol] THF | [symbol] | 2.9, 1.1 | — |
| napthoic acid | | | | | | | |
| Succinic acid | THF | A | 1.1:1 | 1.8 (THF), | 272 | No weight | — |
| | | | | 0.1 (EtOH) | | loss | |
| Acetic acid | THF | A | 0.5:1 | Multiple | — | — | — |
| | | | | impurities | | | |
| Glutaric acid | THF | A | [symbol] | 0.9 (THF), | 289 | No weight | — |
| | | | | 0.1 (EtOH) | | loss | |
| 2-pyrrolidine-5- | THF | A | 1.1:1 | ND | 241 | No weight | [symbol] |
| carboxylic acid | | | | | | loss | |
| L-proline | THF | Salt not | — | — | — | — | [symbol] |
| | | observed | | | | | |
| Free Form | diethylene, | A | — | ND | 242 | No weight | |
| | MeOH | | | | | loss | |

— not analyzed or applicable, [symbol]
[ ] additional solvent which afforded some form, data not collected [symbol]
indicates data missing or illegible when filed

### Example 2 200 mg Scale Salt Formations

To generate more material for further characterization, additional salt formations were performed for the HCl, glutamate and succinate salts of formula (I) at an increased scale. Approximately 200 mg of formula (I), lot LMA-U-133(2) was weighed to an 8 mL or 40 mL vial containing a magnetic stir bar. To the vial, primary solvent (EtOH or THE) was added to ensure dissolution at elevated temperature. Following dissolution, 1.05 equivalent of acid was added drop-wise as 0.5 M solution. All mixtures were allowed to stir at elevated temperature for ˜10-15 min, followed by cooling to room temperature at a rate of 20° C./h and stirring at room temperature overnight. Solids observed following cooling were isolated by filtration and dried overnight under vacuum at room temperature. See Table 2 for experimental details and results.

TABLE 2
Experimental Details and Results from 200 mg Scale Salt Formations

| | SM | | Solvent | | CI | | | | | |
|---|---|---|---|---|---|---|---|---|---|---|
| | Amt | | Amt | | Amt | Temp | | | Yield | XRPD |
| NB Code | (mg) | Solvent | (mL) | 1.05M eq. CI | (mL) | (° C.) | Precip | Isolation | (mg) | (Form) |
| HAL-G-29(3) | 200.8 | EtOH | 3.0 | HCl | 1.450 | 60 | Y | Filter | 193.5 | A |
| HAL-G-29(1) | 199.6 | THF | 17.0 | Succinic | 1.450 | 60 | Y | Filter | 224.5 | A |
| HAL-G-29(2) | 200.2 | THF | 17.0 | 2-pyrrolidine- | 1.450 | 60 | Y | Filter | 195.4 | A |
| | | | | 5-carboxylic | | | | | | |

### Example 3 Characterization of formula (I) Free Form and Salts

### 1. Methods

### (1) Characterization

The formula (I) free base starting material [lot LMA-U-133(2)] supplied by AMRI and select crystalline salt forms generated during the screen were analyzed by XRPD, Raman, Microscopy, DSC, and TGA. ¹H NMR and/or ICP/OES were performed to determine salt stoichiometry and to determine if degradation had occurred during the salt formation.

### (2) Gravimetric Solubility by TGA

The water solubility of select salts afforded from the 70 mg scale experiments was estimated using the following gravimetric method. Approximately 20 mg of each confirmed salt was weighed into a 2 mL HPLC vial. 50 μL increments of deionized H₂O were then added to each vial along with a small magnetic stir bar. If dissolution was observed, the samples were stored in a hood overnight and examined the next day for precipitation. Samples affording slurries after a maximum of 250 μl of water were left stirring at ambient conditions overnight to equilibrate. These samples were then centrifuge filtered, and 50 μL of each saturated solution was aliquoted into tared TGA pans. On the TGA, each sample was heated and held at elevated temperature until all solvent was dried off. Resulting dry weight and aliquoted volume for each saturated solution was then utilized in estimating solubility. Refer to Table 3 for results of this study.

TABLE 3
Gravimetric Water Solubility Determination

| | | | | | Volume | | | Estimated |
|---|---|---|---|---|---|---|---|---|
| | | | | Mass of | of DI | | Mass | Water |
| | Parent Lot | | XRPD | Sample | water | Dissolved | Recov. | Solubility |
| NB Code | Number | Counterion | (Form) | (mg) | (ml) | (Y/N) | (mg) | (mg/mL) |
| HAL-G-27(1) | HAL-G-26(2) | Adipic acid | A | 19.8 | 0.05 | Y | — | ≥400 |
| HAL-G-27(2) | HAL-G-26(3) | Benzenesulfonic | A | 19.4 | 0.05 | Y | — | ≥400 |
| | | acid | | | | | | |
| HAL-G-27(3) | HAL-G-26(4) | Benzenesulfonic | B | 20.4 | 0.05 | Y | — | ≥400 |
| | | acid | | | | | | |
| HAL-G-27(4) | HAL-G-26(6) | Hydrobromic acid | A | 19.7 | 0.25 | N | 2.803 | 56.1 |
| HAL-G-27(5) | HAL-G-26(9) | Fumaric acid | A | 20.6 | 0.05 | Y | — | ≥400 |
| HAL-G-27(6) | HAL-G-26(12) | Benzoic acid | A | 17.5 | 0.25 | N | 0.623 | 12.5 |
| HAL-G-27(7) | HAL-G-26(13) | Methanesulfonic | A | 21.4 | 0.05 | Y | — | ≥400 |
| | | acid | | | | | | |
| HAL-G-27(8) | HAL-G-26(16) | L-Malic acid | A | 19.5 | 0.25 | Y | — | ≥80 |
| HAL-G-27(9) | HAL-G-26(20) | Sorbic acid | A | 20.6 | 0.10 | Y | — | ≥200 |
| HAL-G-27(10) | HAL-G-26(21) | Phosphoric acid | A | 19.7 | 0.25 | N | 3.500 | 70.0 |
| HAL-G-27(11) | HAL-G-26(23) | Sulfuric acid | A | 19.0 | 0.25 | N | 0.946 | 18.9 |
| HAL-G-27(12) | HAL-G-26(24) | Sulfuric acid | B | 19.5 | 0.25 | N | 3.462 | 69.2 |
| LMA-U-133(2) | — | Free Form | A | — | — | N | — | <2.5 |
| HAL-G-27(13) | HAL-G-26(25) | L-tartaric acid | A | 18.7 | 0.20 | Y | — | ≥90 |
| HAL-G-27(14) | HAL-G-26(26) | L-tartaric acid | B | 19.6 | 0.15 | Y | — | ≥120 |
| HAL-G-27(15) | HAL-G-26(27) | p-toluenesulfonic | A | 18.5 | 0.35 | N | 1.014 | 20.3 |
| | | acid | | | | | | |
| HAL-G-27(16) | HAL-G-26(28) | p-toluenesulfonic | A | 18.8 | 0.35 | N | 0.869 | 17.4 |
| | | acid | | | | | | |
| HAL-G-27(17) | HAL-G-26(29) | Citric acid | A | 21.0 | 0.25 | N | 2.699 | 54.0 |
| HAL-G-27(18) | HAL-G-26(30) | Citric acid | B | 19.0 | 0.10 | Y* | — | ≥190 |
| HAL-G-27(19) | HAL-G-26(32) | Hydrochloric acid | A | 19.7 | 0.10 | Y | — | ≥190 |
| HAL-G-27(20) | HAL-G-26(33) | Hydrochloric acid | A | 19.2 | 0.10 | Y | — | ≥190 |
| HAL-G-27(21) | HAL-G-26(35) | Ethanesulfonic | A | 19.3 | 0.05 | Y | — | ≥280 |
| | | acid | | | | | | |
| HAL-G-27(22) | HAL-G-26(36) | 1-hydroxy-2- | A | 21.0 | 0.25 | N | 0.234 | 4.7 |
| | | napthoic acid | | | | | | |
| HAL-G-28(1) | HAL-G-26(37) | Succinic acid | A | 20.9 | 0.15 | Y | — | ≥149 |
| HAL-G-28(2) | HAL-G-26(39) | Glutaric acid | A | 21.6 | 0.05 | Y | — | ≥409 |
| HAL-G-28(3) | HAL-G-26(40) | 2-pyrrolidine-5- | A | 20.4 | 0.05 | Y | — | ≥400 |
| | | carboxylic acid | | | | | | |
| LMA-U-133(2) | — | Free Form | A | — | — | N | — | <2.5 |

* solid initially dissolved and ppt occurred after 2 hours of [symbol] at RT
— not analyzed or applicable [symbol]
indicates data missing or illegible when filed

### (3) Aqueous Solubility Study

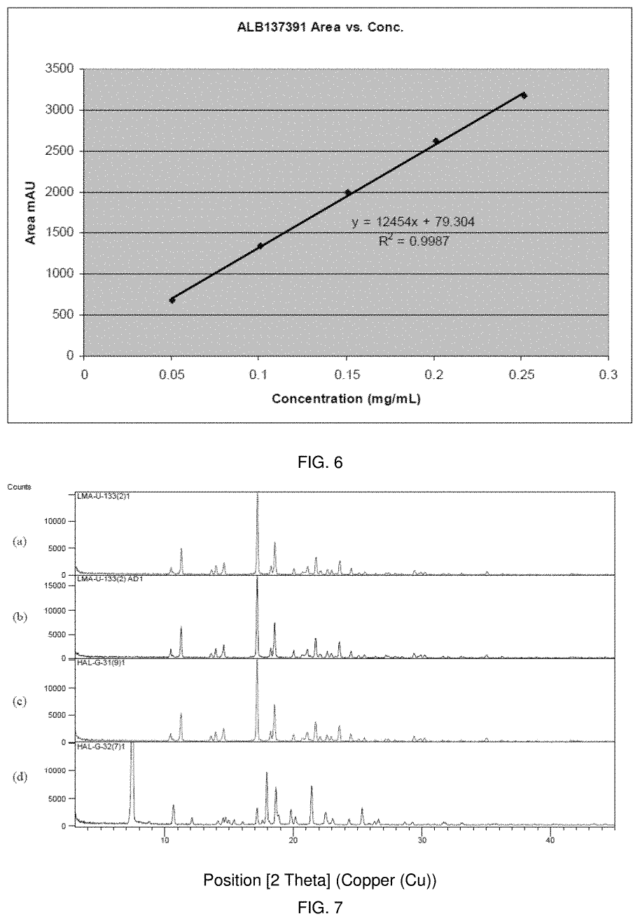

Approximately 20 mg of the free form, mesylate, hydrochloride, succinate, glutamate, naphthoate and phosphate salts of formula (I) were weighed to individual HPLC vials equipped with a magnetic stir bar and 0.05 mL increments of deionized water added. If dissolution was observed, the approximate solubility was recorded. If no dissolution had occurred, the slurries were stirred overnight at ambient temperature. The supernatant from each slurry was obtained using centrifuge filters. Each solution was tested for pH and by HPLG versus a calibration curve (FIG. 6) to determine the equilibrium solubility. Solids obtained from the filtration were analyzed by XRPD to confirm the crystalline form (Table 4).

TABLE 4
Water Solubility of formula (I) Salts and Free Form

| | | | | | Volume | | | | |
|---|---|---|---|---|---|---|---|---|---|
| | | | XRPD | Sample | of DI | | Aqueous | | |
| | | | Form | weight | Water | Dissolution | Solubility | | XRPD |
| Counterion | NB Code | Parent Lot # | (Initial) | (mg) | (mL) | (Y/N) | (mg/mL) | pH | (Form) |
| HCl | HAL-G-31(3) | HAL-G-26(34) | A | 20.9 | 0.10 | Y | ≥209 | — | — |
| Succinic | HAL-G-31(4) | HAL-G-29(1) | | 21.0 | 0.15 | Y | ≥140 | — | — |
| L-pyroglutamic | HAL-G-31(5) | HAL-G-29(2) | | 22.8 | 0.05 | Y | ≥400 | — | — |
| 1-hydroxy- | HAL-G-31(6) | HAL-G-29(36) | | 19.8 | 0.40 | N | 2.3 | 6.2 | C |
| 2-napthoic | | | | | | | | | |
| MSA | HAL-G-31(7) | HAL-G-29(14) | | 20.0 | 0.05 | Y | ≥400 | — | — |
| Phosphoric | HAL-G-31(8) | HAL-G-29(22) | | 19.9 | 0.25 | N | 51.9 | 3.5 | * |
| Free Form | HAL-G-31(9) | LMA-U-133(2) | | 21.6 | 0.23 | N | 2.0 | 9.3 | A |

* insufficient recovery of solids from slurry
— not analyzed or applicable

### (4) Stability at Elevated Temperature

Approximately 10 mg of the free form, mesylate, hydrochloride, succinate, glutamate, naphthoate and phosphate salts of formula (I) were weighed to individual 4 mL vials and stored uncovered in an oven at 60° C. and ambient pressure. After seven days of exposure, the solids were analyzed by XRPD to check for form conversion and ¹H-NMR for signs of degradation (Table 5).

TABLE 5
Elevated Temperature Stability of formula (I) Salt forms

| | | | Initial | | XRPD | |
|---|---|---|---|---|---|---|
| NB Code | Parent Lot # | Counterion | Form | Conditions | (Form) | ¹H-NMR |
| HAL-G-30(1) | HAL-G-29(3) | HCl | A | 60° C. | A | No |
| HAL-G-30(2) | HAL-G-29(13) | MSA | | (7 days) | A | degradation |
| HAL-G-30(3) | HAL-G-26(22) | Phosphoric | | | A | |
| HAL-G-30(4) | HAL-G-29(2) | L-pyroglutamic | | | A | |
| HAL-G-30(5) | HAL-G-26(36) | 1-hydroxy-2-napthoic | | | B* | |
| HAL-G-30(6) | HAL-G-29(1) | Succinic | | | A | |
| HAL-G-30(7) | LMA-U-133(2) | Free Form | | | A | |

*Similar pattern to Form B with additional reflections as 13, 13.5 and 14.5 degrees 2-theta

### (5) Organic Slurry Experiments

Approximately 30 mg of the free form, mesylate, hydrochloride, succinate, glutamate, naphthoate and phosphate salts of formula (I) were weighed to individual HPLG vials equipped with a magnetic stir bar. Either THF or IPA was added to obtain a free flowing slurry. After seven days of ambient equilibration, solid from each slurry was obtained using centrifuge filtration with 0.45 μm nylon centrifuge filters, and dried at ambient temperature and ˜30 in Hg. After drying overnight, each was analyzed by XRPD to check for form conversion and ¹H-NMR for signs of degradation (Table 6).

TABLE 6
Organic Slurries of formula (I) Salts

| | | | Sample | | | | |
|---|---|---|---|---|---|---|---|
| | | Parent | weight | | Volume | XRPD (Form) | |
| NB Code | Counterion | Lot (mg) | (mg) | Solvent | (mL) | Initial | 7 Days |
| HAL-G-32(1) | MSA | HAL-G-26(13) | 15.5 | THF | 0.25 | A | A |
| | | HAL-G-26(14) | 14.0 | IPA | | | |
| HAL-G-32(2) | HCl | HAL-G-26(33) | 7.3 | | 0.60 | | A |
| | | HAL-G-29(3) | 23.3 | | | | |
| HAL-G-32(3) | L-pyroglutamic | HAL-G-29(2) | 30.5 | | 0.35 | | A |
| HAL-G-32(4) | Succinic | HAL-G-29(1) | 30.3 | | 0.40 | | A |
| HAL-G-32(5) | 1-hydroxy-2-napthoic | HAL-G-26(36) | 27.9 | | 0.30 | | B |
| HAL-G-32(6) | Phosphoric | HAL-G-26(22) | 24.0 | | 0.30 | | A |
| HAL-G-32(7) | Free Form | LMA-U-133(2) | 27.7 | | 0.30 | | B |

### (6) Relative Humidity Study

Approximately 30 mg of Form A mono-glutamate salt [lot HAL-G-29(2)] was stored uncovered in a small glass container in a desiccator containing an aqueous saturated solution of Na₂HPO₄.12H₂O to achieve a relative humidity of 95%. After five hours of exposure the solid had deliquesced and the sample was exposed to the lab environment (20% RH) overnight. The resulting dried solid was characterized by XRPD, DSC, and TGA.

### 2. Results

### (1) Formula (I) Free Form

### Form A

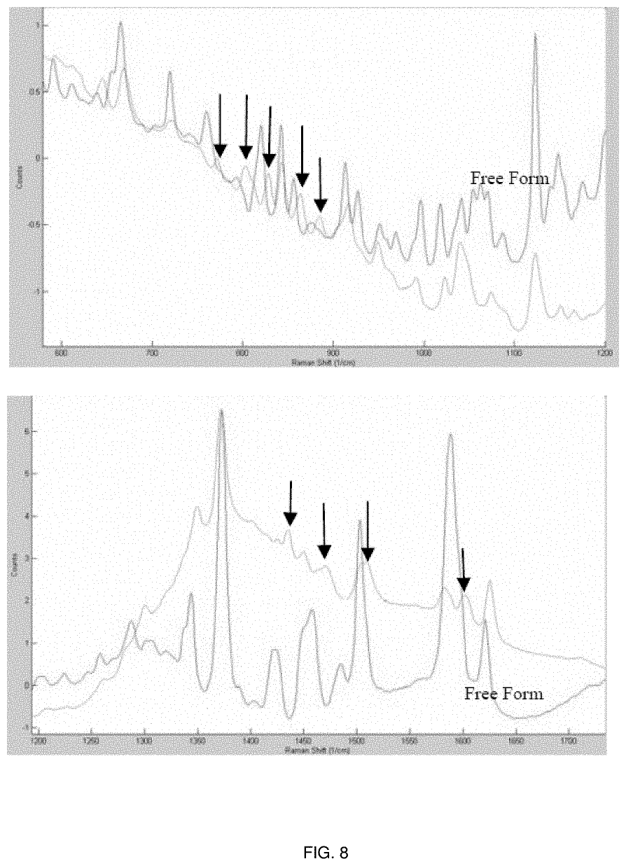

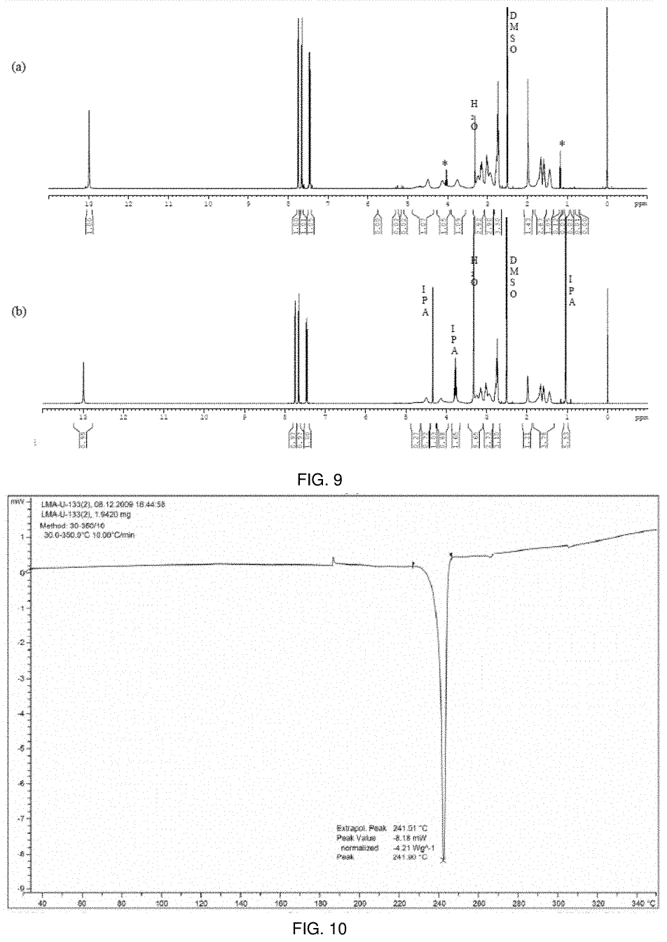

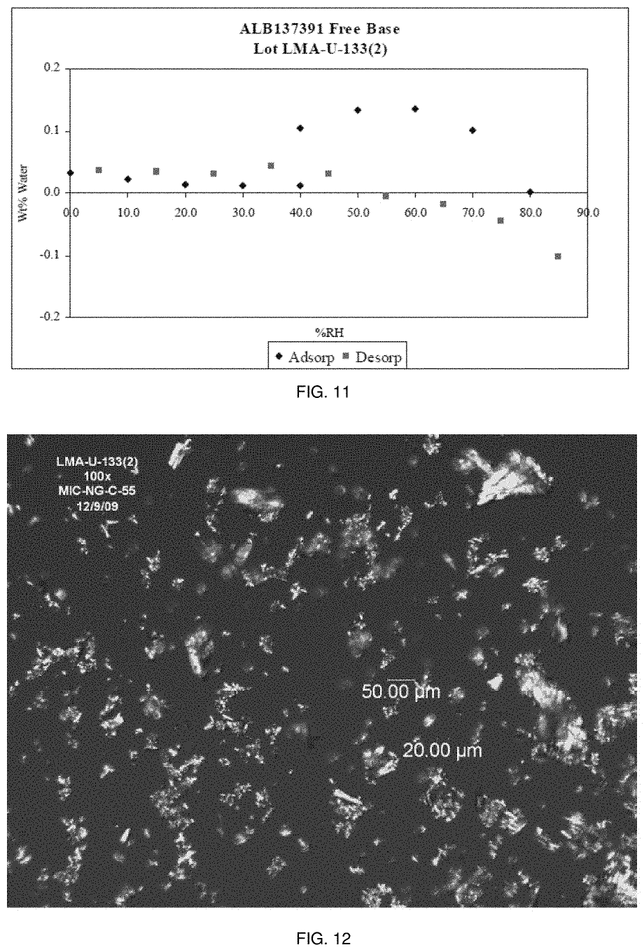

The formula (I) free form [Lot LMA-U-133(2)] obtained from the Medicinal Chemistry Department at AMRI, afforded a crystalline diffraction pattern by XRPD and was designated as Form A (FIG. 7). This crystalline form was also observed to be Raman active as shown in FIG. 8. ¹H-NMR analysis confirmed the chemical structure of formula (I) and showed two unknown impurity resonances at 1.2 and 4.0 ppm (FIG. 9). Thermal analysis by DSC showed an endotherm at 242° C. indicative of melting (FIG. 10). TGA analysis showed no weight loss. Moisture sorption analysis showed the material was non hygroscopic, adsorbing less than 0.2 wt % water throughout the humidity program (FIG. 11). XRPD analysis of the dried material following the moisture sorption program afforded an XRPD pattern that was consistent with the starting material, suggesting that form conversion had not occurred during the experiment (FIG. 7). Optical microscopy of the free form under cross polarized light showed birefringent irregular shaped particles (FIG. 12). Aqueous solubility was observed to be 2.0 mg/mL at pH 9.3 after 24 hours of equilibration with no change in crystalline form as assessed by XRPD (FIG. 7). Form A was confirmed to be stable after seven days of storage at 60° C. showing no signs of form conversion by XRPD. A summary of the results obtained from the characterization of Form A is presented in Table 7.

### Form B

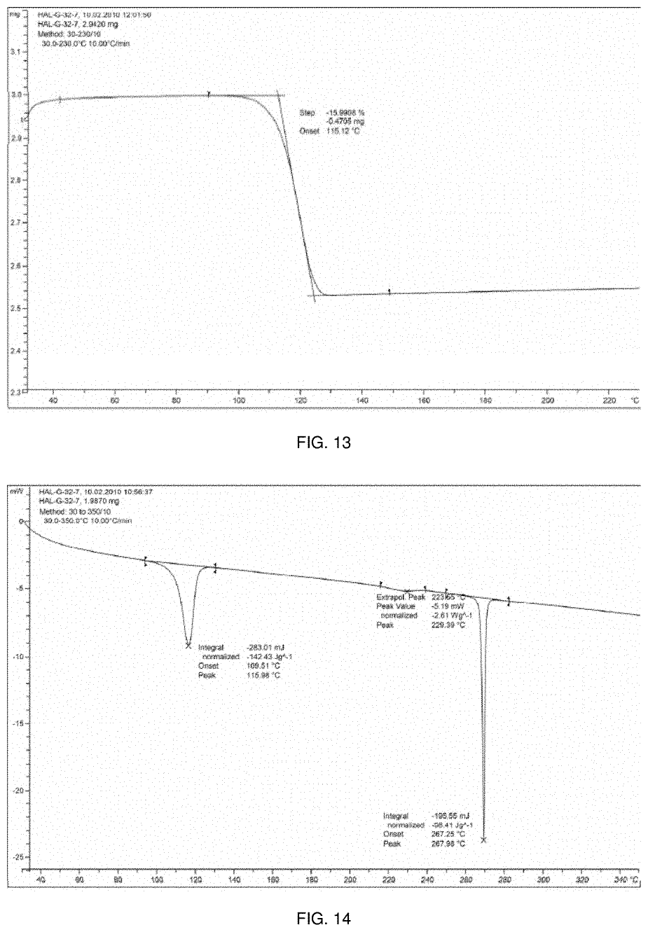

Lot HAL-G-32(7) obtained following a 7-day room temperature slurry of Form A in IPA, afforded a unique XRPD pattern which showed differences in comparison to the diffraction pattern of Form A (FIG. 7). Subsequent analysis by ¹H-NMR confirmed the chemical structure of formula (I) and showed approximately 15.7 wt % IPA (FIG. 9). TGA analysis showed a step transition from 90-130° C. equivalent to 16.0 wt % loss which is comparable to the theoretical IPA content (16.8 wt %) of a mono-IPA solvate of formula (I) (FIG. 13). Further thermal analysis by DSC showed two endothermic transitions at 116 and 268° C. attributed to desolvation and melting (FIG. 14). A summary of the results obtained from the characterization of Form B is presented in Table 7. The results obtained from the characterization of the formula (I) free form indicated the presence of two unique crystalline forms including an anhydrate (Form A) and mono-IPA solvate (Form B). Given that the melting temperature of Form A was determined to be 242° C., it can be further postulated that an additional un-isolated solid form of formula (I) may exist which melts at 268° C.

(2) Adipic acid

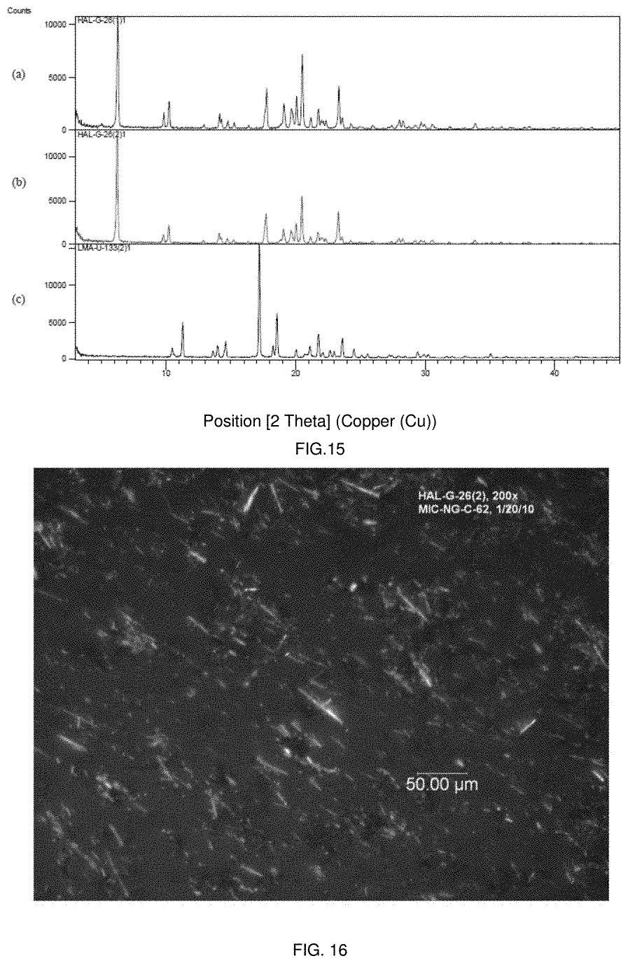

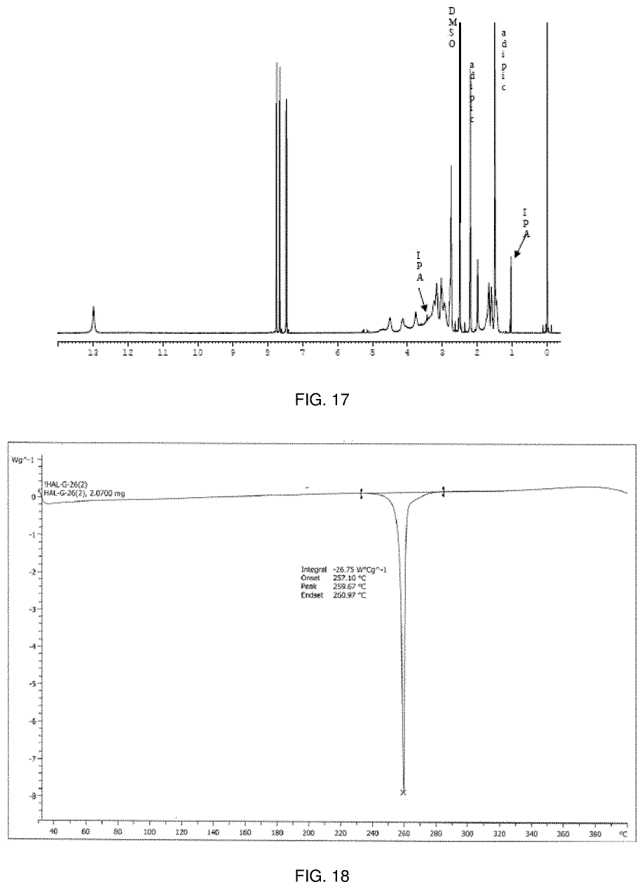

Lots HAL-G-26(1) isolated from EtOH and lot HAL-G-26(2) from IPA at a 70 mg scale, afforded the same crystalline XRPD pattern which was unique as compared to formula (I) free form and all other synthesized salts (FIG. 15). This solid form was also observed to be crystalline by optical microscopy exhibiting birefringent acicular particles under cross polarized light (FIG. 16). ¹H-NMR analysis of lot HAL-G-26(2) confirmed the chemical structure of formula (I) and showed an acid: API ratio of 0.5:1 indicating the formation of a hemi-adipate salt of formula (I) which was designated Form A. Further inspection of the ¹H-NMR spectrum showed approximately 0.4 wt % residual IPA (FIG. 17). Thermal analysis of Form A by DSC showed a single endothermic event at 260° C. which was attributed to melting of the crystalline salt (FIG. 18). No weight loss was observed by TGA. The solubility of the Form A hemi-salt in water was estimated to be in excess of 400 mg/mL by visual inspection as complete dissolution was observed. While this salt showed desirable physical characteristics, it was not selected for further characterization due to the counter ion's lack of prevalence in pharmaceutical drug products. A summary of the results obtained from the characterization of Form A is presented in Table 7.

### (3) Benzenesulfonic Acid

### Form A

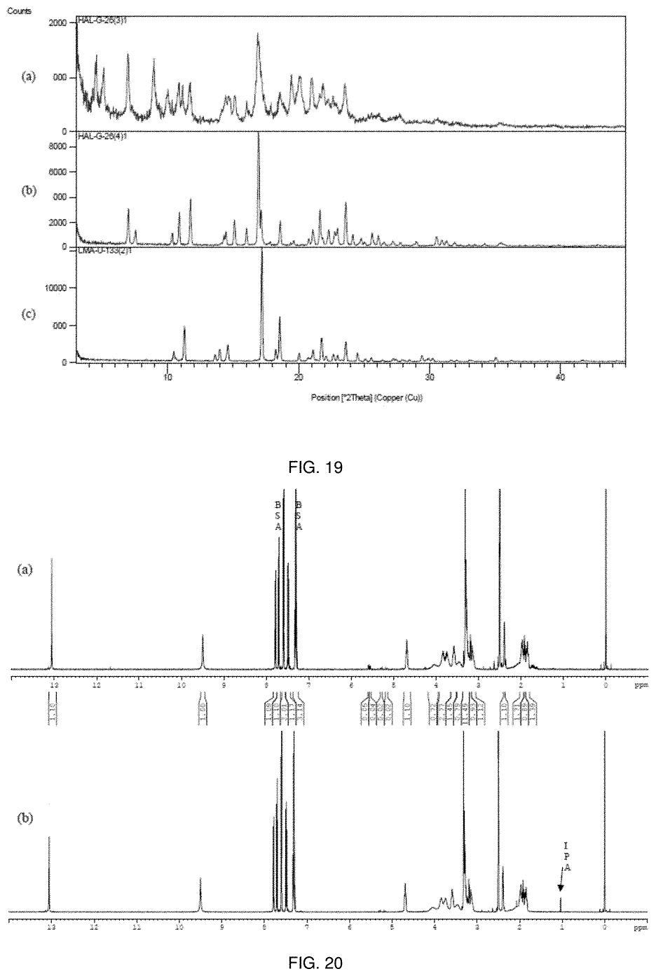

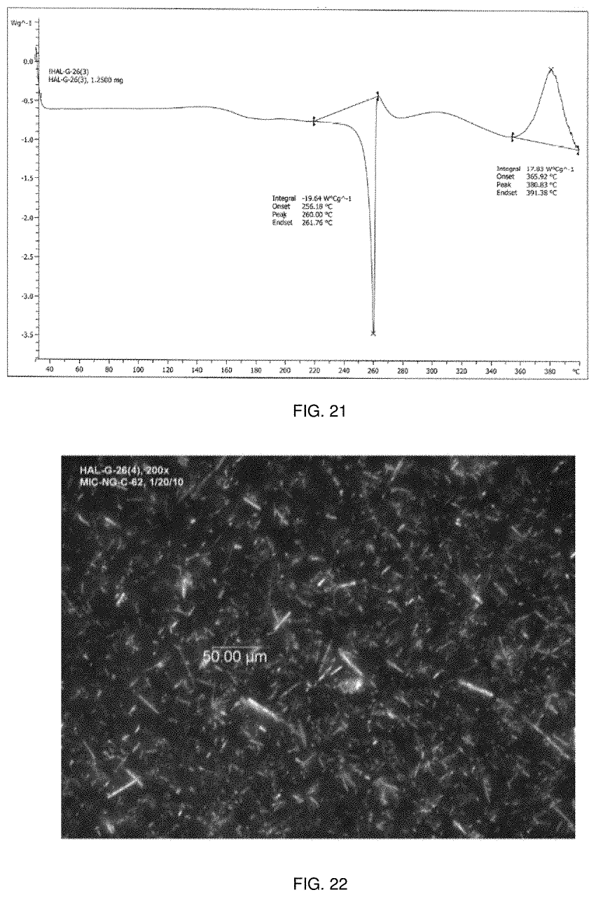

Lots HAL-G-26(3) isolated from THE at a 70 mg scale, afforded a crystalline XRPD pattern which was unique in comparison to the diffraction pattern of formula (I) free form and all other synthesized salts (FIG. 19). This solid form was also observed to be crystalline under cross polarized light. ¹H-NMR analysis of lot HAL-G-26(3) confirmed the chemical structure of formula (I) and showed an acid: API ratio of 1.0:1 indicating the formation of a mono-besylate salt of formula (I) which was designated Form A (FIG. 20). DSC analysis of Form A showed an endothermic event at 260° C. attributed to melting followed by decomposition at 381° C. (FIG. 21). No weight loss was observed by TGA. The solubility of the Form A mono-salt in water was estimated to be in excess of 400 mg/mL by visual inspection as complete dissolution was observed. A summary of the results obtained from the characterization of Form A is presented in Table 7.

### Form B

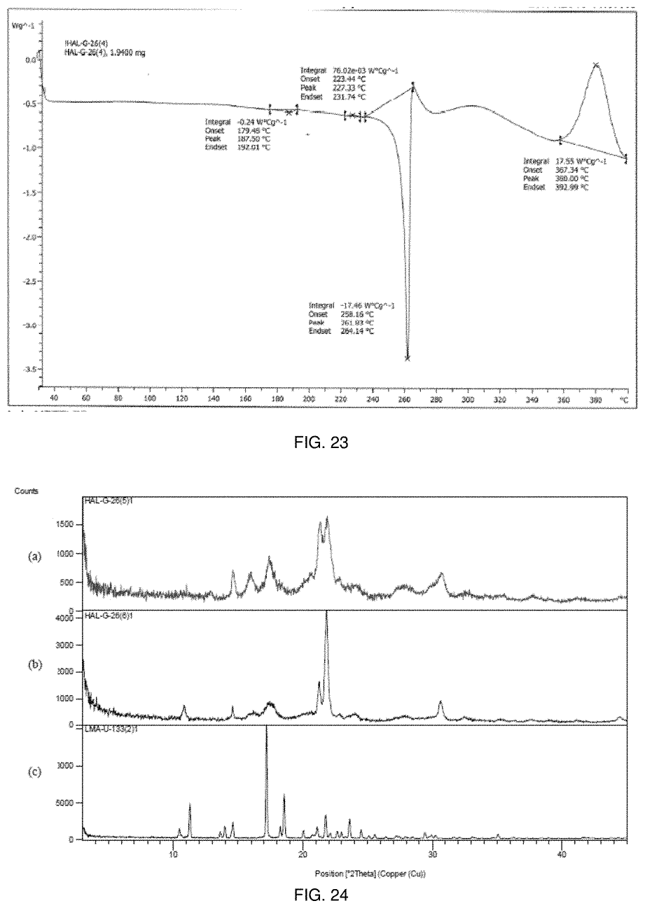

Lots HAL-G-26(4) isolated from IPA at a 70 mg scale, afforded a crystalline XRPD pattern which was unique in comparison to the diffraction pattern of formula (I) free form and the Form A besylate salt (FIG. 19). This solid form was also observed to be crystalline under cross polarized light (FIG. 22). ¹H-NMR analysis of lot HAL-G-26(4) confirmed the chemical structure of formula (I) and showed an acid: API ratio of 1.0:1 confirming the formation of a mono-besylate salt of formula (I) which was designated Form B. Further inspection of the ¹H-NMR spectrum showed approximately 0.1 wt % residual IPA (FIG. 20). TGA analysis of Form B showed no weight loss. Further analysis of Form B by DSC showed minor transitions at 188° C. and 227° C. followed by melting 262° C. (FIG. 23). The solubility of the Form B mono-salt in water was estimated to be in excess of 400 mg/mL by visual inspection as complete dissolution was observed.

The results obtained from the characterization of the formula (I) besylate indicated the presence of two crystalline mono-salt forms (Forms A and B). This counter ion was not selected for further characterization due to additional testing requirements which would be required for genotoxic impurities if isolated from an alcoholic solvent. A summary of the results obtained from the characterization of Form B is presented in Table 7.

### (4) Hydrobromic Acid

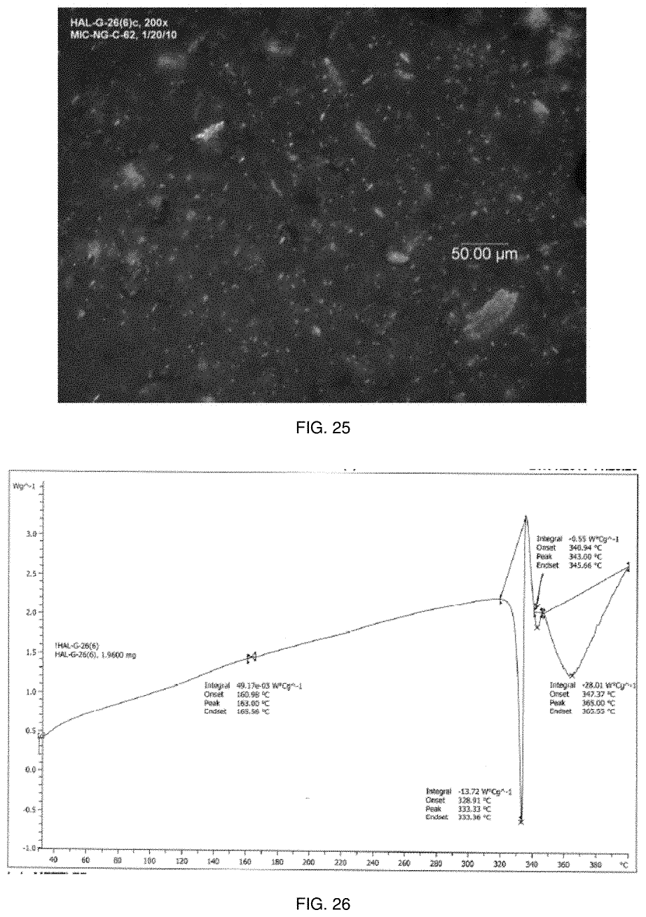

Lots HAL-G-26(5) isolated from THF and lot HAL-G-26(6) from IPA at a 70 mg scale, afforded the same semi-crystalline XRPD pattern which was unique as compared to formula (I) free form and all other synthesized salts (FIG. 24). This solid form also showed evidence of crystallinity under cross polarized light (FIG. 25). The ¹H-NMR spectrum of lot HAL-G-26(6) was consistent with the chemical structure of formula (I) and showed approximately 0.5 wt % residual IPA. Mono-salt formation was confirmed by ICP/OES. Thermal analysis of the bromide salt by DSC showed a single endothermic event at 333° C. which was attributed to melting of the crystalline salt (FIG. 26). No weight loss was observed by TGA. The solubility of the bromide salt in water was estimated to be 56.1 mg/mL using a TGA gravimetric method. While this salt showed desirable aqueous solubility, it was not selected due to its lack of crystallinity and general safety concerns associated with hydrobromide salts. A summary of the results obtained from the characterization of Form A is presented in Table 7.

(5) Fumaric acid

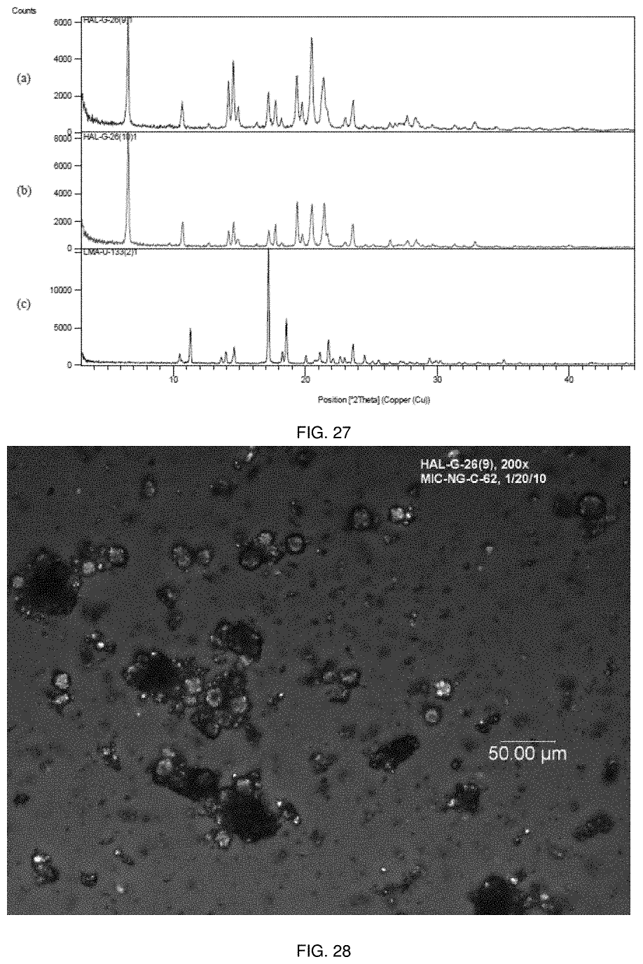

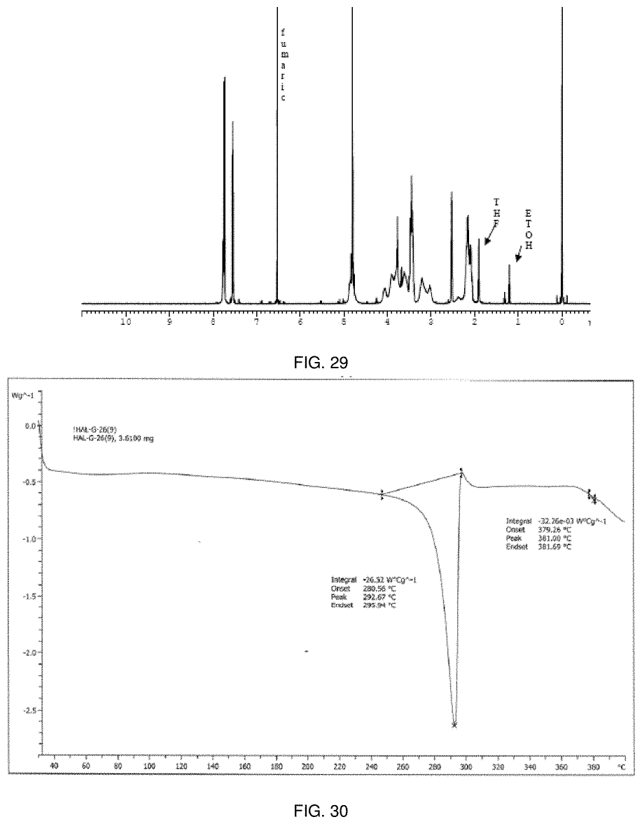

Lots HAL-G-26(9) isolated from THF and lot HAL-G-26(10) from IPA at a 70 mg scale, afforded the same crystalline XRPD pattern which was unique as compared to formula (I) free form and all other synthesized salts (FIG. 27). Examination of the solid under cross polarized light revealed birefringent particles with an irregular shaped crystal habit (FIG. 28). ¹H-NMR analysis of lot HAL-G-26(9) confirmed the chemical structure of formula (I) and showed an acid: API ratio of 1.0:1 indicating the formation of a monofumarate salt of formula (I) which was designated Form A. Further inspection of the ¹H-NMR spectrum showed approximately 1.6 wt % THF and 0.4 wt % EtOH (FIG. 29). Thermal analysis of the fumarate salt by DSC showed a single endothermic event at 293° C. which was attributed to melting of the crystalline salt (FIG. 30). No weight loss was observed by TGA. The solubility of Form A in water was estimated to be 400 mg/mL by visual inspection as complete dissolution was observed. This salt was not selected for further characterization due to the potential for isomerization to maleic acid which has been responsible for causing undesirable toxicological effects in dogs. A summary of the results obtained from the characterization of Form A is presented in Table 7.

### (6) Benzoic Acid

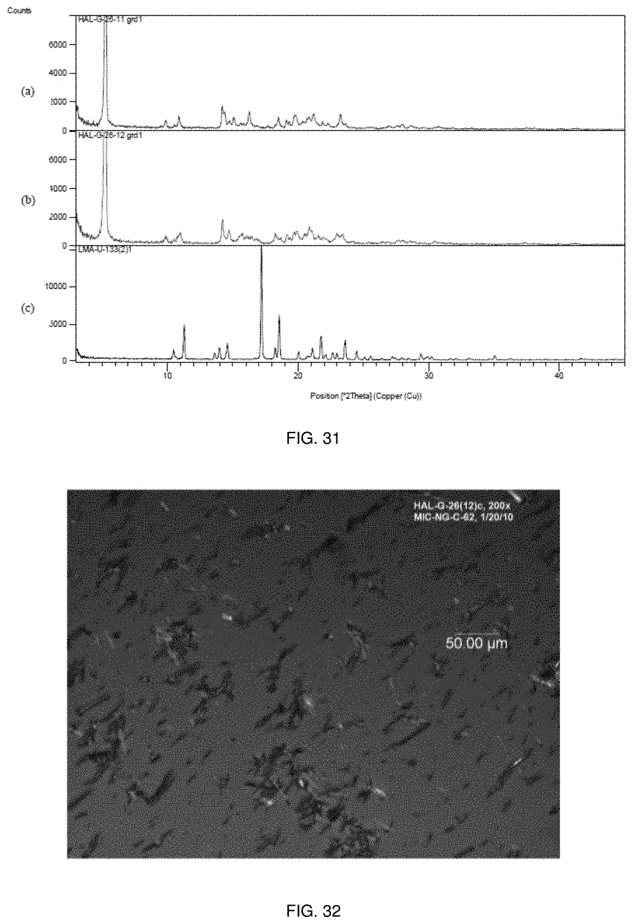

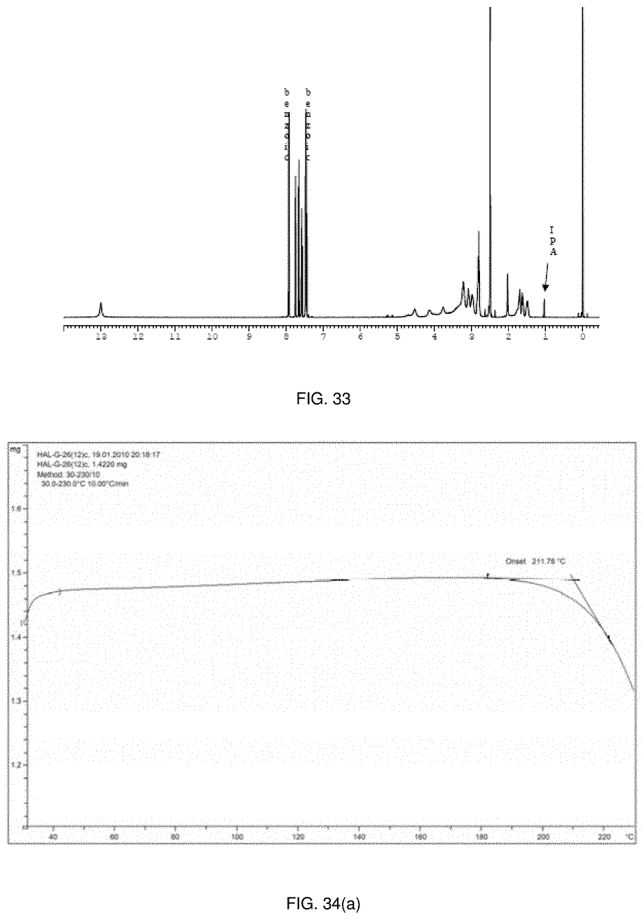

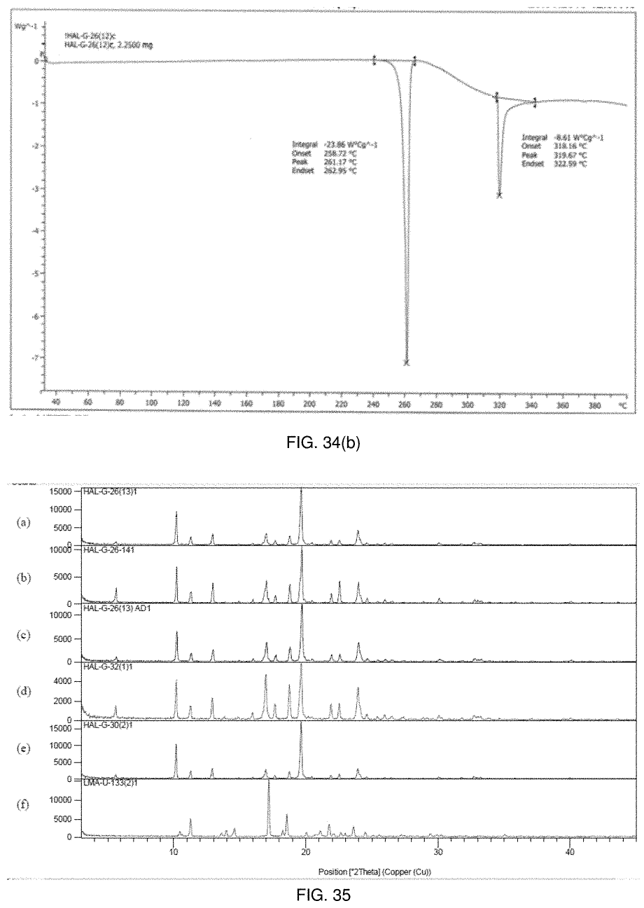

Lots HAL-G-26(11) isolated from THF and lot HAL-G-26(12) from IPA at a 70 mg scale, afforded the same crystalline XRPD pattern which was unique as compared to formula (I) free form and all other synthesized salts (FIG. 31). Examination of the solid under cross polarized light revealed birefringent particles with acicular and blade like crystal habits (FIG. 32). ¹H-NMR analysis of lot HAL-G-26(12) confirmed the chemical structure of formula (I) and showed an acid: API ratio of 1.0:1 indicating the formation of a monobenzoate salt of formula (I) which was designated Form A (FIG. 33). TGA analysis showed no weight loss while two endothermic transitions at 261° C. and 320° C. were observed by DSC attributed to melting and decomposition (FIG. 34). The solubility of the benzoate salt in water was estimated to be 12.5 mg/mL using a TGA gravimetric method. While Form A was observed to be crystalline and showed improved solubility over the free form, it was not selected due to lack of prevalence in pharmaceutical drug products and general safety concerns associated with benzoate salts. A summary of the results obtained from the characterization of Form A is presented in Table 7.

### (7) Methanesulfonic Acid

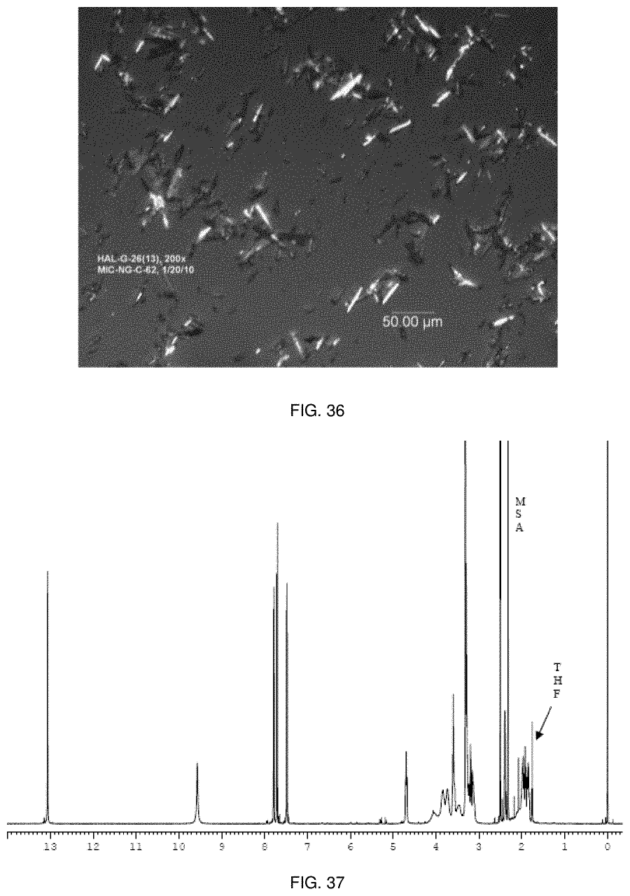

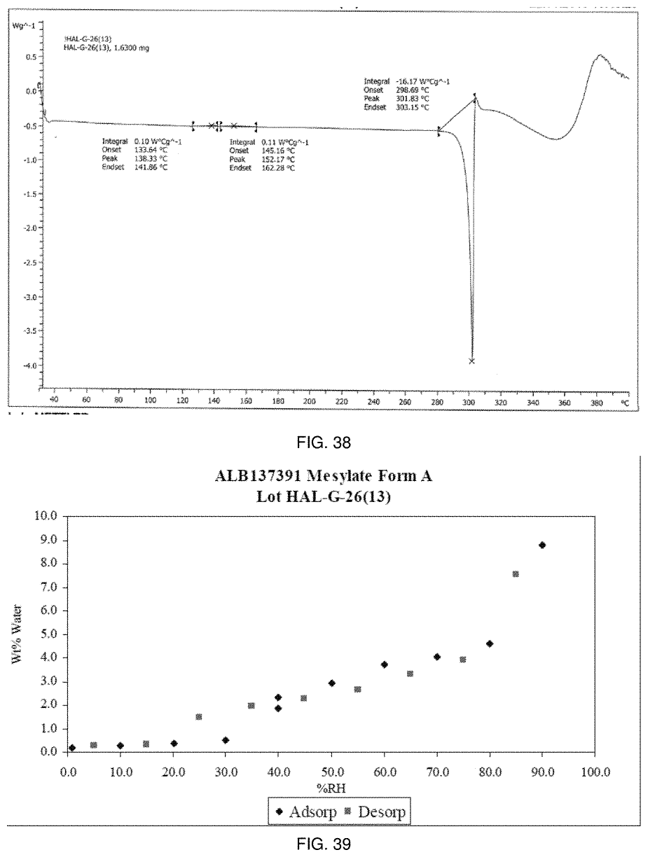

Lots HAL-G-26(13) isolated from THF and lot HAL-G-26(14) from IPA at a 70 mg scale, afforded consistent crystalline XRPD patterns which showed differences in comparison to the diffraction pattern of the formula (I) free form (FIG. 35). Examination of lot HAL-G-26(13) under cross polarized light revealed birefringent particles with acicular and blade like morphologies (FIG. 36). ¹H-NMR analysis of lot HAL-G-26(13) confirmed the chemical structure of formula (I) and showed an acid: API ratio of 1.0:1 indicating the formation of a mono-mesylate salt of formula (I) which was designated Form A (FIG. 37). Thermal analysis of the mesylate salt by DSC showed a single endothermic event at 302° C. which was attributed to melting of the crystalline salt (FIG. 38). No weight loss was observed by TGA. The solubility of Form A in water was estimated to be 400 mg/mL by visual inspection as complete dissolution was observed. This salt was selected for full characterization based upon thermal stability, high degree of crystallinity, and improved aqueous solubility observed in comparison to the free form.

Form A was observed to be moderately hygroscopic by gravimetric moisture sorption adsorbing up to 4.6 wt % water at 80% RH followed by a sharp increase to 8.8 wt % at 90% RH (FIG. 39). XRPD analysis of the dried material following the program afforded a crystalline pattern that was consistent with the starting material, suggesting no form conversion had occurred during the experiment (FIG. 35). Form A was observed to be stable showing no signs of conversion following seven days of room temperature equilibration in THF and storage at 60° C. (FIG. 35). These findings suggest that Form A is the thermodynamically favored crystalline form of formula (I) mono-mesylate salt. A summary of the results obtained from the characterization of Form A is presented in Table 7.

### (8) L-Malic Acid

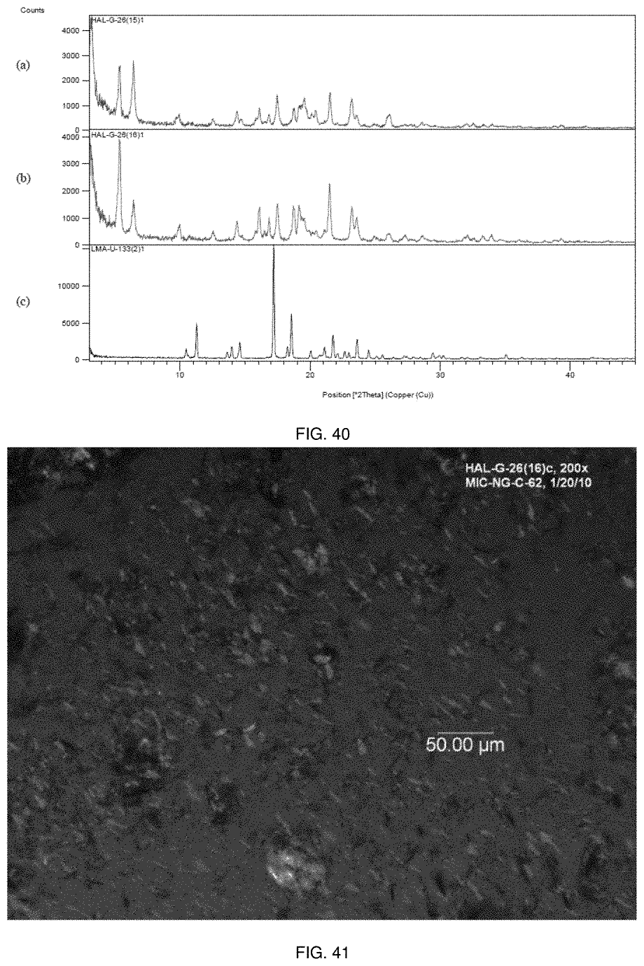

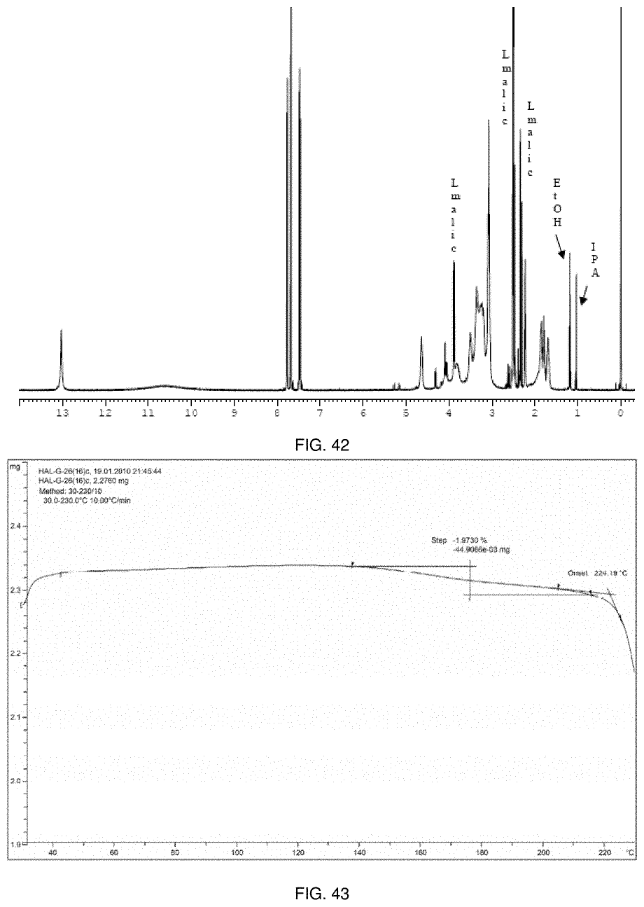

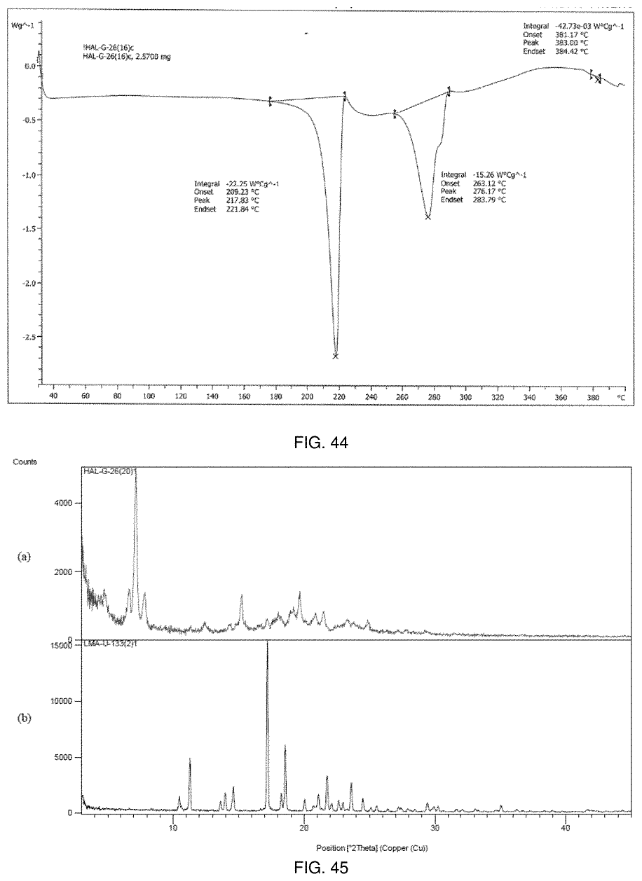

Lots HAL-G-26(15) isolated from THE and lot HAL-G-26(16) from IPA at a 70 mg scale, afforded the same crystalline XRPD pattern which was unique as compared to formula (I) free form and all other synthesized salts (FIG. 40). Examination of the solid under cross polarized light provided further evidence of crystallinity (FIG. 41). ¹H-NMR analysis of lot HAL-G-26(16) confirmed the chemical structure of formula (I) and showed an acid: API ratio of 0.4:1 suggesting the formation of a hemi-malate salt of formula (I) which was designated Form A (FIG. 42). Further inspection of the ¹HNMR spectrum showed approximately 0.4 wt % IPA and 1.1 wt % EtOH (FIG. 42). TGA analysis showed a 2.0 wt % loss between 140° C. and 200° C. attributed to loss of IPA and EtOH (FIG. 43). Two endothermic transitions were observed by DSC at 218° C. and 276° C. which were attributed melting and decomposition of the salt (FIG. 44). The solubility of the malate salt in water was estimated to be 80 mg/mL by visual inspection as complete dissolution was observed. While this salt showed desirable physical characteristics, it was not selected for further characterization due to its limited thermal stability in comparison to the free form. A summary of the results obtained from the characterization of Form A is presented in Table 7.

### (9) Sorbic Acid

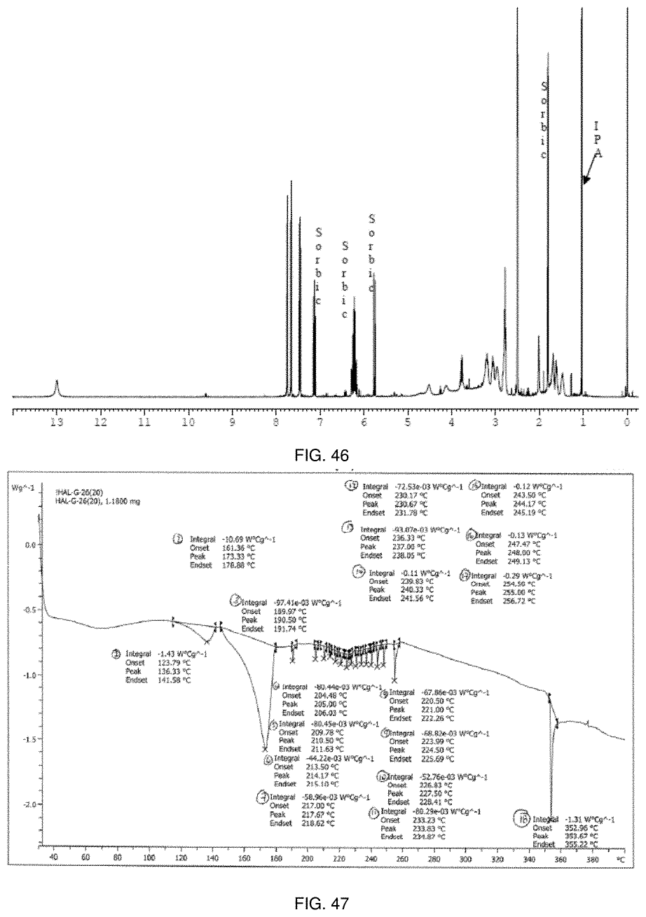

Lots HAL-G-26(20) isolated from IPA at a 70 mg scale, afforded a crystalline XRPD pattern which was unique as compared to formula (I) free form and all other synthesized salts (FIG. 45). Examination of the solid under cross polarized light provided further evidence of crystallinity. ¹H-NMR analysis of lot HAL-G-26(20) showed approximately 2.3 wt % IPA and an acid: API ratio of 0.9:1 suggesting the formation of a mono-sorbate salt of formula (I) which was designated Form A. Further inspection of the ¹H-NMR spectrum showed several impurity resonances at 1.3, 1.9, 2.4, 3.6, and 4.3 ppm (FIG. 46). TGA analysis showed continuous weight loss across the temperature program from 30° C.-230° C. Further testing by DSC showed two major endotherms at 136° C. and 173° C. and several minor transitions which may be attributed to the presence of impurities and/or instrumental artifacts (FIG. 47). The solubility of Form A in water was estimated to be 200 mg/mL by visual inspection as complete dissolution was observed. This salt was not selected for further characterization due to impurities observed during the analysis and the counter ion's lack of prevalence in pharmaceutical drug products. A summary of the results obtained from the characterization of Form A is presented in Table 7.

### (10) Phosphoric Acid

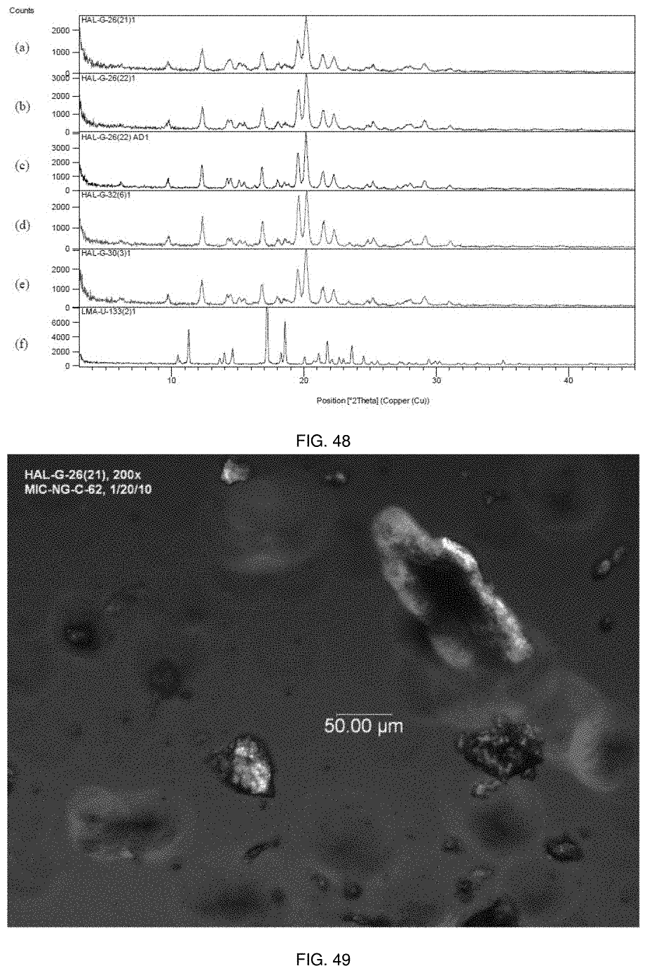

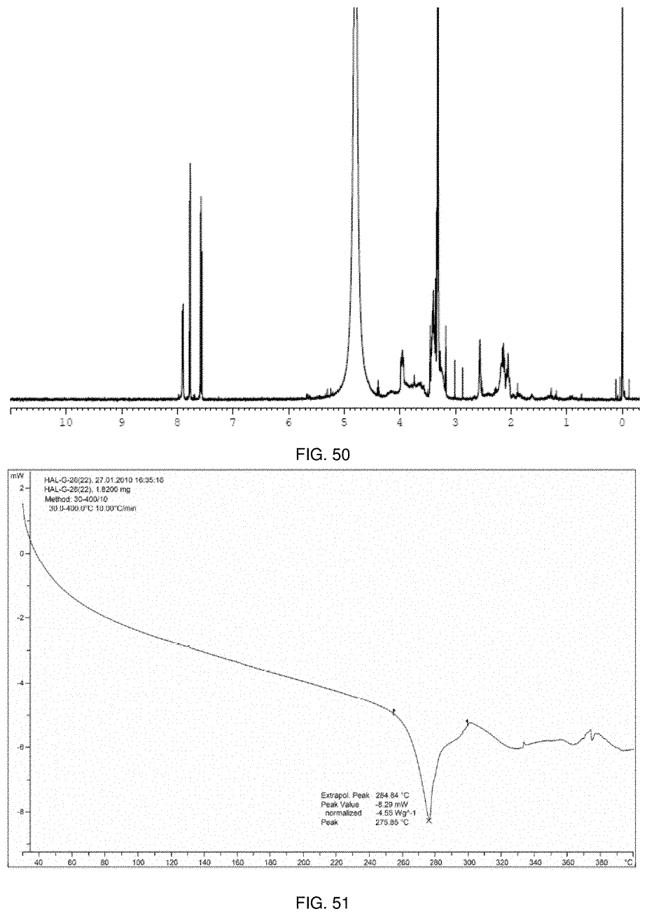

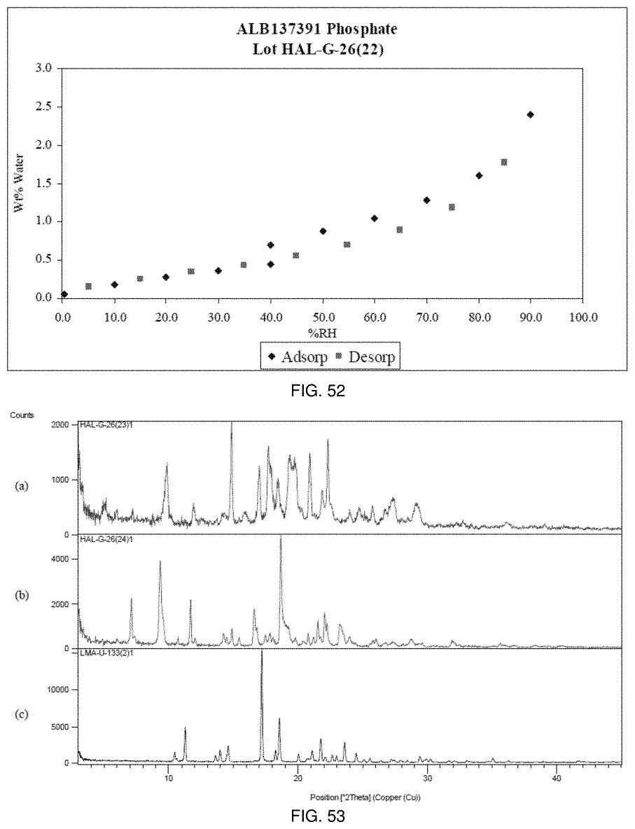

Lots HAL-G-26(21) isolated from THE and lot HAL-G-26(22) from IPA at a 70 mg scale, afforded consistent crystalline XRPD patterns (FIG. 48). The crystallinity of this unique solid was confirmed by examination under cross polarized light (FIG. 49). The ¹H-NMR spectrum of lot HAL-G-26(21) was consistent with the chemical structure of formula (I) and showed no residual solvents (FIG. 50). Mono-salt formation was confirmed by ICP/OES. Thermal analysis of lot HAL-G-26(22) by DSC showed a single endothermic event at 276° C. which was attributed to melting of the crystalline salt (FIG. 51) and no weight loss was observed by TGA. The solubility of Form A in water was determined to be 52 mg/mL by HPLC following an overnight room temperature water slurry. Given the improved thermal stability and aqueous solubility achieved with Form A in comparison to the free form, this salt was selected for full characterization. Form A was observed to be slightly hygroscopic adsorbing up to 1.1 wt % water at 60% RH and 2.4 wt % water at 90% RH (FIG. 52). XRPD analysis of the dried material following the program afforded a crystalline pattern that was consistent with the starting material, suggesting no form conversion had occurred during the experiment (FIG. 48). Form A was observed to be stable showing no signs of conversion following seven days of room temperature equilibration in IPA and storage at 60° C. (FIG. 48). These findings suggest that Form A is the thermodynamically favored crystalline form of formula (I) mono-phosphate salt. A summary of the results obtained from the characterization of Form A is presented in Table 7.

### (11) Sulfuric Acid

### Form A

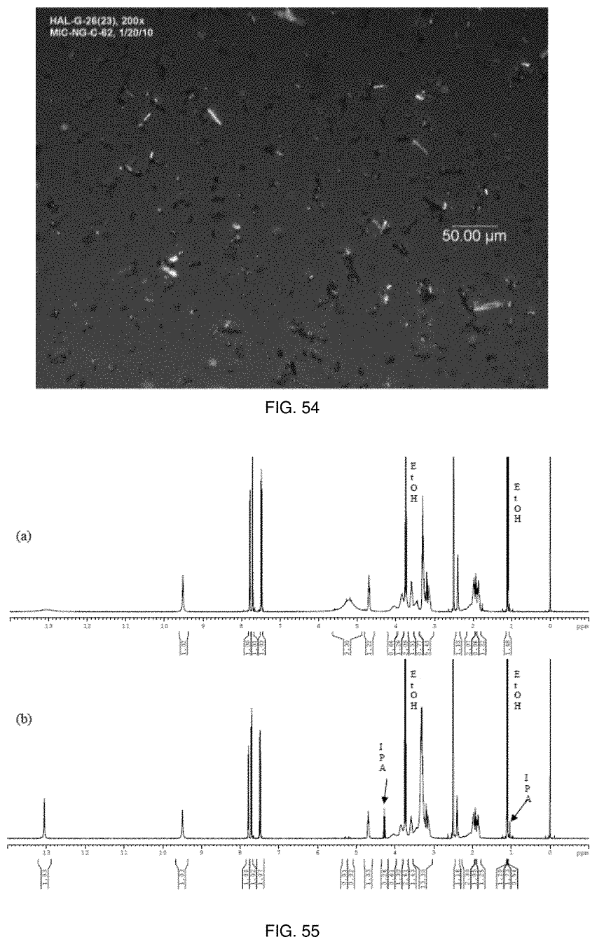

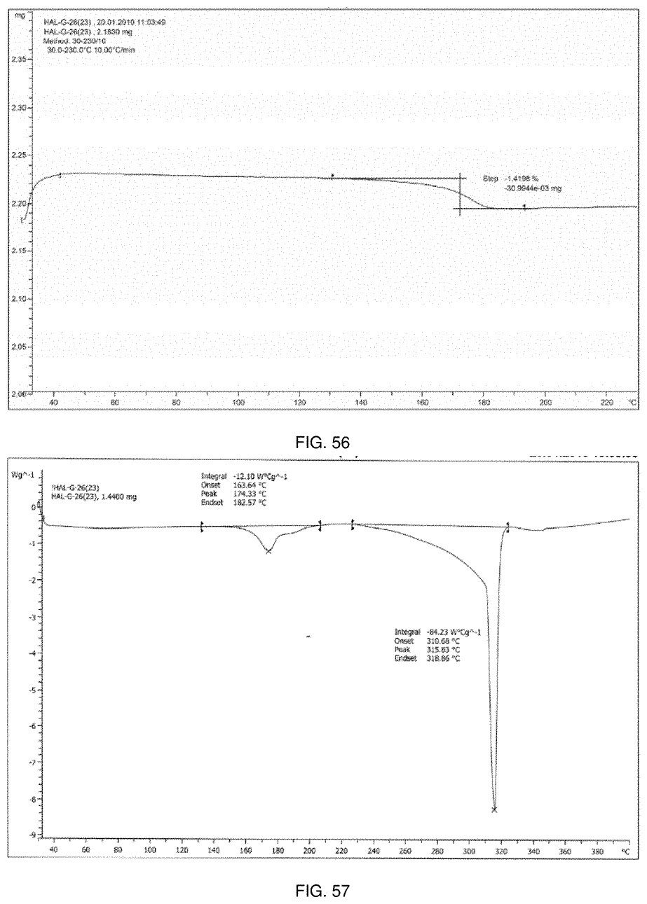

Lots HAL-G-26(23) isolated from THF at a 70 mg scale, afforded a crystalline XRPD pattern which was unique in comparison to the diffraction pattern of formula (I) free form and all other synthesized salts (FIG. 53). This solid form exhibited birefringence acicular particles under cross polarized light (FIG. 54). The ¹H-NMR spectrum of lot HAL-G-26(23) was consistent with the chemical structure of formula (I) and showed approximately 5.4 wt % EtOH (FIG. 55). Mono-salt formation was confirmed by ICP/OES and the solubility of the salt in water was estimated to be 18.9 mg/mL. TGA analysis showed a step transition from 140° C.-190° C. likely attributed to bound weight loss of 1.4 wt % water or EtOH (FIG. 56). The DSC thermogram of Form A showed an endothermic transition at 174° C. corresponding to the weight loss event observed by TGA followed by a melting event at 316° C. (FIG. 57). In an effort to elucidate the transition at 174° C., an additional DSC experiment was performed where the sample was heated twice to 210° C. to ensure completion of the initial endothermic event. Subsequent analysis of the sample by XRPD revealed conversion to an amorphous solid and the corresponding ¹H-NMR spectrum showed signs of degradation. Given these findings, further investigation would be required to determine if the salt was solvated with water or EtOH. A summary of the results obtained from the characterization of Form A is presented in Table 7.

### Form B

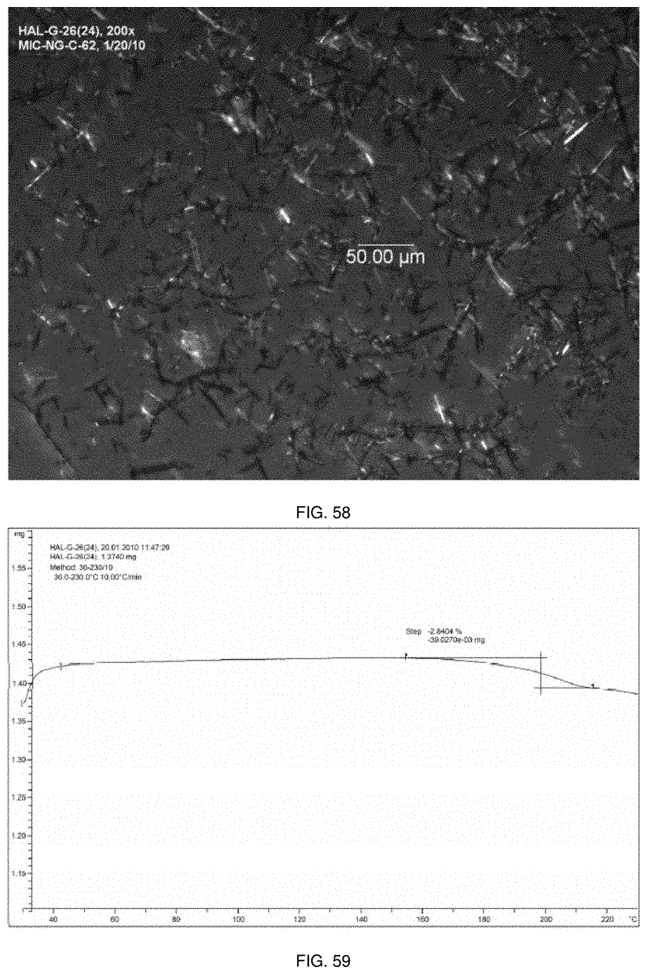

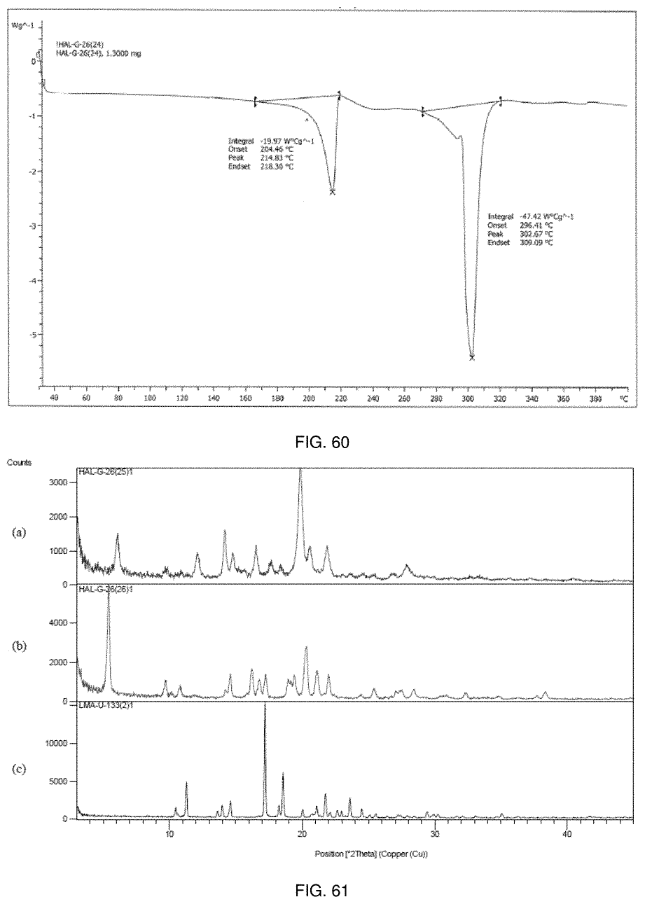

Lots HAL-G-26(24) isolated from IPA at a 70 mg scale, afforded a crystalline XRPD pattern which was unique in comparison to the diffraction pattern of formula (I) free form and the Form A sulfate salt (FIG. 53). The crystallinity of this solid form was confirmed by optical microscopy (FIG. 58). The ¹H-NMR spectrum of lot HAL-G-26(24) was consistent with the chemical structure of formula (I) and showed approximately 1.4 wt % IPA and 6.3 wt % EtOH (FIG. 55). Mono-salt formation was confirmed by ICP/OES and the solubility of the salt in water was estimated to be 69.2 mg/mL. TGA analysis showed a step transition from 160° C.-220° C. likely attributed to bound weight loss of 2.8 wt % water or EtOH (FIG. 59). The DSC thermogram of Form B showed an endothermic transition at 215° C. corresponding to the weight loss event observed by TGA followed by a melting event at 303° C. (FIG. 60). In an effort to elucidate the transition at 215° C., an additional DSC experiment was performed where the sample was heated twice to 240° C. to ensure completion of the initial endothermic event.

Subsequent analysis of the sample by XRPD revealed conversion to an amorphous solid and the corresponding ¹H-NMR spectrum showed signs of degradation. Given these findings, further investigation would be required to determine if the salt is solvated with water or EtOH. The results obtained from the characterization of the formula (I) sulfate indicated the presence of two crystalline mono-salt forms (Forms A and B). This counter ion was not selected for further characterization due to the potential for solvate formation. A summary of the results obtained from the characterization of Form B is presented in Table 7.

### (12) L-Tartaric Acid

### Form a

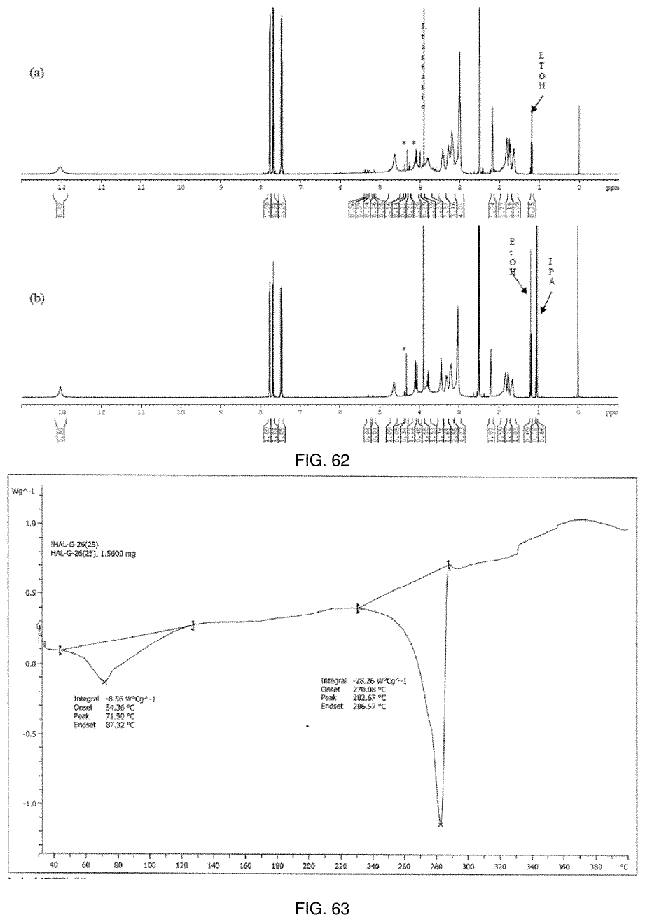

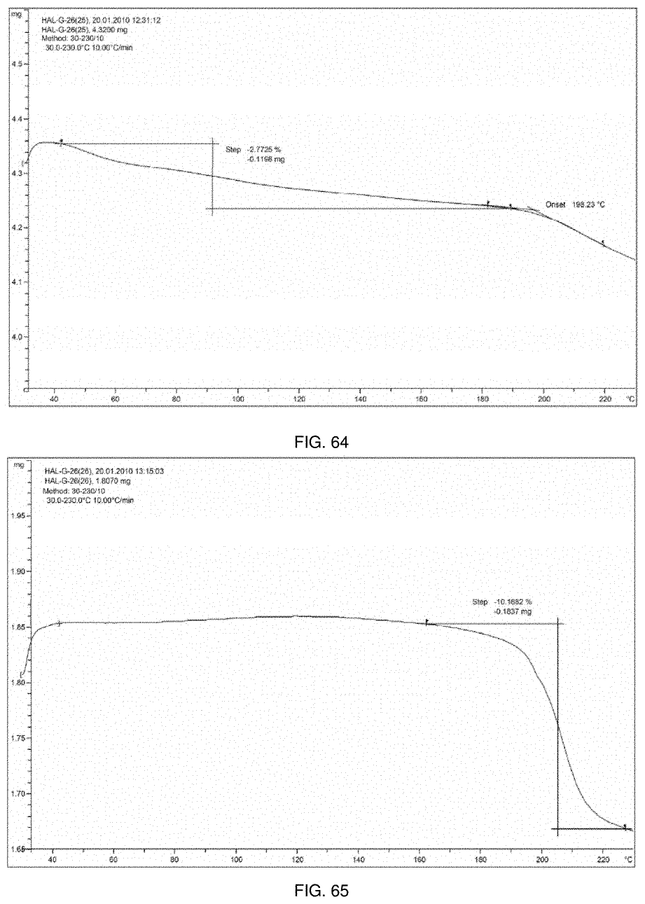

Lots HAL-G-26(25) isolated from THE at a 70 mg scale, afforded a crystalline XRPD pattern which was unique as compared to formula (I) free form and all other synthesized salts (FIG. 61). The crystallinity of the sample was also confirmed by examination under cross polarized light. The stoichiometry of this solid was estimated to be 0.5:1 by ¹H-NMR due to overlapping API resonances with L-tartaric acid (FIG. 62). These findings suggest the formation of a hemi-tartrate salt of formula (I) which was designated Form A. Further inspection of the ¹H-NMR spectrum showed approximately 1.0 wt % EtOH and minor impurity resonances (FIG. 62). Thermal analysis of the tartrate salt by DSC showed a broad endotherm at 72° C. attributed to liberation of EtOH followed by an endotherm at 283° C. due to melting and decomposition (FIG. 63). TGA analysis showed 2.8 wt % loss attributed to loss of EtOH (FIG. 64). The solubility of Form A in water was estimated to be 90 mg/mL by visual inspection as complete dissolution was observed. A summary of the results obtained from the characterization of Form A is presented in Table 7.

### Form B

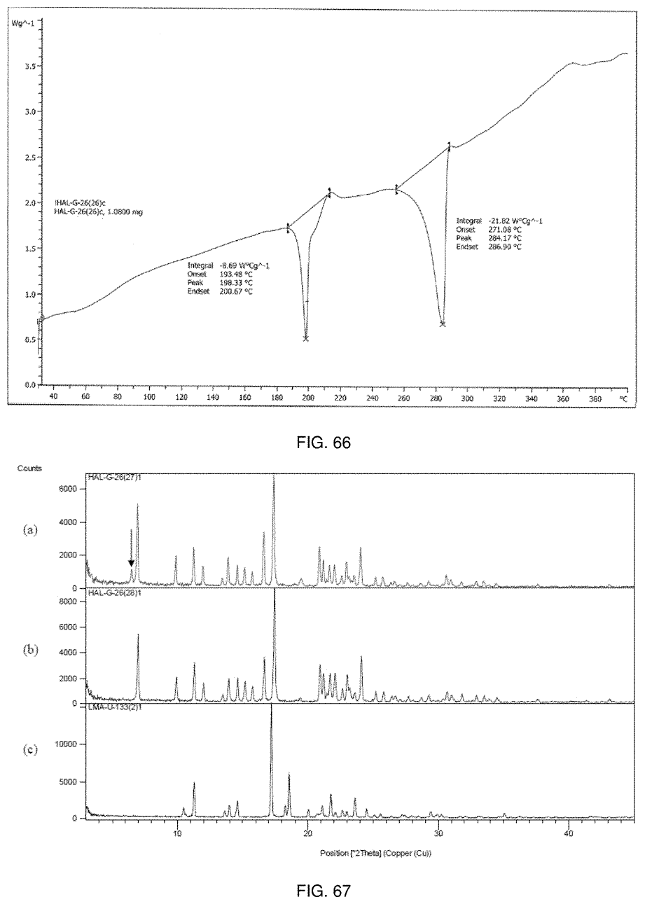

Lots HAL-G-26(26) isolated from IPA at a 70 mg scale, afforded a crystalline XRPD pattern which was unique as compared to formula (I) free form and the Form A tartrate salt (FIG. 61). The crystallinity of the sample was also confirmed by examination under cross polarized light. The stoichiometry of this solid was estimated to be 0.5:1 by ¹H-NMR due to overlapping API resonances with L-tartaric acid. These findings suggest the formation of a hemi-tartrate salt of formula (I) which was designated Form B. Further inspection of the ¹H-NMR spectrum showed approximately 2.3 wt % IPA and 0.5 wt % EtOH (FIG. 62). TGA analysis showed a step transition between 160° C. and 220° C. attributed to the loss of 10.2 wt % water (FIG. 65). This result is comparable to the theoretical water content of a di-hydrate formula (I) hemi-tartrate salt which is 9.2 wt %. Two endothermic transitions were observed by DSC at 198° C. and 284° C. which were attributed to dehydration and melting of the salt (FIG. 66). The solubility of the Form B tartrate salt in water was estimated to be 120 mg/mL by visual inspection as complete dissolution was observed. The results obtained from the characterization of formula (I) tartrate indicated the presence of a crystalline anhydrate and a hydrate hemi-salt form.

This counter ion was not selected for further characterization due to the potential for hydrate formation which can often present stability challenges during long term storage. A summary of the results obtained from the characterization of Form B is presented in Table 7.

(13) p-Toluenesulfonic Acid

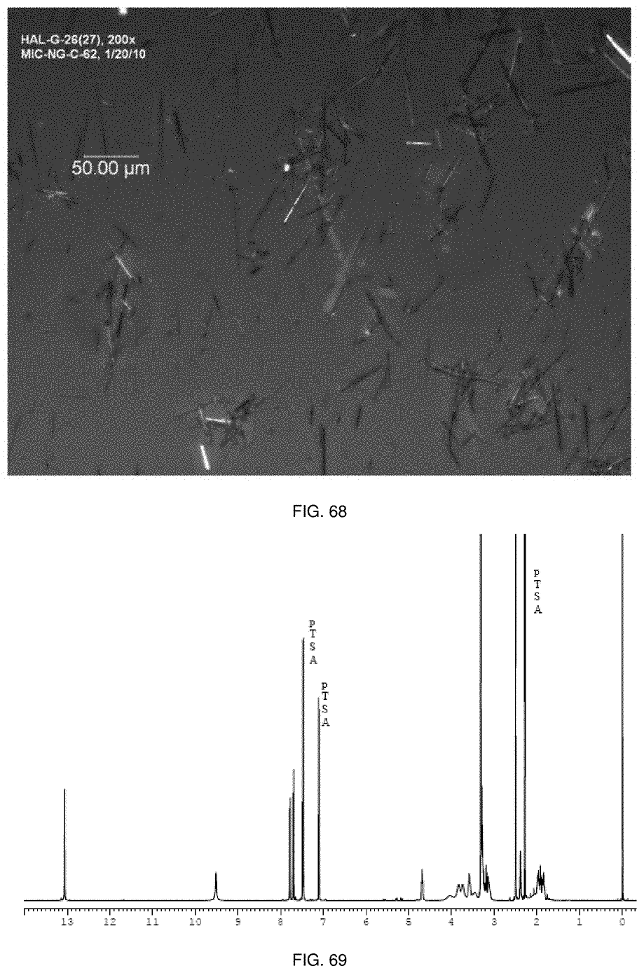

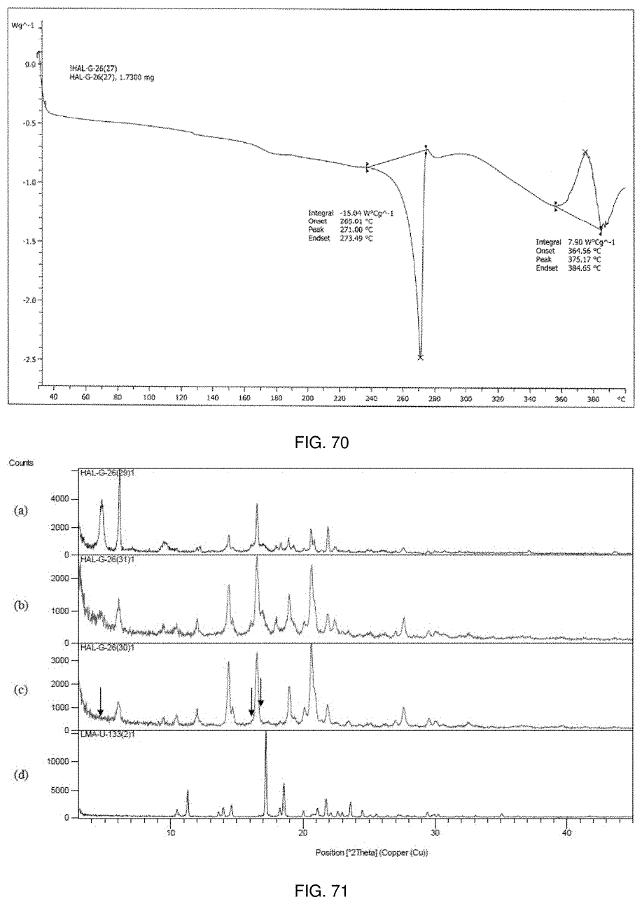

Lots HAL-G-26(27) isolated from THE and lot HAL-G-26(28) from IPA at a 70 mg scale, afforded similar crystalline XRPD patterns with the exception of an additional minor reflection at 6.5 degrees 2-theta (FIG. 67). Examination of lot HAL-G-26(27) under cross polarized light revealed birefringent particles with acicular and blade like morphologies (FIG. 68). ¹H-NMR analysis of lot HAL-G-26(27) confirmed the chemical structure of formula (I) and showed an acid: API ratio of 1.0:1 indicating the formation of a monotosylate salt of formula (I) which was designated Form A (FIG. 69). Thermal analysis of the tosylate salt by DSC showed a single endothermic event at 271° C. which was attributed to melting of the crystalline salt (FIG. 70) and no weight loss was observed by TGA. The solubility of Form A in water was estimated to be 20.3 mg/mL by a TGA gravimetric method. This salt was not selected for further characterization due to additional testing requirements which would be required for genotoxic impurities if isolated from an alcoholic solvent. A summary of the results obtained from the characterization of Form A is presented in Table 7.

### (14) Citric Acid

### Form A

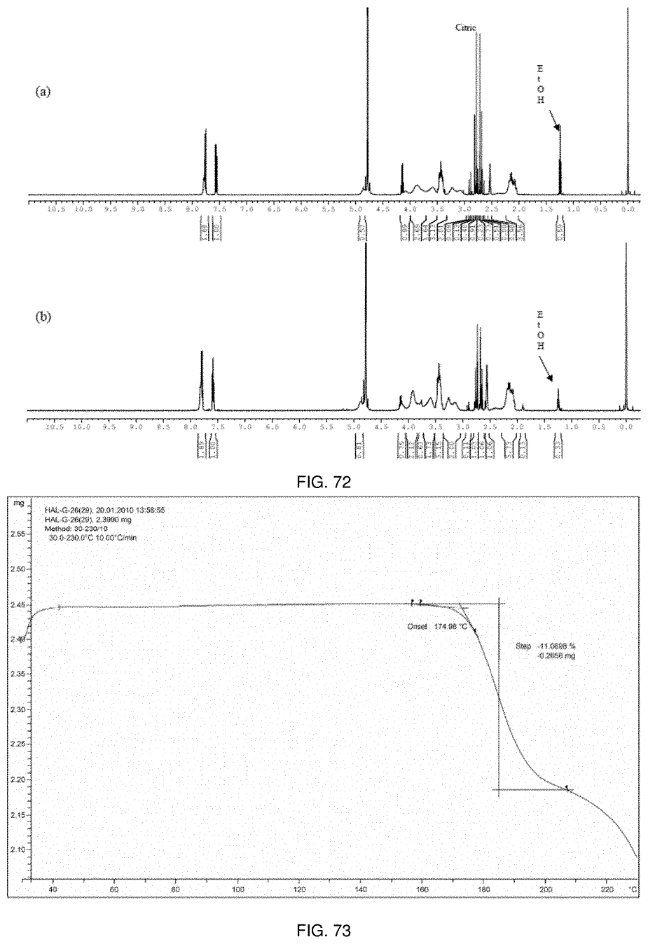

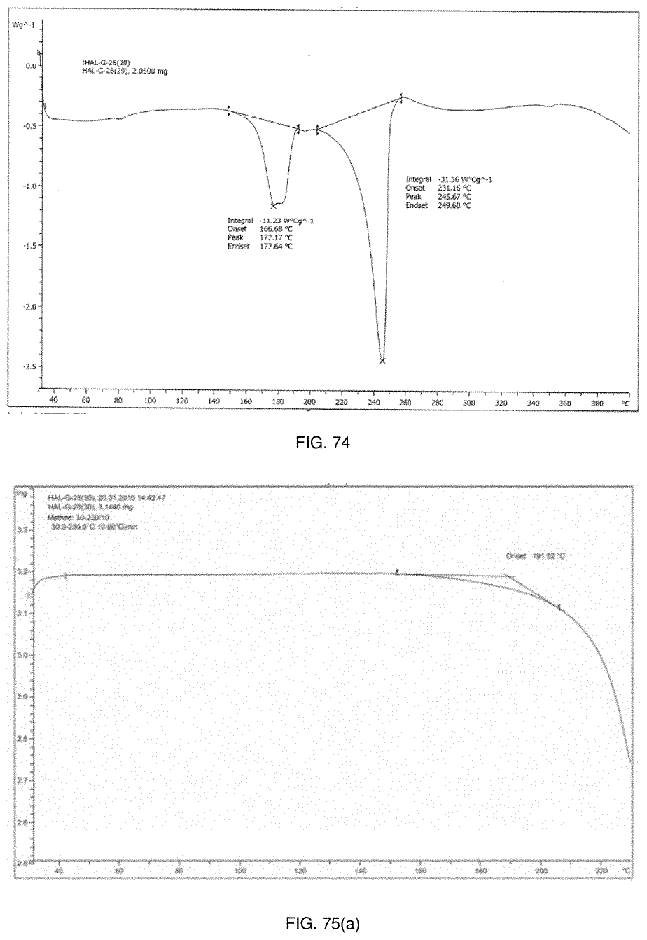

Lots HAL-G-26(29) isolated from MeCOH and lot HAL-G-26(31) from IPA at a 70 mg scale, afforded the same crystalline XRPD pattern which was unique as compared to formula (I) free form and all other synthesized salts (FIG. 71). The crystallinity of the solid was also confirmed by examination under cross polarized light. ¹H-NMR analysis showed an acid:API ratio of 0.5:1 indicating the formation of a hemi-salt. Further inspection of the ¹H-NMR spectrum showed approximately 2.1 wt % EtOH (FIG. 72). TGA analysis showed a step transition between 150° C. and 210° C. attributed to the loss of 11.1 wt % water (FIG. 73). This result is comparable to the theoretical water content of dehydrate formula (I) hemi-citrate salt which is 8.8 wt %. Two endothermic transitions were observed by DSC at 177° C. and 246° C. which were attributed to dehydration and melting of the salt (FIG. 74). The solubility of Form A in water was estimated to be 54.0 mg/mL by a TGA gravimetric method. A summary of the results obtained from the characterization of Form A is presented in Table 7.

### Form B

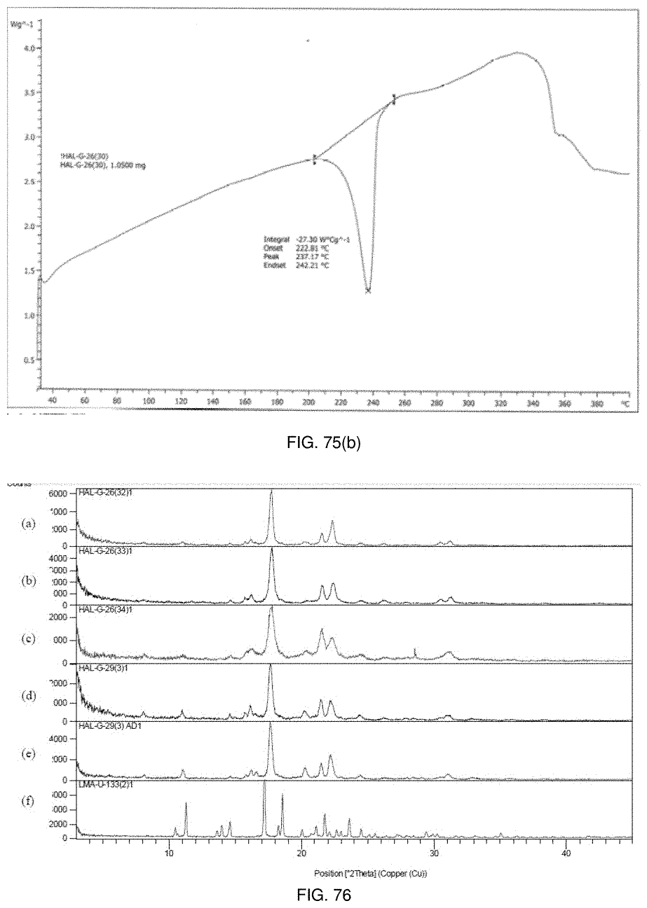

Lots HAL-G-26(30) isolated from THE at a 70 mg scale, afforded a unique XRPD pattern in comparison to the diffraction pattern of the formula (I) free form and the Form A citrate salt (FIG. 71). Observed differences in the XRPD pattern compared to Form A included missing reflections at 4.7, 16 and 17 degrees 2-theta. The crystallinity of the solid was also confirmed by examination under cross polarized light. ¹H-NMR analysis showed an acid: API ratio of 0.5:1 indicating the formation of a hemi-salt. Further inspection of the 1HNMR spectrum showed approximately 1.3 wt % EtOH (FIG. 72). DSC analysis showed a single melting endotherm at 237° C. and no weight loss was observed by TGA up until the onset of melting/decomposition at 192° C. (FIGS. 75(a) and 75(b)). The solubility of Form B in water was estimated to be 190 mg/mL by visual inspection as complete dissolution was observed. Following overnight storage of the solution at ambient conditions, precipitation was observed suggesting hydrate formation. The results obtained from the characterization of formula (I) citrate indicated the presence of a crystalline anhydrate and hydrate hemi-salt form. While the citrate afforded improved aqueous solubility compared to the free form, this counter ion was not selected for further characterization due to the potential for hydrate formation which can often present stability challenges during long term storage. A summary of the results obtained from the characterization of Form B is presented in Table 7.

### (15) Hydrochloric Acid

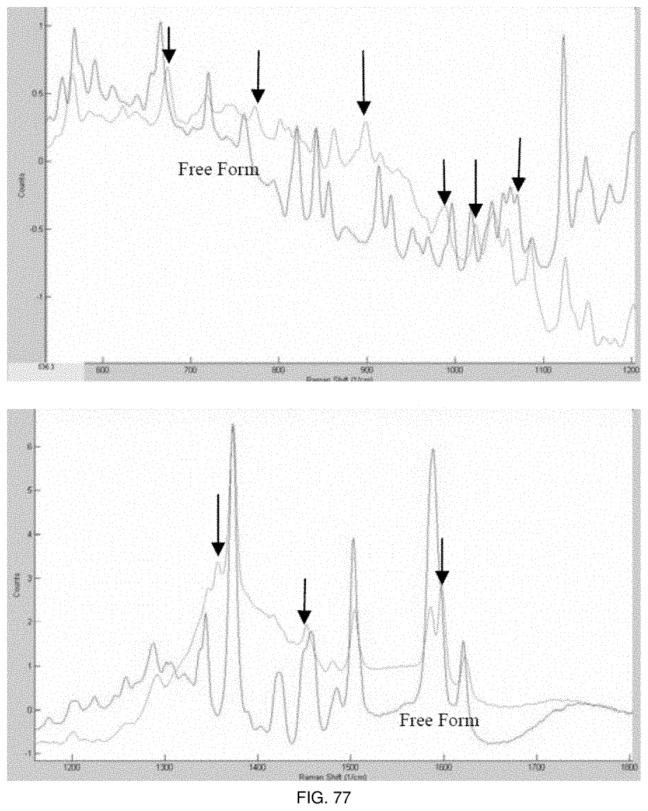

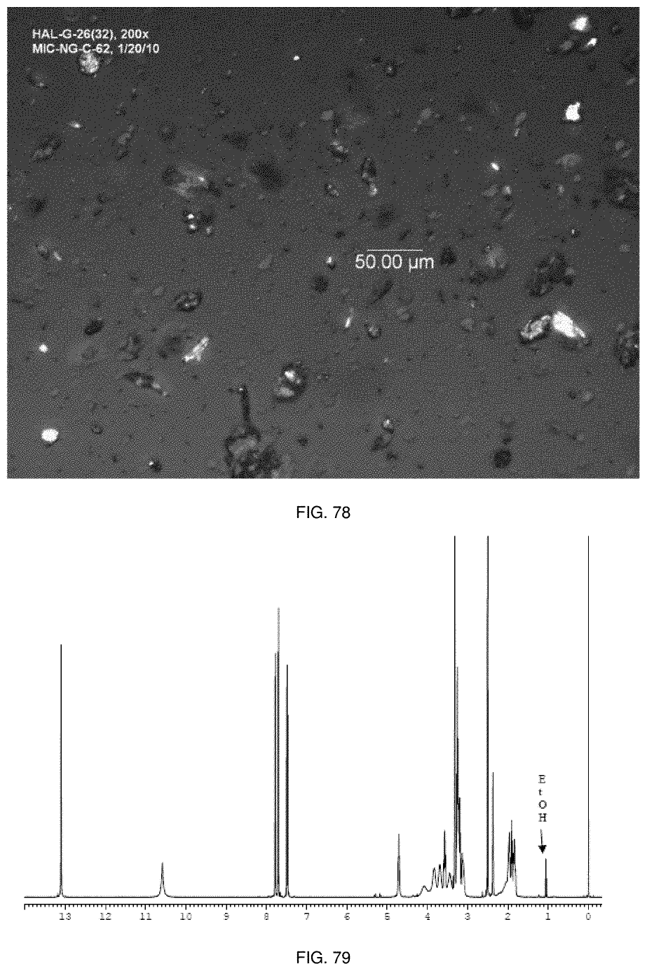

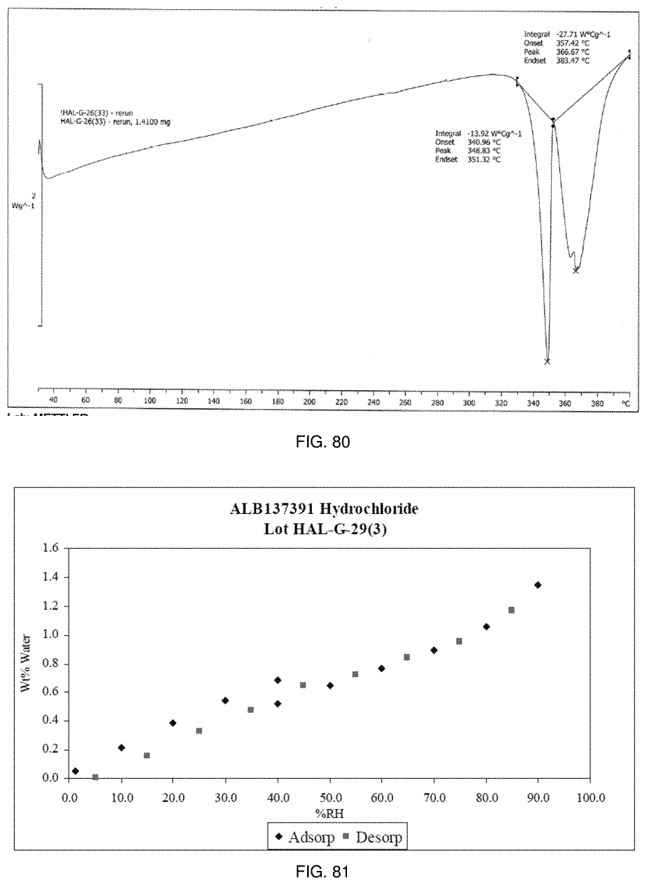

Lots HAL-G-26(32) isolated from EtOH, HAL-G-26(33) from IPA and HAL-G-26(34) from THF at a 70 mg scale, afforded consistent crystalline XRPD patterns which showed differences in comparison to the pattern of the formula (I) free form (FIG. 76). This unique solid form was also obtained from EtOH at a 200 mg scale. A Raman spectrum of the material also exhibited differences in comparison to the spectrum of the free form (FIG. 77). Examination under cross polarized light showed birefringent irregular shaped particles (FIG. 78). The ¹H-NMR spectrum of lot HAL-G-26(32) was consistent with the chemical structure of formula (I) and showed 0.2 wt % EtOH (FIG. 79). Mono-salt formation was confirmed by ICP/OES. Thermal analysis of lot HAL-G-26(33) by DSC showed two endothermic events at 349° C. and 367° C. attributed to melting of the crystalline salt and decomposition (FIG. 80). No weight loss was observed by TGA. The solubility of Form A in water was estimated to be 209 mg/mL by visual inspection as complete dissolution was observed. Given the improved thermal stability and aqueous solubility achieved with this salt in comparison to the free form, the hydrochloride was selected for full characterization.

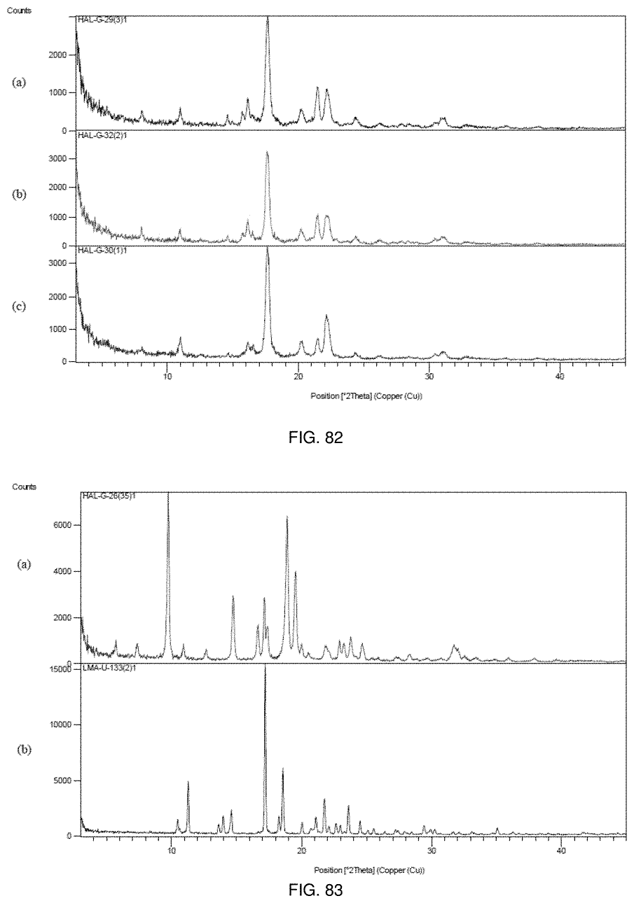

Form A was observed to be slightly hygroscopic adsorbing up to 0.8 wt % water at 60% RH and 1.3 wt % water at 90% RH (FIG. 81). XRPD analysis of the dried material following the program afforded a crystalline pattern that was consistent with the starting material, suggesting no form conversion had occurred during the experiment (FIG. 76). Form A was observed to be stable showing no signs of conversion following seven days of room temperature equilibration in IPA and storage at 60° C. (FIG. 82). These findings suggest that Form A is the thermodynamically favored crystalline form of formula (I) mono-hydrochloride salt. A summary of the results obtained from the characterization of Form A is presented in Table 7.

### (16) Ethane Sulfonic Acid

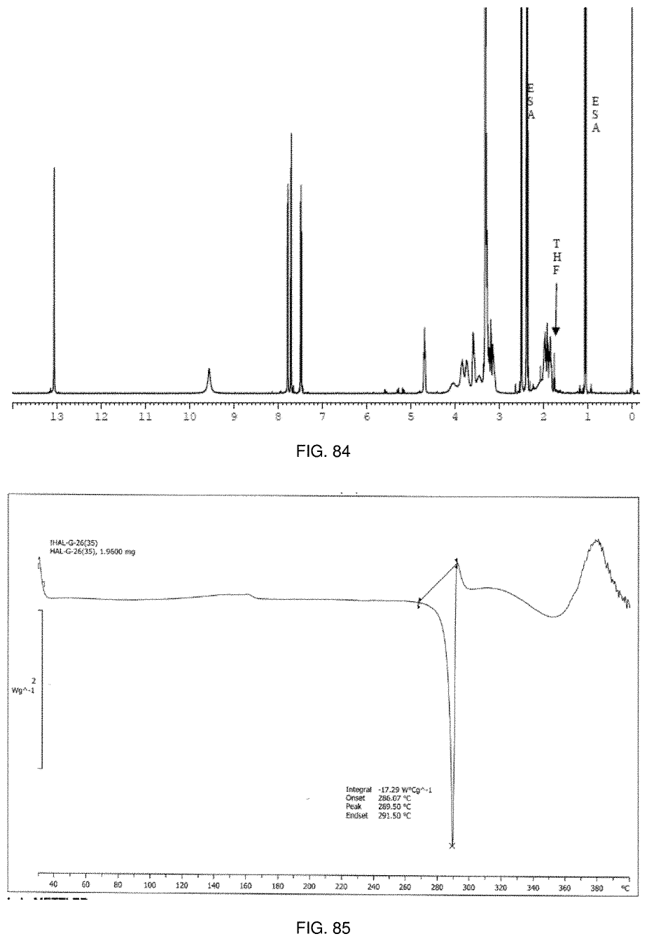

Lots HAL-G-26(35) isolated from THF at a 70 mg scale, afforded a crystalline XRPD pattern which was unique as compared to formula (I) free form and all other synthesized salts (FIG. 83). Examination of the solid under cross polarized light revealed birefringent particles confirming crystallinity. ¹H-NMR analysis of lot HAL-G-26(35) confirmed the chemical structure of formula (I) and showed an acid: API ratio of 1.0:1 indicating the formation of a mono-esylate salt of formula (I) which was designated Form A (FIG. 84). TGA analysis showed no weight loss and a single melting transition was observed by DSC at 290° C. (FIG. 85). The solubility of the esylate salt in water was estimated to Ethanesulfonic acid Lots HAL-G-26(35) isolated from THF at a 70 mg scale, afforded a crystalline XRPD pattern which was unique as compared to formula (I) free form and all other synthesized salts (FIG. 83). Examination of the solid under cross polarized light revealed birefringent particles confirming crystallinity. ¹H-NMR analysis of lot HAL-G-26(35) confirmed the chemical structure of formula (I) and showed an acid:API ratio of 1.0:1 indicating the formation of a mono-esylate salt of formula (I) which was designated Form A (FIG. 84). TGA analysis showed no weight loss and a single melting transition was observed by DSC at 290° C. (FIG. 85). The solubility of the esylate salt in water was estimated to be 380 mg/mL by visual inspection as complete dissolution was observed. While Form A showed desirable physical characteristics, it was not selected for full characterization due to additional testing requirements which would be required for genotoxic impurities if isolated from an alcoholic solvent. A summary of the results obtained from the characterization of Form A is presented in Table 7.

### (17) 1-Hydroxy-2-Napthoic Acid

### Form A

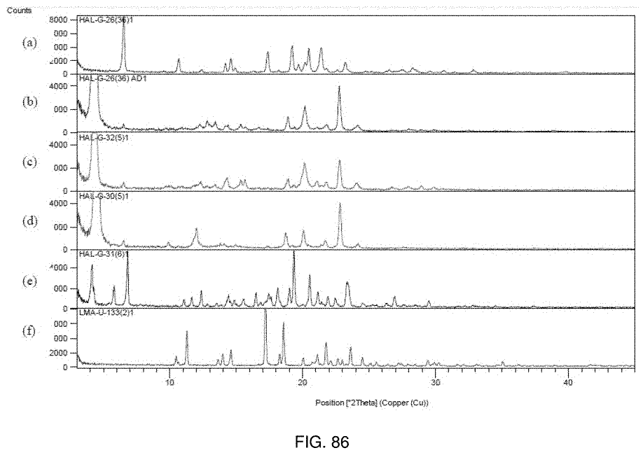

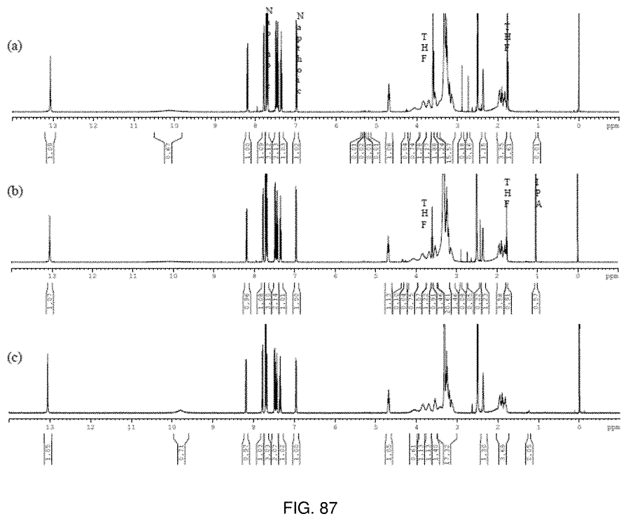

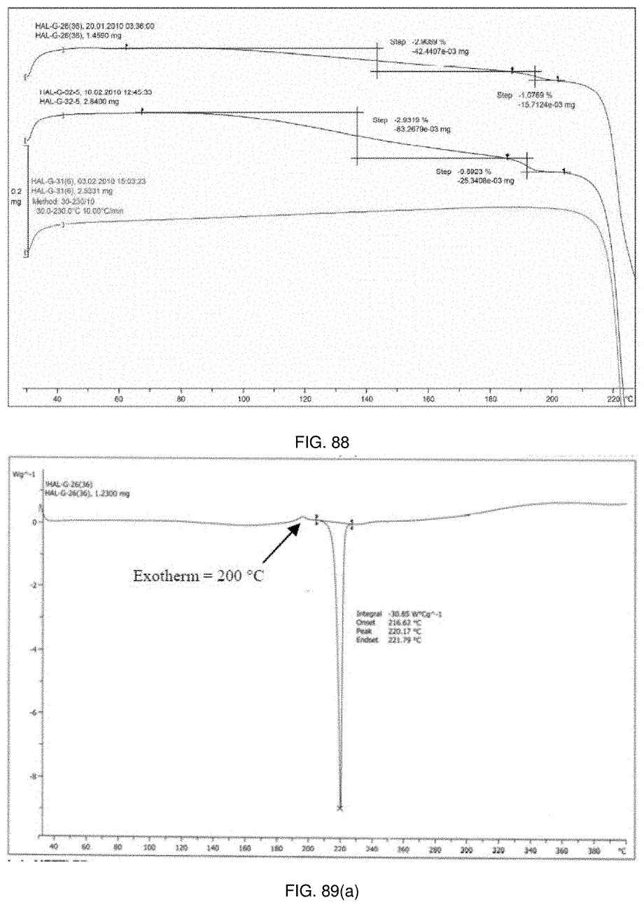

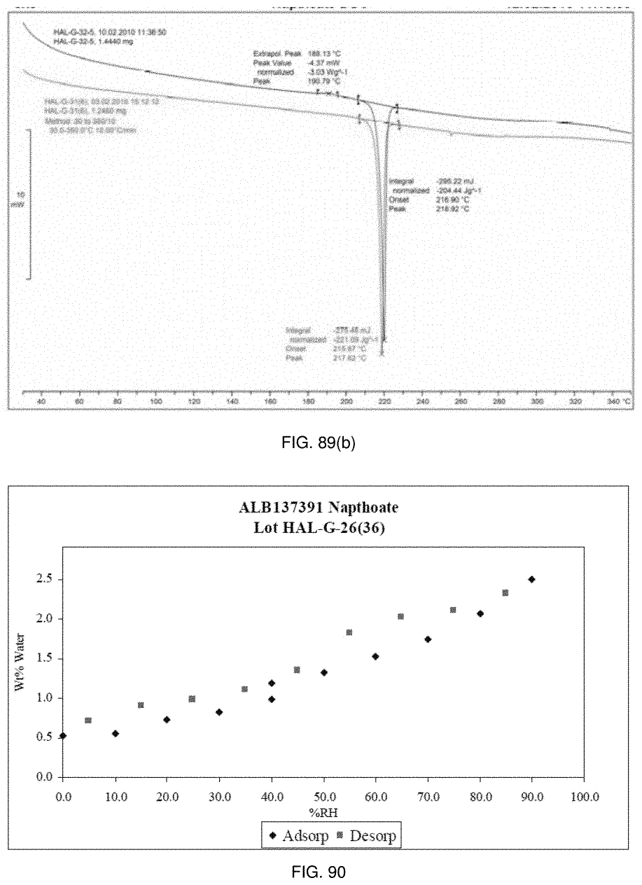

Lots HAL-G-26(36) isolated from THE at a 70 mg scale, afforded a unique crystalline XRPD pattern which showed differences in comparison to the diffraction pattern of formula (I) free form (FIG. 86). The crystallinity of this unique solid was confirmed by examination under cross polarized light. ¹H-NMR analysis of lot HAL-G-26(36) showed an acid: API ratio of 1.1:1 indicating the formation of a mono-naphthoate salt of formula (I) and 5.6 wt % residual THF (FIG. 87). TGA analysis showed a broad weight loss transition from 80-190° C. (2.9 wt %) attributed to loss of residual THF followed by a step transition equivalent to 1.1 wt % loss (FIG. 88). Further analysis by DSC showed a minor exotherm at the same temperature (200° C.) as the TGA step transition followed by melting at 220° C. (FIGS. 89(a) and 89(b)). Further investigation would be required to elucidate the exothermic transition at 200° C. The solubility of the naphthoate salt was initially estimated to be 4.7 mg/mL using a TGA gravimetric method. Solids obtained from the experiment were not analyzed by XRPD however based on additional studies as described below, it can be concluded that the solubility result is indicative of an additional crystalline form (Form C). The naphthoate salt was selected for full characterization in an attempt to include a more stable option over the free form with comparable aqueous solubility.

Form A was observed to be moderately hygroscopic adsorbing up to 1.5 wt % water at 60% RH and 2.5 wt % water at 90% RH (FIG. 90). During the desorption program, the curve stabilized at approximately 2.0 wt % water from 75%-55% RH suggesting formation of a hemi-hydrate (theoretical water content=1.8 wt %). As the relative humidity was reduced further to 45% RH a long equilibration period was noted suggesting dehydration had occurred. The dried solid obtained following the experiment showed a unique XRPD pattern in comparison to the diffraction pattern of Form A (FIG. 86). Subsequent ¹H-NMR analysis confirmed the formation of a unique mono-naphthoate crystalline form of formula (I) which was designated as Form B. This unique form was also observed after seven days of room temperature equilibration in IPA and storage at 60° C. (FIG. 86). Following an overnight room temperature slurry of Form A in water, a unique solid form was observed by XRPD. This material was confirmed to be a crystalline mono-naphthoate salt by ¹H-NMR which was designated Form C (FIG. 86). Further characterization of this crystalline form is described later in this section of Form C.

### Form B

Form B was obtained from Form A after prolonged storage at 60° C., post moisture sorption analysis, and seven days of room temperature equilibration in IPA. Representative XRPD patterns of Form B are presented in FIG. 86. ¹H-NMR analysis of lot HAL-G-32(5) showed an acid: API ratio of 1.0:1 confirming the formation of a mono-naphthoate salt of formula (I). Further inspection of the proton spectrum showed 3.3 wt % residual THF and 1.2 wt % IPA (FIG. 87). TGA analysis afforded a similar thermogram to Form A exhibiting a broad weight loss transition from 80-190° C. (2.9 wt %) attributed to loss of residual THF and/or IPA followed by a step transition equivalent to 0.9 wt % loss (FIG. 88). Further analysis by DSC showed the same transitions observed for Form A including a minor exotherm at 191° C. followed by a melting endotherm at 219° C. (FIGS. 89(a), 89(b)). Further investigation would be required to determine if the DSC exotherm and corresponding TGA step transition are attributed to a solid state conversion or desolvation. A summary of the results obtained from the characterization of Form B is presented in Table 7.

### Form C

Form C was obtained following an overnight room temperature slurry of Form A in water (FIG. 86). ¹H-NMR analysis of lot HAL-G-31(6) showed an acid: API ratio of 1.0:1 confirming the formation of a mono-naphthoate salt of formula (I). Further inspection of the ¹H-NMR spectrum revealed no residual solvent and no weight loss was observed by TGA (FIG. 87). DSC analysis showed a single endotherm at 218° C. attributed to melting of Form C (FIGS. 89(a) and 89(b)). The solubility of Form C in water was determined to be 2.3 mg/mL by HPLC. Further investigation would be required to better understand the stability relationships between Forms A, B and C of formula (I) mono-naphthoate salt.

### (18) Succinic Acid

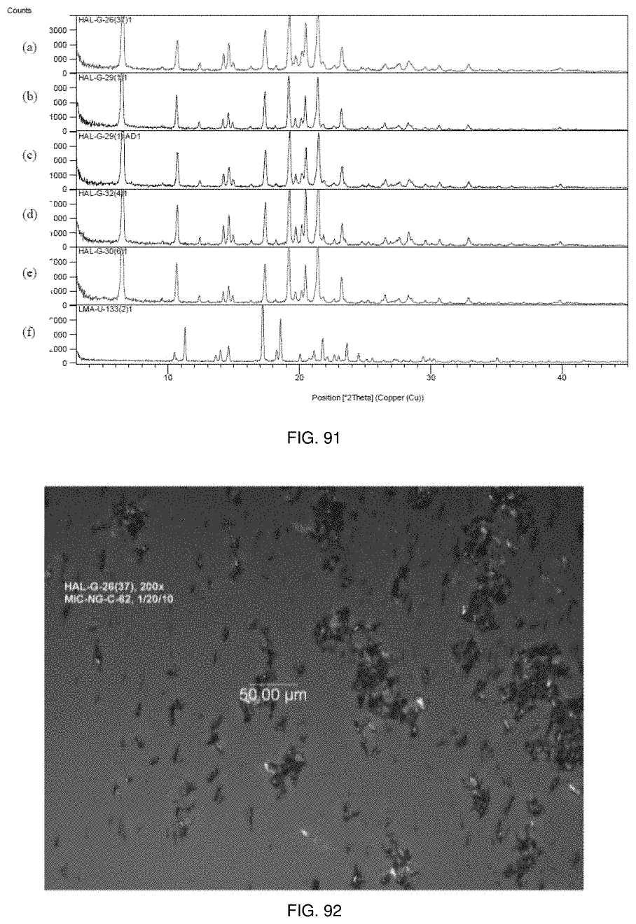

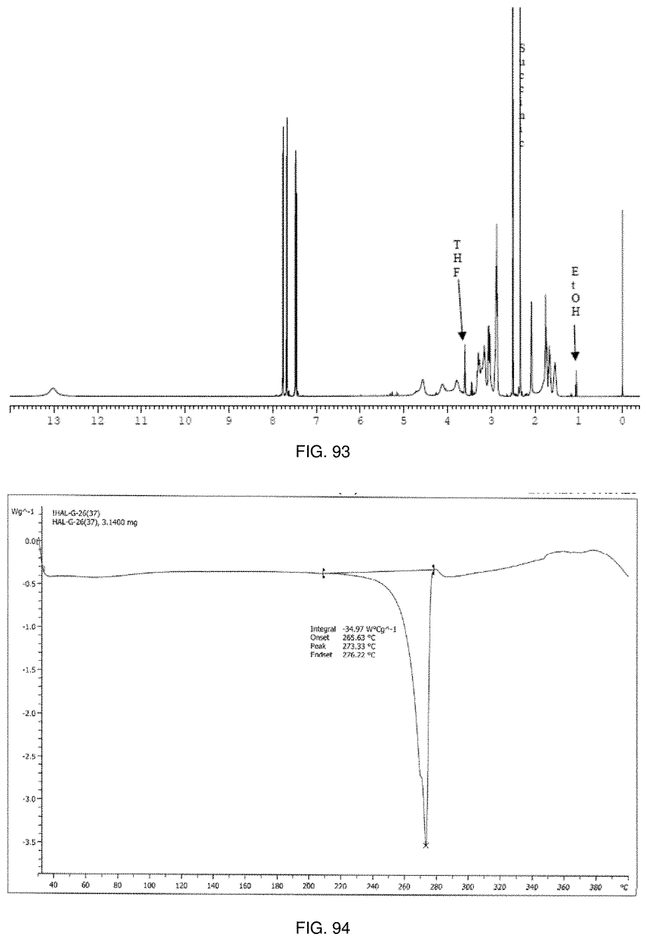

Lots HAL-G-26(37) isolated from THF at a 70 mg scale and lot HAL-G-29(1) at a 200 mg scale, afforded consistent crystalline XRPD patterns which showed differences in comparison to the pattern of the formula (I) free form (FIG. 91). Examination of lot HAL-G-26(37) under cross polarized light revealed birefringent particles with an acicular morphology (FIG. 92). ¹H-NMR analysis of lot HAL-G-26(37) confirmed the chemical structure of formula (I)) and showed an acid: API ratio of 1.1:1 indicating the formation of a mono-succinate salt of formula (I) which was designated Form A (FIG. 93). Thermal analysis of the succinate salt by DSC showed a single endothermic event at 273° C. which was attributed to melting of the crystalline salt (FIG. 94). No weight loss was observed by TGA. The solubility of Form A in water was estimated to be 140 mg/mL by visual inspection as complete dissolution was observed. This salt was selected for full characterization based upon thermal stability, high degree of crystallinity, and improved aqueous solubility observed in comparison to the free form.

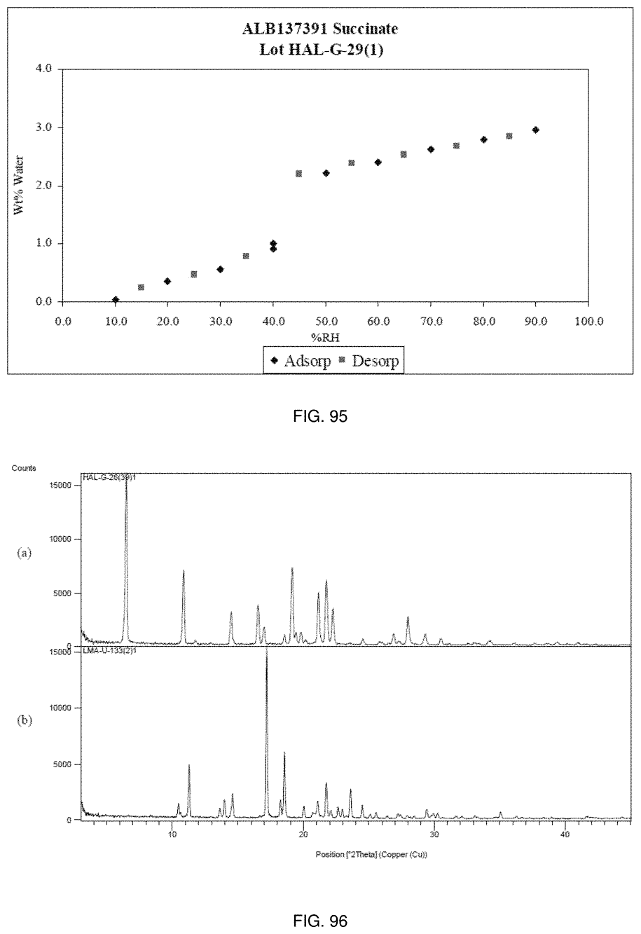

The Form A succinate salt showed a sharp increase in water sorption from 40%-50% RH by gravimetric moisture sorption (FIG. 95). The salt adsorbed approximately 2 wt % water which is equivalent to a hemi-hydrate of formula (I) mono-succinate. During the desorption program a sharp decrease in water sorption was observed from 45-35% RH indicative of dehydration. These findings indicate that the hydrate form is unstable below 45% RH. XRPD analysis of the dried material following the program afforded a crystalline pattern that was consistent with the starting material providing further evidence that dehydration had occurred during the experiment (FIG. 91). Form A was observed to be stable showing no signs of conversion following seven days of room temperature equilibration in IPA and storage at 60° C. (FIG. 91). These findings suggest that Form A is the thermodynamically favored crystalline form of formula (I) mono-succinate salt. A summary of the results obtained from the characterization of Form A is presented in Table 7.

### (19) Glutaric Acid

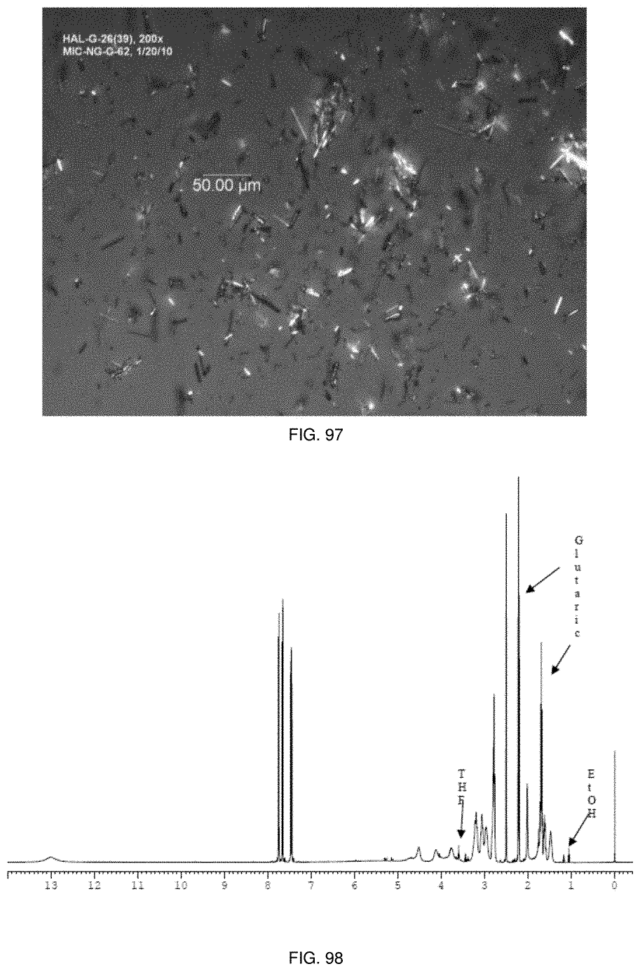

Lots HAL-G-26(39) isolated from THF at a 70 mg scale afforded a crystalline XRPD pattern which was unique as compared to formula (I) free form and all other synthesized salts (FIG. 96). This solid form was also observed to be crystalline exhibiting birefringent acicular particles under cross polarized light (FIG. 97). ¹H-NMR analysis of lot HALG-26(39) confirmed the chemical structure of formula (I) and showed an acid: API ratio of 1.0:1 indicating the formation of a mono-glutarate salt of formula (I) which was designated Form A. Further inspection of the ¹H-NMR spectrum showed approximately 0.9 wt % THF and 0.1 wt % EtOH (FIG. 98). Thermal analysis of Form A by DSC showed a single endothermic event at 269° C. which was attributed to melting of the crystalline salt. No weight loss was observed by TGA. The solubility of the Form A mono-salt in water was estimated to be in excess of 400 mg/mL by visual inspection as complete dissolution was observed. While this salt showed desirable physical characteristics, it was not selected for further characterization due to the counter ion's lack of prevalence in pharmaceutical drug products. A summary of the results obtained from the characterization of Form A is presented in Table 7.

### (20) L-Pyroglutamic Acid

### Form A

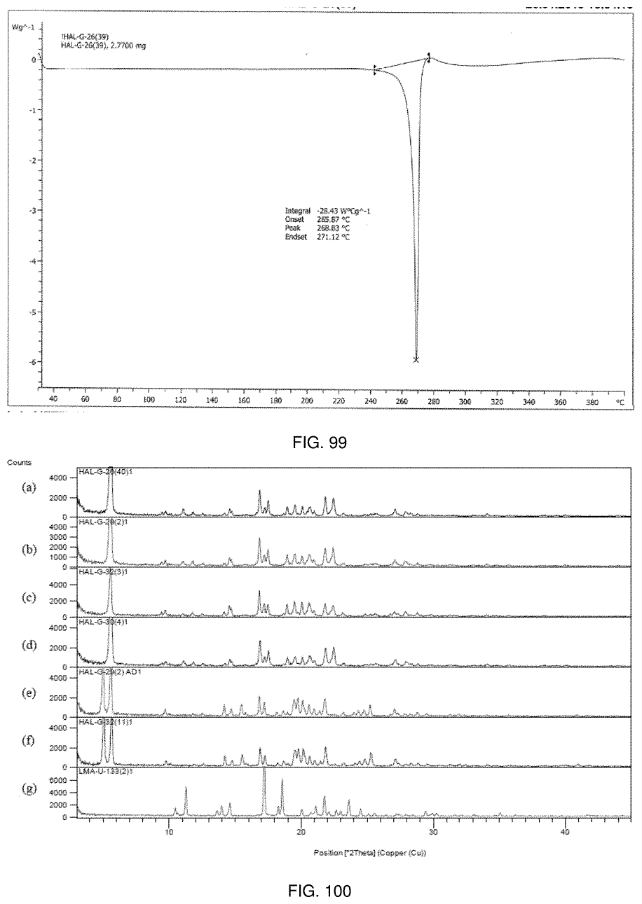

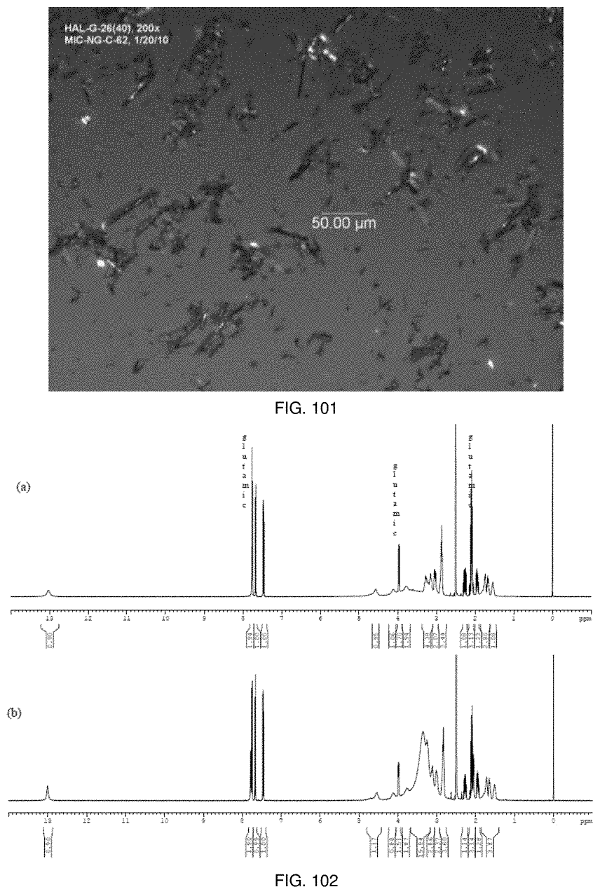

Lots HAL-G-26(40) isolated from THF at a 70 mg scale, afforded a crystalline XRPD pattern which was unique as compared to formula (I) free form and all other synthesized salts (FIG. 100). This solid form was also obtained at a 200 mg scale from the same solvent. Analysis by optical microscopy confirmed the solid was crystalline exhibiting birefringent acicular and blade like particles under cross polarized light (FIG. 101). ¹H-NMR analysis of lot HAL-G-26(40) confirmed the chemical structure of formula (I) and showed an acid to API ratio of 1.1:1 indicating the formation of a mono-glutamate salt of formula (I) which was designated Form A (FIG. 102). Thermal analysis of Form A by DSC showed a single endothermic event at 241° C. which was attributed to melting of the crystalline salt (FIG. 99). No weight loss was observed by TGA. The solubility of the Form A mono-salt in water was estimated to be in excess of 400 mg/mL by visual inspection as complete dissolution was observed. This salt was selected for full characterization based upon the high degree of crystallinity observed by microscopy and XRPD, and improved aqueous solubility in comparison to the free form.

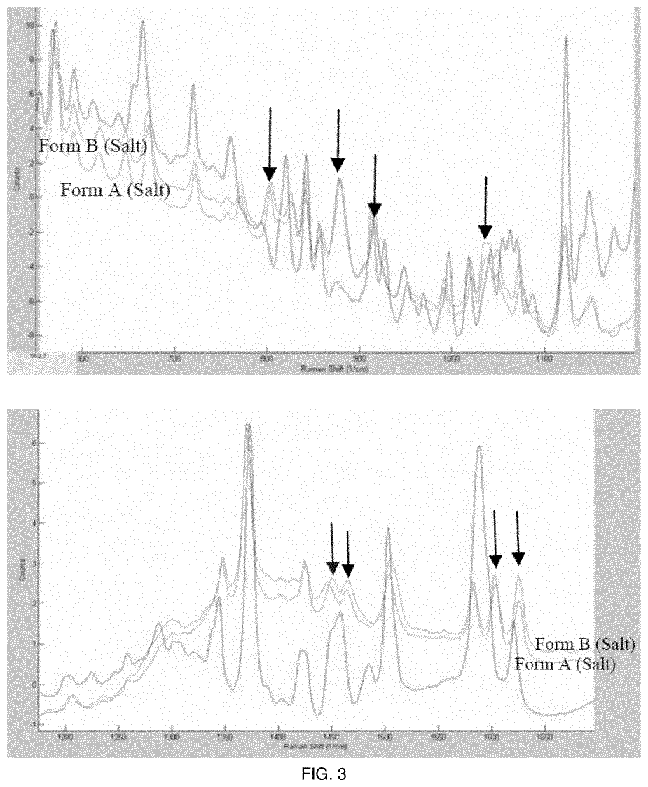

Form A was observed to be stable showing no signs of conversion following seven days of room temperature equilibration in IPA and storage at 60° C. (FIG. 100). These findings suggest that Form A is the thermodynamically favored crystalline form of formula (I) mono-glutamate salt. The Form A glutamate salt was observed to be relatively non-hygroscopic until exposure to relative humidity conditions greater than 80% RH (FIG. 2). A sharp increase in water sorption was observed from 80% RH to 90% RH resulting in deliquescence of the salt. Upon desorption, the salt likely formed a monohydrate (theoretical=4.0 wt %) which was observed to be stable down to 0% RH. The dried solid obtained following the experiment afforded a crystalline XRPD pattern which showed differences in comparison to the diffraction pattern of Form A (FIG. 100). A Raman spectrum of this material also exhibited differences in comparison to the spectrum of the Form A salt (FIG. 3). Subsequent ¹H-NMR analysis confirmed the formation of a unique mono-glutamate crystalline form of formula (I) which was designated as Form B (FIG. 102).

### Form B

As presented above, Form B was obtained following moisture sorption analysis of Form A and subsequent drying. In an effort to generate additional Form B for further characterization, a brief humidity study was performed as described below: approximately 30 mg of Form A mono-glutamate salt [lot HAL-G-29(2)] was stored uncovered in a small glass container in a desiccator containing an aqueous saturated solution of Na₂HPO₄.12H₂O to achieve a relative humidity of 95%.

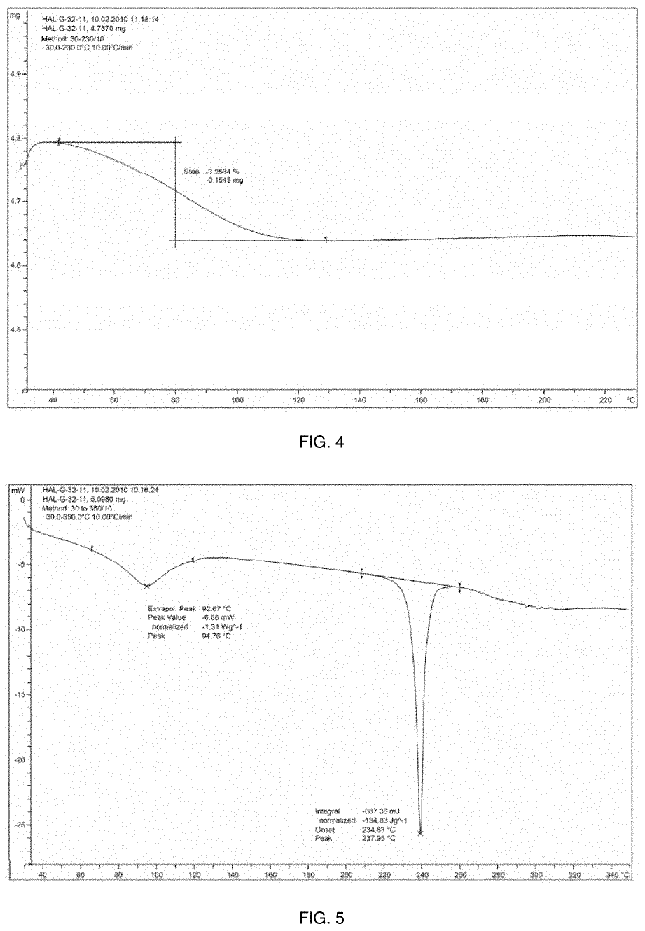

After five hours of equilibration at 95% RH, the solid had deliquesced. Following overnight exposure of the sample to the lab environment (˜20% RH), a dry solid was obtained which had adhered to the sample container. XRPD analysis of the solid afforded a crystalline XRPD pattern similar to that of Form B (FIG. 100). TGA analysis of Form B showed 3.3 wt % loss from 40-120° C. which is comparable to the theoretical water content (4.0 wt %) of a monohydrate mono-glutamate salt (FIG. 4). The DSC thermogram of Form B showed two endothermic transitions at 95° C. and 238° C. attributed to dehydration and melting respectively (FIG. 5). The results obtained from the characterization of formula (I) glutamate indicated the presence of a crystalline anhydrate (Form A) and potential mono-hydrate (Form B). A summary of the results obtained from the characterization of Form B is presented in Table 7.

The above results indicate that the present disclosure has prepared various formula (I) salts of different crystal forms, compared with formula (I) methanesulfonate, phosphate, hydrochloride, succinate, 1-hydroxy-2-naphthoate and L-pyroglutamate have better properties with better crystallinity, solubility or stability. In each crystal forms, Form A has more excellent properties. Among the above various salts, the hydrochloride has the best overall performance due to its higher water solubility, better stability, and less hygroscopicity.

In summary, the present disclosure provides various salts of formula (I), as well as crystal forms and methods for their preparation. The inventors of the present invention conducted various researches under such a situation as mentioned above. As a result, they found that a hydrochloride salt of Formula (I) has at least one or more of such characteristics as (1) it has superior stability, (2) it shows superior crystallinity, (3) it shows high water solubility, (4) it does not show deliquescent property, (5) it shows superior flowability, (6) it shows superior tableting property, (7) it can be manufactured with less environmental load, (8) it shows less hygroscopicity, and (9) it can be manufactured in a large scale, and therefore it is useful as a bulk drug for medicaments than free base shown in formula (I), and thus they accomplished the present invention.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

TABLE 7

| | SM | | Solvent | | CI | | | | | |
|---|---|---|---|---|---|---|---|---|---|---|
| | Amt | | Amt | | Amt | Temp | | | Yield | XRPD |
| NB Code | (mg) | Solvent | (mL) | 1.05M eq. CI | (mL) | (° C.) | Precip | Isolation | (mg) | (Pattern) |
| HAL-G-26-1 | 70.3 | EtOH | 1.0 | Adipic | 0.50 | 60 | Y | Filter | 67.9 | A |
| HAL-G-26-2 | 70.8 | IPA | 2.0 | Adipic | 0.50 | 60 | Y | Filter | 77.3 | A |
| HAL-G-26-3 | 70.7 | THF | 6.0 | BSA | 0.50 | 60 | Y | Filter | 97.8 | A |
| HAL-G-26-4 | 69.9 | IPA | 2.0 | BSA | 0.50 | 60 | Y | Filter | 94.5 | B |
| HAL-G-26-5 | 70.0 | THF | 6.0 | HBr | 0.50 | 60 | Y | Filter | 30.2 | A* |
| HAL-G-26-6 | 69.9 | IPA | 2.0 | HBr | 0.50 | 60 | Y | Filter | 77.0 | A* |
| HAL-G-26-7 | 69.9 | THF | 6.0 | L-Mandelic | 0.50 | 60 | N | Evap | NA | A* |
| HAL-G-26-8 | 70.0 | IPA | 2.0 | L-Mandelic | 0.50 | 60 | Y^{D} | Filter | 6.3 | A* |
| HAL-G-26-9 | 70.6 | THF | 6.0 | Fumaric | 1.00 | 60 | Y | Filter | 78.5 | A |
| HAL-G-26-10 | 70.8 | IPA | 2.0 | Fumaric | 1.00 | 60 | Y | Filter | 75.5 | A |
| HAL-G-26-11 | 70.1 | THF | 6.0 | Benzoic | 0.50 | 60 | Y | Filter | 73.1 | A |
| HAL-G-26-12 | 70.7 | IPA | 2.0 | Benzoic | 0.50 | 60 | Y | Filter | 74.0 | A |
| HAL-G-26-13 | 69.8 | THF | 6.0 | MSA | 0.49 | 60 | Y | Filter | 88.7 | A |
| HAL-G-26-14 | 70.4 | IPA | 2.0 | MSA | 0.50 | 60 | Y | Filter | 68.1 | A |
| HAL-G-26-15 | 70.7 | THF | 6.0 | L-Malic | 0.50 | 60 | Y | Filter | 71.3 | A |
| HAL-G-26-16 | 70.3 | IPA | 2.0 | L-Malic | 0.50 | 60 | Y | Filter | 73.6 | A |
| HAL-G-26-17 | 70.1 | THF | 6.0 | D-glucoronic | 0.50 | 60 | Y | Filter | 109.9 | A |
| HAL-G-26-18 | 70.2 | IPA | 2.0 | D-glucoronic | 0.50 | 60 | Y | Filter | 93.0 | A |
| HAL-G-26-19 | 70.5 | THF | 6.0 | Sorbic | 0.50 | 60 | Y^{D} | Filter | Deliq | — |
| HAL-G-26-20 | 70.2 | IPA | 2.0 | Sorbic | 0.50 | 60 | Y^{D} | Filter | 54.0 | A |
| HAL-G-26-21 | 69.8 | THF | 6.0 | Phosphoric | 0 49 | 60 | Y | Filter | 78.2 | A |
| HAL-G-26-22 | 70.2 | IPA | 2.0 | Phosphoric | 0.50 | 60 | Y | Filter | 75.2 | A |
| HAL-G-26-23 | 70.0 | THF | 6.0 | Sulfuric | 0.50 | 60 | Y | Filter | 79.6 | A |
| HAL-G-26-24 | 70.4 | IPA | 2.0 | Sulfuric | 0.50 | 60 | Y | Filter | 77.7 | B |
| HAL-G-26-25 | 70.0 | THF | 6.0 | L-tartaric | 0.50 | 60 | Y | Filter | 68.5 | A |
| HAL-G-26-26 | 70.0 | IPA | 2.0 | L-tartaric | 0.50 | 60 | Y | Filter | 77.7 | B |
| HAL-G-26-27 | 70.1 | THF | 6.0 | p-TSA | 0.50 | 60 | Y | Filter | 78.1 | A* |
| HAL-G-26-28 | 70.5 | IPA | 2.0 | p-TSA | 0.50 | 60 | Y | Filter | 95.1 | A |
| HAL-G-26-29 | 69.8 | MeOH | 0.5 | Citric | 0.49 | 60 | Y | Filter | 59.2 | A |
| HAL-G-26-30 | 69.9 | THF | 6.0 | Citric | 0.50 | 60 | Y | Filter | 84.2 | B |
| HAL-G-26-31 | 70.2 | IPA | 2.0 | Citric | 0.50 | 60 | Y | Filter | 74.2 | A |
| HAL-G-26-32 | 70.2 | EtOH | 1.0 | HCl | 0.50 | 60 | Y | Filter | 55.9 | A |
| HAL-G-26-33 | 70.1 | IPA | 2.0 | HCl | 0.50 | 60 | Y | Filter | 52.1 | A |
| HAL-G-26-34 | 70.8 | THF | 6.0 | HCl | 0.50 | 60 | Y | Filter | 48.8 | A |
| HAL-G-26-35 | 70.0 | THF | 6.0 | Ethanesulfonic | 0.50 | 60 | Y | Filter | 85.0 | A |
| HAL-G-26-36 | 70.6 | THF | 6.0 | 1-hydroxy- | 0.50 | 60 | Y | Filter | 101.8 | A |
| | | | | 2-napthoic | | | | | | |
| HAL-G-26-37 | 70.3 | THF | 6.0 | Succinic | 0.50 | 60 | Y | Filter | 75.3 | A |
| HAL-G-26-38 | 70.5 | THF | 6.0 | Acetic | 0.50 | 60 | N | Evap | NA | A |
| HAL-G-26-39 | 70.8 | THF | 6.0 | Glutaric | 0.50 | 60 | Y | Filter | 59.9 | A |
| HAL-G-26-40 | 70.1 | THF | 6.0 | 2-pyrrolidine- | 0.50 | 60 | Y | Filter | 62.2 | A |
| | | | | 5-carboxylic | | | | | | |
| HAL-G-26-41 | 70.2 | THF | 6.0 | L-proline | 0.50 | 60 | Y | Filter | 20.8 | A |

*Semicrystalline XRPD pattern
^{D}precipitation upon cooling all other ppt upon [symbol] addition [symbol]
additional [symbol] at 6.5 degrees 2-theta [symbol]
indicates data missing or illegible when filed

1. A salt of the molecule with the following formula (I)

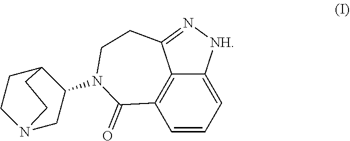 [SMILES: O=C1c2cccc3[nH]nc(c23)CCN1[C@@H]1CN2CCC1CC2]

2. A salt according to claim 1, wherein the salt is one or more of the adipate, benzenesulphonate, hydrobromide, fumarate, benzoate, methanesulfonate, L-malate, d-glyconate, sorbate, phosphate, sulfate, L-tartrate, p-methylbenzenesulphonate, citrate, hydrochloride, ethanesulfonate, 1-hydroxy-2-naphthoate, succinate, acetate, glutarate, or L-pyroglutamate salt.

3. A salt according to claim 2, wherein the salt is one or more of the methanesulfonate, phosphate, hydrochloride, succinate, 1-hydroxy-2-naphthoate, or L-pyroglutamate salt.

4. A salt according to claim 3, which comprises the methanesulfonate salt.

5. A salt according to claim 4 characterized substantially by at least one of the following powder x-ray diffraction pattern peaks expressed in terms of diffraction angle 2θ (±0.2°) as measured with copper Kα radiation chosen from: 10.2°, 12.9°, and 23.9°.

6. A salt according to claim 3, which comprises the phosphate salt.

7. A salt according to claim 6 characterized substantially by at least one of the following powder x-ray diffraction pattern peaks expressed in terms of diffraction angle 2θ (±0.2°) as measured with copper Kα radiation chosen from: 9.7°, 12.3°, 20.1°, and 21.3°.

8. A salt according to claim 3, which comprises the hydrochloride salt.

9. A salt according to claim 8 characterized substantially by at least one of the following powder x-ray diffraction pattern peaks expressed in terms of diffraction angle 2θ (±0.2°) as measured with copper Kα radiation chosen from: 17.7°, 21.5°, and 22.3°.

10. A salt according to claim 3, which comprises the succinate salt.

11. A salt according to claim 10 characterized substantially by at least one of the following powder x-ray diffraction pattern peaks expressed in terms of diffraction angle 2θ (±0.2°) as measured with copper Kα radiation chosen from: 4.3°, 20.1°, and 22.7°.

12. A salt according to claim 3, which comprises the 1-hydroxy-2-naphthoate salt.

13. A salt according to claim 12 characterized substantially by at least one of the following powder x-ray diffraction pattern peaks expressed in terms of diffraction angle 2θ (±0.2°) as measured with copper Kα radiation chosen from: 6.5°, 10.7°, 19.2°, and 20.5°.

14. A salt according to claim 3, which comprises L-pyroglutamate salt.

15. A salt according to claim 14 characterized substantially by at least one of the following powder x-ray diffraction pattern peaks expressed in terms of diffraction angle (2θ) (±0.2°) as measured with copper Kα radiation chosen from: 5.5°, 16.8°, and 22.4°.

16. A polymorph of a compound having the structure represented by formula (I)

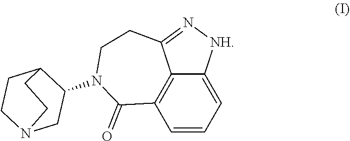 [SMILES: O=C1c2cccc3[nH]nc(c23)CCN1[C@@H]1CN2CCC1CC2]

17. The polymorph of claim 16, which is characterized substantially by at least one of the following powder x-ray diffraction pattern peak expressed in terms of diffraction angles (2θ) (±0.2°) as measured with copper Kα radiation chosen from: 11.3°, 14.6°, 21.8° and 23.6°.

18. A pharmaceutical formulation comprising a salt or a polymorph of a compound having a structure represented by formula (I), as defined in claim 16, and a pharmaceutically acceptable excipient.

19. A method of treating a disease, comprising administering to a subject in need of such treatment a therapeutically effective amount of a salt or a polymorph of a compound having a structure represented by formula (I), as defined in claim 16.

20. The method according to claim 19, wherein the disease comprises inflammatory bowel disease (including but not limited to ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, drug/toxin-induced cognitive impairment (e.g., from alcohol, barbiturates, vitamin deficiencies, recreational drugs, lead, arsenic, mercury), disease-induced cognitive impairment (e.g., arising from Alzheimer's disease (senile dementia), vascular dementia, Parkinson's disease, multiple sclerosis, AIDS, encephalitis, trauma, renal and hepatic encephalopathy, hypothyroidism, Pick's disease, Korsakoffs syndrome and frontal and subcortical dementia), hypertension, bulimia, anorexia, obesity, cardiac arrhythmias, gastric acid hypersecretion, ulcers, pheochromocytoma, progressive supramuscular palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, attention deficit hyperactivity disorder (ADHD) and Tourette's Syndrome.
